(19) United States
(12) Patent Application Publication
(10) Pub. No.: US 2025/0313622 A1
(43) Pub. Date: Oct. 9, 2025

(54) CHIMERIC ANTIGEN RECEPTOR (CAR) CONSTRUCTS WITH NK RECEPTOR SIGNALING DOMAIN

(71) Applicant: H. LEE MOFFITT CANCER CENTER AND RESEARCH INSTITUTE INC., Tampa, FL (US)

(72) Inventors: Jose Conejo-Garcia, Durham, NC (US); Jose Alejandro Guevara, Tampa, FL (US)

(21) Appl. No.: 18/860,458

(22) PCT Filed: May 2, 2023
(86) PCT No.: PCT/US2023/066498

§ 371 Date: Oct. 25, 2024

Related U.S. Application Data
(60) Provisional application No. 63/364,085, filed on May 3, 2022.
(60) Provisional application No. 63/364,760, filed on May 16, 2022.
(60) Provisional application No. 63/365,291, filed on May 25, 2022.

(51) Int. Cl. C07K 16/28; A61K 40/11; A61K 40/31; A61K 40/42; A61P 35/00; C07K 14/705; C12N 5/0783

(52) U.S. Cl. CPC C07K 16/28; A61K 40/11; A61K 40/31; A61K 40/4202; A61P 35/00; C07K 14/7056; C12N 5/0636; C07K 2317/622; C07K 2319/03; C12N 2510/00

(57) ABSTRACT

Disclosed herein are chimeric antigen receptor (CAR) polypeptides that can be used with adoptive cell transfer to target and kill cancers. As with other CARs, the disclosed CAR polypeptides contain an ectodomain that contains a binding domain, a hinge domain, a transmembrane (TM) domain, and an endodomain that contains a signaling region. Unlike other CARs, however, the endodomain of the disclosed CAR polypeptides contains an intracellular domain of an NK cell receptor and does not require the signaling domain from CD3 zeta (CD3ζ). Also disclosed are immune effector cells, such as T cells or Natural Killer (NK) cells, that are engineered to express these CARs. Therefore, also disclosed are methods of providing an anti-tumor immunity in a subject with a tumor associated antigen-expressing cancer that involves adoptive transfer of the disclosed immune effector cells engineered to express the disclosed CARs.

This application claims benefit of U.S. Provisional Application No. 63/364,085, filed May 3, 2023, U.S. Provisional Application No. 63/364,760, filed May 16, 2023, and and U.S. Provisional Application No. 63/365,291, filed May 25, 2022, which are hereby incorporated herein by reference in their entireties.

### SEQUENCE LISTING

This application contains a sequence listing filed in ST.26 format entitled “320803_2660_Sequence_Listing” created on May 2, 2023. The content of the sequence listing is incorporated herein in its entirety.

### BACKGROUND

Surgery, radiation therapy, and chemotherapy have been the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed.

A major advance for anti-cancer T cell therapy is the chimeric antigen receptor (CAR), which is a single chain variable fragment (scFv) derived from an antibody fused to the signaling domains of a T cell receptor (TCR) (Davila, M. L., et al., Oncoimmunology, 2012. 1(9):1577-1583). The intracellular domain of a first-generation CAR includes only CD3ζ, while second-generation CARs also include co-stimulatory domains such as CD28 or 41BB. These second-generation CAR domains support highly-efficacious tumor killing in mice and led to the clinical evaluation of CAR T cell therapies in patients. The potential of CD19-targeted CAR T cells was confirmed by reports of complete remission rates of 90% for patients with B cell acute lymphoblastic leukemia (B-ALL) (Davila, M. L., et al., Sci Transl Med, 2014. 6(224):224ra25; Maude, S. L., et al., N Engl J Med, 2014. 371(16):1507-17). However, poor CAR T cell persistence and excessive T cell activation contribute to relapses and severe toxicities, respectively, and suggest a critical need to understand CAR T cell biology (Gangadhar, T. C. and R. H. Vonderheide, Nat Rev Clin Oncol, 2014. 11(2):91-9). Furthermore, relapses and toxicities have been seen with all second-generation CARs suggesting that the addition of co-stimulatory domains to CARs improved efficacy, but at the cost of biologic complications.

### SUMMARY

Disclosed herein are chimeric antigen receptor (CAR) polypeptides that can be used with adoptive cell transfer to target and kill cancers. As with other CARs, the disclosed CAR polypeptides contain an ectodomain that contains a binding domain, a hinge domain, a transmembrane (TM) domain, and an endodomain that contains a signaling region. Unlike other CARs, however, the endodomain of the disclosed CAR polypeptides contains an intracellular domain of an NK cell receptor and does not require the signaling domain from CD3 zeta (CD3ζ).

The disclosed CAR polypeptides therefore utilize a completely different signaling route (NKG2D/DAP10) than current CAR-T cells (CD3, OX40, ICOS, etc.). Because the signaling route does not rely on CD3ζ or ZAP70 signaling, it may be insensitive to PD-1-mediated inhibition. Moreover, studies have shown that in T cells, NKG2D/DAP10 results in target killing, memory formation (Perez C, et al. J Immunother Cancer 2019), can substitute the need for CD4 help help′ (Zloza A, et al. Nat Med 2012 18:422-8), mediate resistance to immune suppression by TGF-β, and enhanced migratory properties.

Therefore, disclosed herein is a CAR polypeptide having a ligand binding domain, a hinge domain, a transmembrane domain, and an endodomain that contains an NK cell receptor intracellular domain, or fragment thereof capable of activating NK cell killing. In some embodiments, the CAR polypeptide further contains the NK cell receptor extracellular domain, or a fragment thereof.

The disclosed polypeptides can in some embodiments further contain in the endodomain additional motifs, such as one or more DAP10 or other signaling motifs, e.g. OX40, ICOS, CD3, etc.. However, in some embodiments, the CAR polypeptides lack any other intracellular signaling or co-stimulatory domains, such as a CD3ζ, 41 BB, and/or CD28 domains.

### NK Receptors

### NKG2D

In some embodiments, the NK cell receptor is hNKG2D. In some embodiments, the NK cell receptor intracellular (IC) domain comprises the amino acid sequence MGWIRGRRSRHSWEMSEFHNYNLDLKKSDFSTRWQKQRCPVVKSKCRENAS (SEQ ID NO:1), or a variant/fragment thereof having at least 70%, 85%, 90%, 95%, 98% or 99% identity to SEQ ID NO:1. In some embodiments, the TM has the amino acid sequence PFFFCCFIAVAMGIRFIIMVTIW (SEQ ID NO:2), or a variant/fragment thereof having at least 70%, 85%, 90%, 95%, 98% or 99% identity to SEQ ID NO:2. Therefore, in some embodiments, the CAR comprises the hNKG2D TM/IC domains having the amino acid sequence PFFFCCFIAVAMGIRFIIMVTIWMGWIRGRRSRHSWEMSEFHNYNLDLKKSDFSTRWQ KQRCPVVKSKCRENAS (SEQ ID NO:3), or a variant/fragment thereof 64, 65, 66, 67, 68, 69, 70, 71, 72, 73, or 74 amino acids in length and having at least 70%, 85%, 90%, 95%, 98% or 99% identity to SEQ ID NO:3 that is capable of integrating into a plasma membrane and binding DAP10 when crosslinked. In some embodiments, the extracellular domain has the amino acid sequence MGWIRGRRSRHSWEMSEFHNYNLDLKKSDFSTRWQKQRCPVVKSKCRENAS (SEQ ID NO:37), or a variant/fragment thereof having at least 70%, 85%, 90%, 95%, 98% or 99% identity to SEQ ID NO:37. In some embodiments, hNKG2D has the amino acid sequence MGWIRGRRSRHSWEMSEFHNYNLDLKKSDFSTRWQKQRCPVVKSKCRENASPFFFC CFIAVAMGIRFIIMVTIWSAVFLNSLFNQEVQIPLTESYCGPCPKNWICYKNNCYQFFDE SKNWYESQASCMSQNASLLKVYSKEDQDLLKLVKSYHWMGLVHIPTNGSWQWEDGSI LSPNLLTIIEMQKGDCALYASSFKGYIENCSTPNTYICMQRTV (SEQ ID NO:38), or a variant/fragment thereof having at least 70%, 85%, 90%, 95%, 98% or 99% identity to SEQ ID NO:38.

### CD16

In some embodiments, the NK cell receptor is hCD16 (FCGR3A). Therefore, in some embodiments, the NK cell receptor intracellular domain comprises the amino acid sequence KTNIRSSTRDWKDHKFKWRKDPQDK (SEQ ID NO:4), or a variant/fragment thereof having at least 70%, 85%, 90%, 95%, 98% or 99% identity to SEQ ID NO:4. In some embodiments, the TM has the amino acid sequence VSFCLVMVLLFAVDTGLYFSV (SEQ ID NO:5), or a variant/fragment thereof having at least 70%, 85%, 90%, 95%, 98% or 99% identity to SEQ ID NO:5. Therefore, in some embodiments, the CAR comprises the hCD16 TM/IC domains having the amino acid sequence VSFCLVMVLLFAVDTGLYFSVKTNIRSSTRDWKDHKFKWRKDPQDK (SEQ ID NO:6), or a variant/fragment thereof 36, 37, 38, 39, 40, 41, 42, 43, 44, 45, or 46 amino acids in length and having at least 70%, 85%, 90%, 95%, 98% or 99% identity to SEQ ID NO:6 that is capable of integrating into a plasma membrane and binding FcεR1γ when crosslinked. In some embodiments, the extracellular domain has the amino acid sequence MWQLLLPTALLLLVSAGMRTEDLPKAVVFLEPQWYRVLEKDSVTLKCQGAYSPEDNST QWFHNESLISSQASSYFIDAATVDDSGEYRCQTNLSTLSDPVQLEVHIGWLLLQAPRW VFKEEDPIHLRCHSWKNTALHKVTYLQNGKGRKYFHHNSDFYIPKATLKDSGSYFCRG LFGSKNVSSETVNITITQGLAVSTISSFFPPGYQ (SEQ ID NO:39), or a variant/fragment thereof having at least 70%, 85%, 90%, 95%, 98% or 99% identity to SEQ ID NO:39. In some embodiments, hNKG2D has the amino acid sequence MWQLLLPTALLLLVSAGMRTEDLPKAVVFLEPQWYRVLEKDSVTLKCQGAYSPEDNST QWFHNESLISSQASSYFIDAATVDDSGEYRCQTNLSTLSDPVQLEVHIGWLLLQAPRW VFKEEDPIHLRCHSWKNTALHKVTYLQNGKGRKYFHHNSDFYIPKATLKDSGSYFCRG LFGSKNVSSETVNITITQGLAVSTISSFFPPGYQVSFCLVMVLLFAVDTGLYFSVKTNIRS STRDWKDHKFKWRKDPQDK (SEQ ID NO:40), or a variant/fragment thereof having at least 70%, 85%, 90%, 95%, 98% or 99% identity to SEQ ID NO:40.

### NKp30

In some embodiments, the NK cell receptor is hNKp30 (NCR3). Therefore, in some embodiments, the NK cell receptor intracellular domain comprises the amino acid sequence GSTVYYQGKCLTWKGPRRQLPAVVPAPLPPPCGSSAHLLPPVPG (SEQ ID NO:7), or a variant/fragment thereof having at least 70%, 85%, 90%, 95%, 98% or 99% identity to SEQ ID NO:7. In some embodiments, the TM has the amino acid sequence AGTVLLLRAGFYAVSFLSVAV (SEQ ID NO:8), or a variant/fragment thereof having at least 70%, 85%, 90%, 95%, 98% or 99% identity to SEQ ID NO:8. Therefore, in some embodiments, the CAR comprises the hNKp30TM/IC domains having the amino acid sequence AGTVLLLRAGFYAVSFLSVAVGSTVYYQGKCLTWKGPRRQLPAVVPAPLPPPCGSSAH LLPPVPG (SEQ ID NO:9), or a variant/fragment thereof 55, 56, 57, 58, 59, 60, 61, 61, 62, 64, or 65 amino acids in length and having at least 70%, 85%, 90%, 95%, 98% or 99% identity to SEQ ID NO:9 that is capable of integrating into a plasma membrane and binding FcεR1γ when crosslinked. In some embodiments, the extracellular domain has the amino acid sequence MAWMLLLILIMVHPGSCALWVSQPPEIRTLEGSSAFLPCSFNASQGRLAIGSVTWFRDE VVPGKEVRNGTPEFRGRLAPLASSRFLHDHQAELHIRDVRGHDASIYVCRVEVLGLGV GTGNGTRLWEKEHPQLG (SEQ ID NO:41), or a variant/fragment thereof having at least 70%, 85%, 90%, 95%, 98% or 99% identity to SEQ ID NO:41. In some embodiments, hNKG2D has the amino acid sequence MAWMLLLILIMVHPGSCALWVSQPPEIRTLEGSSAFLPCSFNASQGRLAIGSVTWFRDE VVPGKEVRNGTPEFRGRLAPLASSRFLHDHQAELHIRDVRGHDASIYVCRVEVLGLGV GTGNGTRLVVEKEHPQLGAGTVLLLRAGFYAVSFLSVAVGSTVYYQGKCLTWKGPRR QLPAVVPAPLPPPCGSSAHLLPPVPGG (SEQ ID NO:42), or a variant/fragment thereof having at least 70%, 85%, 90%, 95%, 98% or 99% identity to SEQ ID NO:42.

### NKG2C

In some embodiments, the NK cell receptor is hNKG2C. Therefore, in some embodiments, the NK cell receptor intracellular domain comprises the amino acid sequence IPFLEQNNSSPNTRTQKARHCGHCPEEWITYSNSCYYIGKERRTWEESLLACTSKNSSL LSIDNEEEMKFLASILPSSWIGVFRNSSHHPWVTINGLAFKHKIKDSDNAELNCAVLQVN RLKSAQCGSSMIYHCKHKL (SEQ ID NO:10), or a variant/fragment thereof having at least 70%, 85%, 90%, 95%, 98% or 99% identity to SEQ ID NO:10. In some embodiments, the TM has the amino acid sequence LTAEVLGIICIVLMATVLKTIVL (SEQ ID NO: 11), or a variant/fragment thereof having at least 70%, 85%, 90%, 95%, 98% or 99% identity to SEQ ID NO:11. Therefore, in some embodiments, the CAR comprises the hNKG2C TM/IC domains having the amino acid sequence LTAEVLGIICIVLMATVLKTIVLIPFLEQNNSSPNTRTQKARHCGHCPEEWITYSNSCYYIG KERRTWEESLLACTSKNSSLLSIDNEEEMKFLASILPSSWIGVFRNSSHHPWVTINGLAF KHKIKDSDNAELNCAVLQVNRLKSAQCGSSMIYHCKHKL (SEQ ID NO:12), or a variant/fragment thereof 151, 152, 153, 154, 155, 156, 157, 158, 159, 160, or 161 amino acids in length and having at least 70%, 85%, 90%, 95%, 98% or 99% identity to SEQ ID NO:12 that is capable of integrating into a plasma membrane and binding DAP12 when crosslinked. In some embodiments, the extracellular domain has the amino acid sequence MSKQRGTFSEVSLAQDPKRQQRKPKGNKSSISGTEQEIFQVELNLQNPSLNHQGIDKIY DCQGLLPPPEK (SEQ ID NO:43), or a variant/fragment thereof having at least 70%, 85%, 90%, 95%, 98% or 99% identity to SEQ ID NO:43. In some embodiments, hNKG2D has the amino acid sequence MSKQRGTFSEVSLAQDPKRQQRKPKGNKSSISGTEQEIFQVELNLQNPSLNHQGIDKIY DCQGLLPPPEKLTAEVLGI1CIVLMATVLKTIVLIPFLEQNNSSPNTRTQKARHCGHCPEE WITYSNSCYYIGKERRTWEESLLACTSKNSSLLSIDNEEEMKFLASILPSSWIGVFRNSS HHPWVTINGLAFKHKIKDSDNAELNCAVLQVNRLKSAQCGSSMIYHCKHKL (SEQ ID NO:44), or a variant/fragment thereof having at least 70%, 85%, 90%, 95%, 98% or 99% identity to SEQ ID NO:44.

### 2B4

In some embodiments, the NK cell receptor is h2B4. Therefore, in some embodiments, the NK cell receptor intracellular domain comprises the amino acid sequence WRRKRKEKQSETSPKEFLTIYEDVKDLKTRRNHEQEQTFPGGGSTIYSMIQSQSSAPT SQEPAYTLYSLIPSRKSGSRKRNHSPSFNSTIYEVIGKSQPKAQNPARLSRKELENFDV YS (SEQ ID NO:13), or a variant/fragment thereof having at least 70%, 85%, 90%, 95%, 98% or 99% identity to SEQ ID NO:13. In some embodiments, the TM has the amino acid sequence FLVIIVILSALFLGTLACFCV (SEQ ID NO:14), or a variant/fragment thereof having at least 70%, 85%, 90%, 95%, 98% or 99% identity to SEQ ID NO:14. Therefore, in some embodiments, the CAR comprises the h2B4TM/IC domains having the amino acid sequence FLVI VILSALFLGTLACFCVWRRKRKEKQSETSPKEFLTIYEDVKDLKTRRNHEQEQTFP GGGSTIYSMIQSQSSAPTSQEPAYTLYSLIPSRKSGSRKRNHSPSFNSTIYEVIGKSQPK AQNPARLSRKELENFDVYS (SEQ ID NO:15), or a variant/fragment thereof 130, 131, 132, 133, 134, 135, 136, 137, 138, 139, or 140 amino acids in length and having at least 70%, 85%, 90%, 95%, 98% or 99% identity to SEQ ID NO:15 that is capable of signaling when crosslinked. In some embodiments, the extracellular domain has the amino acid sequence MLGQVVTLILLLLLKVYQGKGCQGSADHVVSISGVPLQLQPNSIQTKVDSIAWKKLLPSQ NGFHHILKWENGSLPSNTSNDRFSFIVKNLSLLIKAAQQQDSGLYCLEVTSISGKVQTAT FQVFVFESLLPDKVEKPRLQGQGKILDRGRCQVALSCLVSRDGNVSYAWYRGSKLIQT AGNLTYLDEEVDINGTHTYTCNVSNPVSWESHTLNLTQDCQNAHQEFRFWP (SEQ ID NO:45), or a variant/fragment thereof having at least 70%, 85%, 90%, 95%, 98% or 99% identity to SEQ ID NO:45. In some embodiments, hNKG2D has the amino acid sequence MLGQVVTLILLLLLKVYQGKGCQGSADHVVSISGVPLQLQPNSIQTKVDSIAWKKLLPSQ NGFHHILKWENGSLPSNTSNDRFSFIVKNLSLLIKAAQQQDSGLYCLEVTSISGKVQTAT FQVFVFESLLPDKVEKPRLQGQGKILDRGRCQVALSCLVSRDGNVSYAWYRGSKLIQT AGNLTYLDEEVDINGTHTYTCNVSNPVSWESHTLNLTQDCQNAHQEFRFWPFLVIlVILS ALFLGTLACFCVWRRKRKEKQSETSPKEFLTIYEDVKDLKTRRNHEQEQTFPGGGSTIY SMIQSQSSAPTSQEPAYTLYSLIQPSRKSGSRKRNHSPSFNSTIYEVIGKSQPKAQNPA RLSRKELENFDVYS (SEQ ID NO:46), or a variant/fragment thereof having at least 70%, 85%, 90%, 95%, 98% or 99% identity to SEQ ID NO:46.

### DNAM-1

In some embodiments, the NK cell receptor is hDNAM-1. Therefore, in some embodiments, the NK cell receptor intracellular domain comprises the amino acid sequence NRRRRRERRDLFTESWDTQKAPNNYRSPISTSQPTNQSMDDTREDIYVNYPTFSRRP KTRV (SEQ ID NO:16), or a variant/fragment thereof having at least 70%, 85%, 90%, 95%, 98% or 99% identity to SEQ ID NO:16. In some embodiments, the TM has the amino acid sequence GGTVLLLLFVISITTIIVIFL (SEQ ID NO:17), or a variant/fragment thereof having at least 70%, 85%, 90%, 95%, 98% or 99% identity to SEQ ID NO:17. Therefore, in some embodiments, the CAR comprises the hDNAM TM/IC domains having the amino acid sequence GGTVLLLLFVISITTIIVIFLNRRRRRERRDLFTESWDTQKAPNNYRSPISTSQPTNQSMD DTREDIYVNYPTFSRRPKTRV (SEQ ID NO:18), or a variant/fragment thereof 30, 31, 72, 73, 74, 75, 76, 77. 78, 79, 80, 81, or 82, amino acids in length and having at least 70%, 85%, 90%, 95%, 98% or 99% identity to SEQ ID NO:18 that is capable of signaling when crosslinked. In some embodiments, the extracellular domain has the amino acid sequence MTWPVQAVRWEKIQPRQIDLLTYCNLVHGRNFTSKFPRQIVSNCSHGRWSVIVIPDVTV SDSGLYRCYLQASAGENETFVMRLTVAEGKTDNQYTLFVA (SEQ ID NO:47), or a variant/fragment thereof having at least 70%, 85%, 90%, 95%, 98% or 99% identity to SEQ ID NO:47. In some embodiments, hNKG2D has the amino acid sequence MTWPVQAVRWEKIQPRQIDLLTYCNLVHGRNFTSKFPRQIVSNCSHGRWSVIVIPDVTV SDSGLYRCYLQASAGENETFVMRLTVAEGKTDNQYTLFVAGGTVLLLLFVISITTIIVIFLN RRRRRERRDLFTESWDTQKAPNNYRSPISTSQPTNQSMDDTREDIYVNYPTFSRRPKT RV (SEQ ID NO:48), or a variant/fragment thereof having at least 70%, 85%, 90%, 95%, 98% or 99% identity to SEQ ID NO:48.

### CD137

In some embodiments, the NK cell receptor is hCD137 (TNFRSF9). Therefore, in some embodiments, the NK cell receptor intracellular domain comprises the amino acid sequence KRGRKKLLYIFKQPFMRPVQTTQEEDGCSCRFPEEEEGGCEL (SEQ ID NO:19), or a variant/fragment thereof having at least 70%, 85%, 90%, 95%, 98% or 99% identity to SEQ ID NO:19. In some embodiments, the TM has the amino acid sequence IISFFLALTSTALLFLLFFLTLRFSW (SEQ ID NO:20), or a variant/fragment thereof having at least 70%, 85%, 90%, 95%, 98% or 99% identity to SEQ ID NO:20. Therefore, in some embodiments, the CAR comprises the hCD137 TM/IC domains having the amino acid sequence IISFFLALTSTALLFLLFFLTLRFSVVKRGRKKLLYIFKQPFMRPVQTTQEEDGCSCRFPE EEEGGCEL (SEQ ID NO:21), or a variant/fragment thereof 59, 60, 61, 61, 62, 64, 65, 66, 67, 68, or 69 amino acids in length and having at least 70%, 85%, 90%, 95%, 98% or 99% identity to SEQ ID NO:21 that is capable of signaling when crosslinked. In some embodiments, the extracellular domain has the amino acid sequence MGNSCYNIVATLLLVLNFERTRSLQDPCSNCPAGTFCDNNRNQICSPCPPNSFSSAGG QRTCDICRQCKGVFRTRKECSSTSNAECDCTPGFHCLGAGCSMCEQDCKQGQELTK KGCKDCCFGTFNDQKRGICRPWTNCSLDGKSVLVNGTKERDVVCGPSPADLSPGASS VTPPAPAREPGHSPQ (SEQ ID NO:49), or a variant/fragment thereof having at least 70%, 85%, 90%, 95%, 98% or 99% identity to SEQ ID NO:49. In some embodiments, hNKG2D has the amino acid sequence MGNSCYNIVATLLLVLNFERTRSLQDPCSNCPAGTFCDNNRNQICSPCPPNSFSSAGG QRTCDICRQCKGVFRTRKECSSTSNAECDCTPGFHCLGAGCSMCEQDCKQGQELTK KGCKDCCFGTFNDQKRGICRPWTNCSLDGKSVLVNGTKERDVVCGPSPADLSPGASS VTPPAPAREPGHSPQIISFFLALTSTALLFLLFFLTLRFSVVKRGRKKLLYIFKQPFMRPV QTTQEEDGCSCRFPEEEEGGCEL (SEQ ID NO:50), or a variant/fragment thereof having at least 70%, 85%, 90%, 95%, 98% or 99% identity to SEQ ID NO:50.

### OX-40

In some embodiments, the NK cell receptor is hOX-40. Therefore, in some embodiments, the NK cell receptor intracellular domain comprises the amino acid sequence VSHRYPRIQSIKVQFTEYKKEKGFILTSQKEDEIMKVQNNSVIINCDGFYLISLKGYFSQE VNISLHYQKDEEPLFQLKKVRSVNSLMVASLTYKDKVYLNVTTDNTSLDDFHVNGGELIL IHQNPGEFCVL (SEQ ID NO:22), or a variant/fragment thereof having at least 70%, 85%, 90%, 95%, 98% or 99% identity to SEQ ID NO:22. In some embodiments, the TM has the amino acid sequence LLLVASVIQGLGLLLCFTYICLHFSAL (SEQ ID NO:23), or a variant/fragment thereof having at least 70%, 85%, 90%, 95%, 98% or 99% identity to SEQ ID NO:23. Therefore, in some embodiments, the CAR comprises the hOX-40TM/IC domains having the amino acid sequence LLLVASVIQGLGLLLCFTYICLHFSALVSHRYPRIQSIKVQFTEYKKEKGFILTSQKEDEIM KVQNNSVIINCDGFYLISLKGYFSQEVNISLHYQKDEEPLFQLKKVRSVNSLMVASLTYK DKVYLNVTTDNTSLDDFHVNGGELILIHQNPGEFCVL (SEQ ID NO:24), or a variant/fragment thereof 149, 150, 151, 152, 153, 154, 155, 156, 157, 158, 159 amino acids in length and having at least 70%, 85%, 90%, 95%, 98% or 99% identity to SEQ ID NO:24 that is capable of signaling when crosslinked. In some embodiments, the extracellular domain has the amino acid sequence MERVQPLEENVGNAARPRFERNK (SEQ ID NO:51), or a variant/fragment thereof having at least 70%, 85%, 90%, 95%, 98% or 99% identity to SEQ ID NO:51. In some embodiments, hNKG2D has the amino acid sequence MERVQPLEENVGNAARPRFERNKLLLVASVIQGLGLLLCFTYICLHFSALQVSHRYPRIQ SIKVQFTEYKKEKGFILTSQKEDEIMKVQNNSVIINCDGFYLISLKGYFSQEVNISLHYQK DEEPLFQLKKVRSVNSLMVASLTYKDKVYLNVTTDNTSLDDFHVNGGELILIHQNPGEF CVL (SEQ ID NO:52), or a variant/fragment thereof having at least 70%, 85%, 90%, 95%, 98% or 99% identity to SEQ ID NO:52.

### CD27

In some embodiments, the NK cell receptor is hCD27. Therefore, in some embodiments, the NK cell receptor intracellular domain comprises the amino acid sequence QRRKYRSNKGESPVEPAEPCHYSCPREEEGSTIPIQEDYRKPEPACSP (SEQ ID NO:25), or a variant/fragment thereof having at least 70%, 85%, 90%, 95%, 98% or 99% identity to SEQ ID NO:25. In some embodiments, the TM has the amino acid sequence ILVIFSGMFLVFTLAGALFLH (SEQ ID NO:26), or a variant/fragment thereof having at least 70%, 85%, 90%, 95%, 98% or 99% identity to SEQ ID NO:26. Therefore, in some embodiments, the CAR comprises the hCD27TM/IC domains having the amino acid sequence ILVIFSGMFLVFTLAGALFLHQRRKYRSNKGESPVEPAEPCHYSCPREEEGSTIPIQEDY RKPEPACSP (SEQ ID NO:27), or a variant/fragment 59, 60, 61, 61, 62, 64, 65, 66, 67, 68, or 69 amino acids in length and having at least 70%, 85%, 90%, 95%, 98% or 99% identity to SEQ ID NO:27 that is capable of integrating into a plasma membrane and binding TRAF when crosslinked. In some embodiments, the extracellular domain has the amino acid sequence MNAVIISPSSLQFQKLRPVYTRIAGFKVAPLNKCSLARHFLEPGLLVRNCTITANAECAC RNGWQCRDKECTECDPLPNPSLTARSSQALSPHPQPTHLPYVSEMLEARTAGHMQTL ADFRQLPARTLSTHWPPQRSLCSSDFIR (SEQ ID NO:53), or a variant/fragment thereof having at least 70%, 85%, 90%, 95%, 98% or 99% identity to SEQ ID NO:53. In some embodiments, hNKG2D has the amino acid sequence MNAVIISPSSLQFQKLRPVYTRIAGFKVAPLNKCSLARHFLEPGLLVRNCTITANAECAC RNGWQCRDKECTECDPLPNPSLTARSSQALSPHPQPTHLPYVSEMLEARTAGHMQTL ADFRQLPARTLSTHWPPQRSLCSSDFIRILVIFSGMFLVFTLAGALFLHQRRKYRSNKG ESPVEPAEPCHYSCPREEEGSTIPIQEDYRKPEPACSP (SEQ ID NO:54), or a variant/fragment thereof having at least 70%, 85%, 90%, 95%, 98% or 99% identity to SEQ ID NO:54.

### 2DS5

In some embodiments, the NK cell receptor is Killer cell immunoglobulin-like receptor 2DS5 (KIR2DS5). In some embodiments, the NK cell receptor intracellular (IC) domain comprises the amino acid sequence LLHRWCSNKKNASVMDQGPAGNRTVNREDSDEQDHQEVSYA (SEQ ID NO:28), or a variant/fragment thereof having at least 70%, 85%, 90%, 95%, 98% or 99% identity to SEQ ID NO:28. In some embodiments, the TM has the amino acid sequence VLIGTSWKLPFTILLFFL (SEQ ID NO:29), or or a variant/fragment thereof having at least 70%, 85%, 90%, 95%, 98% or 99% identity to SEQ ID NO:29. Therefore, in some embodiments, the CAR comprises the hNKG2D TM/IC domains having the amino acid sequence VLIGTSVVKLPFTILLFFLLLHRWCSNKKNASVMDQGPAGNRTVNREDSDEQDHQEVS YA (SEQ ID NO:30), or a variant/fragment thereof 50, 51, 52, 53, 54, 55, 56, 57, 58, 59, 60 amino acids in length and having at least 70%, 85%, 90%, 95%, 98% or 99% identity to SEQ ID NO:30 that is capable of integrating into a plasma membrane and binding DAP12 when crosslinked. In some embodiments, the extracellular domain has the amino acid sequence MSLMVISMACVAFFLLQGAWPHEGFRRKPSLLAHPGPLVKSEETVILQCWSDVMFEHF LLHREGTFNHTLRLIGEHIDGVSKGNFSIGRMTQDLAGTYRCYGSVTHSPYQLSAPSDP LDIVITGLYEKPSLSAQPGPTVLAGESVTLSCSSRSSYDMYHLSREGEAHERRLPAGPK VNRTFQADFPLDPATHGGTYRCFGSFRDSPYEWSKSSDPLLVSVTGNSSNSWPSPTE PSSETGNPRHLH (SEQ ID NO:55), or a variant/fragment thereof having at least 70%, 85%, 90%, 95%, 98% or 99% identity to SEQ ID NO:55. In some embodiments, hNKG2D has the amino acid sequence MSLMVISMACVAFFLLQGAWPHEGFRRKPSLLAHPGPLVKSEETVILQCWSDVMFEHF LLHREGTFNHTLRLIGEHIDGVSKGNFSIGRMTQDLAGTYRCYGSVTHSPYQLSAPSDP LDIVITGLYEKPSLSAQPGPTVLAGESVTLSCSSRSSYDMYHLSREGEAHERRLPAGPK VNRTFQADFPLDPATHGGTYRCFGSFRDSPYEWSKSSDPLLVSVTGNSSNSWPSPTE PSSETGNPRHLHVLIGTSVVKLPFTILLFFLLHRWCSNKKNASVMDQGPAGNRTVNRED SDEQDHQEVSYA (SEQ ID NO:56), or a variant/fragment thereof having at least 70%, 85%, 90%, 95%, 98% or 99% identity to SEQ ID NO:56.

### KIR3DS1

In some embodiments, the NK cell receptor is KIR3DS1. In some embodiments, the NK cell receptor intracellular (IC) domain comprises the amino acid sequence HRWCSNKKKCCCNGPRACREQK (SEQ ID NO:31), or a variant/fragment thereof having at least 70%, 85%, 90%, 95%, 98% or 99% identity to SEQ ID NO:31. In some embodiments, the TM has the amino acid sequence ILIGTSWKIPFTILLFFLL (SEQ ID NO:32), or a variant/fragment thereof having at least 70%, 85%, 90%, 95%, 98% or 99% identity to SEQ ID NO:32. Therefore, in some embodiments, the CAR comprises the hNKG2D TM/IC domains having the amino acid sequence ILIGTSVVKIPFTILLFFLLHRWCSNKKKCCCNGPRACREQK (SEQ ID NO:33), or a variant/fragment thereof 32, 33, 34, 35, 36, 37, 38, 39, 40, 41, 42 amino acids in length and having at least 70%, 85%, 90%, 95%, 98% or 99% identity to SEQ ID NO:33 that is capable of integrating into a plasma membrane and binding DAP12 when crosslinked. In some embodiments, the extracellular domain has the amino acid sequence MLLMVVSMACVGLFLVQRAGPHMGGQDKPFLSAWPSAVVPRGGHVTLRCHYRHRFN NFMLYKEDRIHVPIFHGRIFQEGFNMSPVTTAHAGNYTCRGSHPHSPTGWSAPSNPMV IMVTGNHRKPSLLAHPGPLVKSGERVILQCWSDIMFEHFFLHREWISKDPSRLVGQIHD GVSKANFSIGSMMRALAGTYRCYGSVTHTPYQLSAPSDPLDIVVTGLYEKPSLSAQPG PKVQAGESVTLSCSSRSSYDMYHLSREGGAHERRLPAVRKVNRTFQADFPLGPATHG GTYRCFGSFRHSPYEWSDPSDPLLVSVTGNPSSSWPSPTEPSSKSGNLRHLH (SEQ ID NO:57), or a variant/fragment thereof having at least 70%, 85%, 90%, 95%, 98% or 99% identity to SEQ ID NO:57. In some embodiments, hNKG2D has the amino acid sequence MLLMVVSMACVGLFLVQRAGPHMGGQDKPFLSAWPSAVVPRGGHVTLRCHYRHRFN NFMLYKEDRIHVPIFHGRIFQEGFNMSPVTTAHAGNYTCRGSHPHSPTGWSAPSNPMV IMVTGNHRKPSLLAHPGPLVKSGERVILQCWSDIMFEHFFLHREWISKDPSRLVGQIHD GVSKANFSIGSMMRALAGTYRCYGSVTHTPYQLSAPSDPLDIVVTGLYEKPSLSAQPG PKVQAGESVTLSCSSRSSYDMYHLSREGGAHERRLPAVRKVNRTFQADFPLGPATHG GTYRCFGSFRHSPYEWSDPSDPLLVSVTGNPSSSWPSPTEPSSKSGNLRHLHILIGTS VVKIPFTILLFFLLHRWCSNKKKCCCNGPRACREQK (SEQ ID NO:58), or a variant/fragment thereof having at least 70%, 85%, 90%, 95%, 98% or 99% identity to SEQ ID NO:58.

### NKp80/KLRF1

In some embodiments, the NK cell receptor is NKp80/KLRF1. In some embodiments, the NK cell receptor intracellular (IC) domain comprises the amino acid sequence MQDEERYMTLNVQSKKRSSAQTSQLTFKDYSVTLHWYK (SEQ ID NO:34), or a variant/fragment thereof having at least 70%, 85%, 90%, 95%, 98% or 99% identity to SEQ ID NO:34. In some embodiments, the TM has the amino acid sequence ILLGISGTVNGILTLTLISLI (SEQ ID NO:35), or a variant/fragment thereof having at least 70%, 85%, 90%, 95%, 98% or 99% identity to SEQ ID NO:35. Therefore, in some embodiments, the CAR comprises the hNKG2D TM/IC domains having the amino acid sequence MQDEERYMTLNVQSKKRSSAQTSQLTFKDYSVTLHWYKILLGISGTVNGILTLTLISLI (SEQ ID NO:36), or a variant/fragment thereof 49, 50, 51, 52, 53, 54, 55, 56, 57, 58, 59 acids in length and having at least 70%, 85%, 90%, 95%, 98% or 99% identity to SEQ ID NO:36 that is capable of integrating signaling when crosslinked. In some embodiments, the extracellular domain has the amino acid sequence LLVSQGVLLKCQKGSCSNATQYEDTGDLKVNNGTRRNISNKDLCASRSADQTVLCQSE WLKYQGKCYWFSNEMKSWSDSYVYCLERKSHLLIIHDQLEMAFIQKNLRQLNYVWIGL NFTSLKMTWTWVDGSPIDSKIFFIKGPAKENSCAAIKESKIFSETCSSVFKWICQY (SEQ ID NO:59), or a variant/fragment thereof having at least 70%, 85%, 90%, 95%, 98% or 99% identity to SEQ ID NO:59. In some embodiments, hNKG2D has the amino acid sequence MQDEERYMTLNVQSKKRSSAQTSQLTFKDYSVTLHWYKILLGISGTVNGILTLTLISLILL VSQGVLLKCQKGSCSNATQYEDTGDLKVNNGTRRNISNKDLCASRSADQTVLCQSEW LKYQGKCYWFSNEMKSWSDSYVYCLERKSHLLIIHDQLEMAFIQKNLRQLNYVWIGLNF TSLKMTWTWVDGSPIDSKIFFIKGPAKENSCAAIKESKIFSETCSSVFKWICQY (SEQ ID NO:60), or a variant/fragment thereof having at least 70%, 85%, 90%, 95%, 98% or 99% identity to SEQ ID NO:60.

### Binding Domain

The binding domain is in some embodiments an antibody fragment that specifically binds a target molecule on a cell, such as tumor associated antigen (TAA). For example, the antigen binding domain can be a Fab or a single-chain variable fragment (scFv) of an antibody that specifically binds the target molecule. The binding domain is in some embodiments an aptamer that specifically binds the tar target molecule. For example, the binding domain can be a peptide aptamer selected from a random sequence pool based on its ability to bind the target molecule. The binding domain can also be a natural ligand of the target molecule, or a variant and/or fragment thereof capable of binding the target molecule.

### CD33

For example, an scFv that selectively binds CD33 is described in US2020/0223920, which is incorporated by reference in its entirety for the description of these antibodies and their sequences. For example, in some embodiments, the anti-CD33 region of the disclosed antibody or CAR is derived from hybridoma 27A3, 33G3, 36C2, 6A11, 35D5, 38G5, or combinations thereof. In some embodiments, the anti-CD33 region (e.g. scFv) can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GFTFSNYG (SEQ ID NO:61), GYTFTSYW (SEQ ID NO:62), or GFSLSRYS (SEQ ID NO:63), wherein the CDR2 sequence of the V_{H} domain comprises the amino acid sequence ISSGGGDT (SEQ ID NO:64), IHPSDSET (SEQ ID NO:65), or IWGGGYT (SEQ ID NO:66), wherein the CDR3 sequence of the V_{H} domain comprises the amino acid sequence ARDYGGTWDYFDY (SEQ ID NO:67), AREEGQLGHGGAMDY (SEQ ID NO:68), or ARYIDSSGYDY (SEQ ID NO:69), wherein the CDR1 sequence of the V_{L} comprises the amino acid sequence QDISKY (SEQ ID NO:70), QTVNDD (SEQ ID NO:71), SSVSY (SEQ ID NO:72), or ENIYSY (SEQ ID NO:73), wherein the CDR2 sequence of the V_{L} domain comprises the amino acid sequence YTS, YVS, DTS, or NAK, wherein the CDR3 sequence of the V_{L} domain comprises the amino acid sequence QQGDTFPWT (SEQ ID NO:78), QQDYSSPYT (SEQ ID NO:79), QQWSSNPLT (SEQ ID NO:80), or QHHYGTPYT (SEQ ID NO:81), or any combination thereof.

Therefore, in some embodiments, the anti-CD33
scFv V_{H} domain comprises the amino acid sequence
(SEQ ID NO: 82)
EVKLVESGGGLVKPGASLKLSCAASGFTFSNYGMSWVRQTSDKRLEWV
ASISSGGGDTYYPDNVKGRFTISRENAKNTLYLQMSSLNSEDTALYYC
ARDYGGTWDYFDYWGQGTTLTVSS,
(SEQ ID NO: 83)
QVQLQQPGAELVRPGVSVKLSCKASGYTFTSYWMNWVKQRPGQGLEWI
GMIHPSDSETRLNQKFKDKAILTVDKSSSTAYMQLSSPTSEDSAVYYC
AREEGQLGHGGAMDYWGQGTSVTVSS,
or
(SEQ ID NO: 84)
QVQLKESGPGLVAPSQSLSITCTVSGFSLSRYSVHWVRQPPGKGLEWL
GMIWGGGYTDYNSALKSRLSISKDNSKSQVFLKMNSLQTDDTAMYYCA
RYIDSSGYDYWGQGTTLTVSS.
In some embodiments, the anti-CD33 scFv V_{L}
domain comprises the amino acid sequence
(SEQ ID NO: 85)
DIQMTQTTSSLSASLGDRVTISCRASQDISKYLNWYQQKPDGTVKLLI
YYTSRLHSGVPSRFSGSGSGTDYSLTISNLEQEDIATYFCQQGDTFPW
TFGGGTKLEIK,
(SEQ ID NO: 86)
SIVMTQTPKFLLVSAGDRVTITCKASQTVNDDVAWYQQKPGQSPKLLI
YYVSNRHTGVPDRFTGSGYGTDFTFTISTVQAEDLAVYFCQQDYSSPY
TFGGGTKLEIK,
(SEQ ID NO: 87)
QIVLTQSPAIMSASPGEKVTMTCSASSSVSYMHWYQQKSGTSPKRWIY
DTSKLASGVPARFSGSGSGTSYSLTISSMEAEDAATYYCQQWSSNPLT
FGAGTKLELK,
or
(SEQ ID NO: 88)
DIQMTQSPASLSASVGETVTITCRASENIYSYLAWYQQKQGKSPQLLV
YNAKTLAEGVPSRFSGSGSGTQFSLKINSLQPEDFGSYYCQHHYGTPY
TFGGGTKLEIK.

The heavy and light chains are preferably separated by a linker. Suitable linkers for scFv antibodies are known in the art. In some embodiments, the linker comprises the amino acid sequence GGGGSGGGGSGGGGS (SEQ ID NO:89).

In some embodiments, the anti-CD33 scFv comprises
the amino acid sequence:
(SEQ ID NO: 90, 6A11HC1_LC)
EVKLVESGGGLVKPGASLKLSCAASGFTFSNYGMSWVRQTSDKRLEWVA
SISSGGGDTYYPDNVKGRFTISRENAKNTLYLQMSSLNSEDTALYYCAR
DYGGTWDYFDYWGQGTTLTVSSGGGGSGGGGSGGGGSDIQMTQTTSSLS
ASLGDRVTISCRASQDISKYLNWYQQKPDGTVKLLIYYTSRLHSGVPSR
FSGSGSGTDYSLTISNLEQEDIATYFCQQGDTFPWTFGGGTKLEIK.
In some embodiments, the anti-CD33 scFv comprises
the amino acid sequence:
(SEQ ID NO: 91, 6A11HC2_LC)
QVQLQQPGAELVRPGVSVKLSCKASGYTFTSYWMNWVKQRPGQGLEWIG
MIHPSDSETRLNQKFKDKAILTVDKSSSTAYMQLSSPTSEDSAVYYCAR
EEGQLGHGGAMDYWGQGTSVTVSSGGGGSGGGGSGGGGSDIQMTQTTSS
LSASLGDRVTISCRASQDISKYLNWYQQKPDGTVKLLIYYTSRLHSGVP
SRFSGSGSGTDYSLTISNLEQEDIATYFCQQGDTFPWTFGGGTKLEIK.
In some embodiments, the anti-CD33 scFv comprises
the amino acid sequence:
(SEQ ID NO: 92, 27A3HC_LC1)
QVQLKESGPGLVAPSQSLSITCTVSGFSLSRYSVHWVRQPPGKGLEWLG
MIWGGGYTDYNSALKSRLSISKDNSKSQVFLKMNSLQTDDTAMYYCARY
IDSSGYDYWGQGTTLTVSSGGGGSGGGGSGGGGSSIVMTQTPKFLLVSA
GDRVTITCKASQTVNDDVAWYQQKPGQSPKLLIYYVSNRHTGVPDRFTG
SGYGTDFTFTISTVQAEDLAVYFCQQDYSSPYTFGGGTKLEIK.
In some embodiments, the anti-CD33 scFv comprises
the amino acid sequence:
(SEQ ID NO: 93, 27A3HC_LC2)
QVQLKESGPGLVAPSQSLSITCTVSGFSLSRYSVHWVRQPPGKGLEWLG
MIWGGGYTDYNSALKSRLSISKDNSKSQVFLKMNSLQTDDTAMYYCARY
IDSSGYDYWGQGTTLTVSSGGGGSGGGGSGGGGSQIVLTQSPAIMSASP
GEKVTMTCSASSSVSYMHWYQQKSGTSPKRWIYDTSKLASGVPARFSGS
GSGTSYSLTISSMEAEDAATYYCQQWSSNPLTFGAGTKLELK.
In some embodiments, the anti-CD33 scFv comprises
the amino acid sequence:
(SEQ ID NO: 94, 27A3HC_LC3)
QVQLKESGPGLVAPSQSLSITCTVSGFSLSRYSVHWVRQPPGKGLEWLG
MIWGGGYTDYNSALKSRLSISKDNSKSQVFLKMNSLQTDDTAMYYCARY
IDSSGYDYWGQGTTLTVSSGGGGSGGGGSGGGGSDIQMTQSPASLSASV
GETVTITCRASENIYSYLAWYQQKQGKSPQLLVYNAKTLAEGVPSRFSG
SGSGTQFSLKINSLQPEDFGSYYCQHHYGTPYTFGGGTKLEIK.

### CD123

For example, an scFv that selectively binds CD123 is described in 2020/0165348, which is incorporated by reference in its entirety for the description of these antibodies and their sequences. For example, in some embodiments, the anti-CD123 scFv is derived from hybridoma 3F5, 4E10, 12H5, 15A12, 17E7, 12H11, or combinations thereof. In some embodiments, the anti-CD123 scFv can comprise a variable heavy (V_{H}) domain having CDR1, CDR2 and CDR3 sequences and a variable light (V_{L}) domain having CDR1, CDR2 and CDR3 sequences. For example, in some embodiments, the CDR1 sequence of the V_{H} domain comprises the amino acid sequence GYTFTDYN (SEQ ID NO:95), CDR2 sequence of the V_{H} domain comprises the amino acid sequence INPNNGGT (SEQ ID NO:96), CDR3 sequence of the V_{H} domain comprises the amino acid sequence ARKGYGGNYDYFDY (SEQ ID NO:97), CDR1 sequence of the V_{L} comprises the amino acid sequence QSIGTS (SEQ ID NO:98), CDR2 sequence of the V_{L} domain comprises the amino acid sequence YASx (SEQ ID NO:99), and CDR3 sequence of the V_{L} domain comprises the amino acid sequence QQSNSWPYT (SEQ ID NO:100).

In some embodiments, the CDR1 sequence of the V_{H} domain comprises the amino acid sequence GFNIKDTY (SEQ ID NO:101) or GFSLSTYGMG (SEQ ID NO:102), the CDR2 sequence of the V_{H} domain comprises the amino acid sequence IDPANGNT (SEQ ID NO:103) or IYWDDDK (SEQ ID NO:104), the CDR3 sequence of the V_{H} domain comprises the amino acid sequence ALYYYGGSLDY (SEQ ID NO:105) or AQSLIYDGYYGFAY (SEQ ID NO:106), the CDR1 sequence of the V_{L} comprises the amino acid sequence QSLLYSGNQKNY (SEQ ID NO:107), the CDR2 sequence of the V_{L} domain comprises the amino acid sequence WASx (SEQ ID NO:108), and the CDR3 sequence of the V_{L} domain comprises the amino acid sequence QQYYSYPRT (SEQ ID NO:109).

In some embodiments, the CDR1 sequence of the V_{H} domain comprises the amino acid sequence GYTFTYYG (SEQ ID NO:110), the CDR2 sequence of the V_{H} domain comprises the amino acid sequence INTYSGVP (SEQ ID NO:111), the CDR3 sequence of the V_{H} domain comprises the amino acid sequence ARWIYYSDLYGMDY (SEQ ID NO: 112), the CDR1 sequence of the V_{L} comprises the amino acid sequence QSIVHSNGDTY (SEQ ID NO:113), the CDR2 sequence of the V_{L} domain comprises the amino acid sequence KVSx (SEQ ID NO:114), and the CDR3 sequence of the V_{L}domain comprises the amino acid sequence FQGSHVPWT (SEQ ID NO:115).

In some embodiments, the CDR1 sequence of the V_{H} domain comprises the amino acid sequence GYTFSSYW (SEQ ID NO: 116) or GYTLTTYL (SEQ ID NO: 117), the CDR2 sequence of the V_{H} domain comprises the amino acid sequence INPSSGYT (SEQ ID NO:118) or INPNSGSS (SEQ ID NO:119), the CDR3 sequence of the V_{H} domain comprises the amino acid sequence ARDGNYDHWYFDV (SEQ ID NO:120) or AIRHYGGSLFDY (SEQ ID NO:121), the CDR1 sequence of the V_{L} comprises the amino acid sequence QDINSY (SEQ ID NO:122) or QSLLNSRTRKNY (SEQ ID NO:123), the CDR2 sequence of the V_{L} domain comprises the amino acid sequence WAS or RAN, and the CDR3 sequence of the V_{L} domain comprises the amino acid sequence LQYDELLT (SEQ ID NO:126) or EQSYNLFT (SEQ ID NO:127).

In some embodiments, the some embodiments, the CDR1 sequence of the V_{H} domain comprises the amino acid sequence GYTFTDYN (SEQ ID NO:128), GFNIKDTY (SEQ ID NO:129), GFSLSTYGMG (SEQ ID NO:130), GYTFTYYG (SEQ ID NO:131), GYTFSSYW (SEQ ID NO:132), or GYTLTTYL (SEQ ID NO:133); the CDR2 sequence of the V_{H} domain comprises the amino acid sequence INPNNGGT (SEQ ID NO: 96), IDPANGNT (SEQ ID NO:103), IYWDDDK (SEQ ID NO:104), INTYSGVP (SEQ ID NO: 111), INPSSGYT (SEQ ID NO:134), or INPNSGSS (SEQ ID NO:135); the CDR3 sequence of the V_{H} domain comprises the amino acid sequence ARKGYGGNYDYFDY (SEQ ID NO:97), ALYYYGGSLDY (SEQ ID NO:105), AQSLIYDGYYGFAY (SEQ ID NO:106), ARWIYYSDLYGMDY (SEQ ID NO: 112), ARDGNYDHWYFDV (SEQ ID NO:136), or AIRHYGGSLFDY (SEQ ID NO:137); the CDR1 sequence of the V_{L}comprises the amino acid sequence QSIGTS (SEQ ID NO:98), QSLLYSGNQKNY (SEQ ID NO:107), QSIVHSNGDTY (SEQ ID NO:113), QDINSY (SEQ ID NO:122), or QSLLNSRTRKNY (SEQ ID NO:138); the CDR2 sequence of the V_{L} domain comprises the amino acid sequence YASx, WASx, KVSx, or RAN; the CDR3 sequence of the V_{L}domain comprises the amino acid sequence QQSNSWPYT, QQYYSYPRT, FQGSHVPWT, LQYDELLT (SEQ ID NO:140), or EQSYNLFT (SEQ ID NO:141); or any combination thereof.

Therefore, in some embodiments, the anti-CD123 scFv V_{H} domain comprises the
amino acid sequence:
(SEQ ID NO: 142, 3F5HC1)
EVQLQQSGPELVKPGSSVKISCKASGYTFTDYNMDWVKQSHGKSLEWIGTINPNNGG
TSYNQKFKGKATLTVDKSSSTAYMELRSLTSEDSAVYYCARKGYGGNYDYFDYWGQG
TTLTVSS,
(SEQ ID NO: 143, 12H1HC1)
EVQLQQSGAELVKPGASVKLSCTASGFNIKDTYMHWVKQRPEQGLEWIGRIDPANGN
TIYASKFQGKATITADTSSNTAYMQLSSLTSGDTAVYYCALYYYGGSLDYWGQGTTLTVSS,
(SEQ ID NO: 144, 12H1HC2)
QVTLKESGPGILQPSQTLSLTCSFSGFSLSTYGMGVSWIRQPSGKGLEWLAHIYWDDD
KRYNPSLKSRLTISKDTSNNQVFLKITSVDTADTATYYCAQSLIYDGYYGFAYWGQGTL
VTVSA,
(SEQ ID NO: 145, 12H2HC1)
QIQLVQSGPELKKPGETVKISCKASGYTFTYYGMNWVKQAPGKGLEWMGWINTYSGV
PTYADDFKGRFAFSLETSVSTAYLQINNLKNEDTATYFCARWIYYSDLYGMDYWGQGT
SVTVSS,
(SEQ ID NO: 146, 15A12HC1)
QVQLQQSGAELAKPGASVKMSCKASGYTFSSYWMHWLKQRPGQGLEWIGYINPSSG
YTNYNQKFKDKATLTADKSSSTAYMQLSSLTSEDSAVYYCARDGNYDHWYFDVWGTG
TTVTVSS,
or
(SEQ ID NO: 147, 15A12HC2)
QVQLQQPGAELVRPGASVKMSCKASGYTLTTYLMDWVKQRLGQGFEWIGNINPNSGS
SNYNEKFKGKAKLTVDKSSSTAYMQLSSLTSEDSAVYYCAIRHYGGSLFDYWGQGTTL
TVSS.
In some embodiments, the anti-CD123 scFv V_{L} domain comprises the amino acid
sequence:
(SEQ ID NO: 148; 3F5LC1)
DILLTQSPAILSVSPGERVSFSCRASQSIGTSIHWYQQRTNGSPRLLIKYASESISGIPSR
FSGSGSGTDFTLSINSVESEDIADYYCQQSNSWPYTFGGGTKLEIK,
(SEQ ID NO: 149, 12H1LC1)
DIVMSQSPSSLAVSVGERVTMSCKSSQSLLYSGNQKNYLAWYQQKPGQSPKLLIYWA
STRESGVPDRFTGSGSGTDFTLTISSVKAEDLAVYYCQQYYSYPRTFGGGTKLEIK,
(SEQ ID NO: 150, 12H2LC)
DVLMTQSPLSLPVSLGDQASISCRSSQSIVHSNGDTYLEWYLQKPGQSPKLLIYKVSNR
FSGVPDRFSGSGSGTDFTLKISRVEAEDLGVYHCFQGSHVPWTFGGGTKLEIK,
(SEQ ID NO: 151, 15A12LC1)
DIKMTQSPSSMYASLGERVTITCKASQDINSYLSWFQQKPGKSPKTLIYRANRLVDGVP
SRFSGSGSGQDYSLTISSLEYEDMGIYYCLQYDELLTFGAGTKLELK,
or
(SEQ ID NO: 152, 15A12LC2)
DIVMSQSPSSLAVSAGERVTMSCRSSQSLLNSRTRKNYLAWYQQKPGQSPKLLIYWAS
TRESGVPDRFSGSGSGTDFTLTISSVQAEDLAVYYCEQSYNLFTFGSGTKLEIK.
In some embodiments, the anti-CD123 scFv comprises an amino acid sequence:
(SEQ ID NO: 153, 3F5HC1_LC)
EVQLQQSGPELVKPGSSVKISCKASGYTFTDYNMDWVKQSHGKSLEWIGTINPNNGG
TSYNQKFKGKATLTVDKSSSTAYMELRSLTSEDSAVYYCARKGYGGNYDYFDYWGQG
TTLTVSSGGGGSGGGGSGGGGSDILLTQSPAILSVSPGERVSFSCRASQSIGTSIHWY
QQRTNGSPRLLIKYASESISGIPSRFSGSGSGTDFTLSINSVESEDIADYYCQQSNSWP
YTFGGGTKLEIK.
In some embodiments, the anti-CD123 scFv comprises an amino acid sequence:
(SEQ ID NO: 154, 12H1HC1_LC1)
EVQLQQSGAELVKPGASVKLSCTASGFNIKDTYMHWVKQRPEQGLEWIGRIDPANGN
TIYASKFQGKATITADTSSNTAYMQLSSLTSGDTAVYYCALYYYGGSLDYWGQGTTLTV
SSGGGGSGGGGSGGGGSDIVMSQSPSSLAVSVGERVTMSCKSSQSLLYSGNQKNYL
AWYQQKPGQSPKLLIYWASTRESGVPDRFTGSGSGTDFTLTISSVKAEDLAVYYCQQY
YSYPRTFGGGTKLEIK.
In some embodiments, the anti-CD123 scFv comprises an amino acid sequence:
(SEQ ID NO: 155, 12H1HC2_LC1)
QVTLKESGPGILQPSQTLSLTCSFSGFSLSTYGMGVSWIRQPSGKGLEWLAHIYWDDD
KRYNPSLKSRLTISKDTSNNQVFLKITSVDTADTATYYCAQSLIYDGYYGFAYWGQGTL
VTVSAGGGGSGGGGSGGGGSDIVMSQSPSSLAVSVGERVTMSCKSSQSLLYSGNQK
NYLAWYQQKPGQSPKLLIYWASTRESGVPDRFTGSGSGTDFTLTISSVKAEDLAVYYC
QQYYSYPRTFGGGTKLEIK.
In some embodiments, the anti-CD123 scFv comprises an amino acid sequence:
(SEQ ID NO: 156, 12H2HC1_LC1)
QIQLVQSGPELKKPGETVKISCKASGYTFTYYGMNWVKQAPGKGLEWMGWINTYSGV
PTYADDFKGRFAFSLETSVSTAYLQINNLKNEDTATYFCARWIYYSDLYGMDYWGQGT
SVTVSSGGGGSGGGGSGGGGSDVLMTQSPLSLPVSLGDQASISCRSSQSIVHSNGDT
YLEWYLQKPGQSPKLLIYKVSNRFSGVPDRFSGSGSGTDFTLKISRVEAEDLGVYHCF
QGSHVPWTFGGGTKLEIK.
In some embodiments, the anti-CD123 scFv comprises an amino acid sequence:
(SEQ ID NO: 157, 15A12HC1_LC1)
QVQLQQSGAELAKPGASVKMSCKASGYTFSSYWMHWLKQRPGQGLEWIGYINPSSG
YTNYNQKFKDKATLTADKSSSTAYMQLSSLTSEDSAVYYCARDGNYDHWYFDVWGTG
TTVTVSSGGGGSGGGGSGGGGSDIKMTQSPSSMYASLGERVTITCKASQDINSYLSW
FQQKPGKSPKTLIYRANRLVDGVPSRFSGSGSGQDYSLTISSLEYEDMGIYYCLQYDEL
LTFGAGTKLELK.
In some embodiments, the anti-CD123 scFv comprises an amino acid sequence:
(SEQ ID NO: 158, 15A12HC1_LC2)
QVQLQQSGAELAKPGASVKMSCKASGYTFSSYWMHWLKQRPGQGLEWIGYINPSSG
YTNYNQKFKDKATLTADKSSSTAYMQLSSLTSEDSAVYYCARDGNYDHWYFDVWGTG
TTVTVSSGGGGSGGGGSGGGGSDIVMSQSPSSLAVSAGERVTMSCRSSQSLLNSRT
RKNYLAWYQQKPGQSPKLLIYWASTRESGVPDRFSGSGSGTDFTLTISSVQAEDLAVY
YCEQSYNLFTFGSGTKLEIK.
In some embodiments, the anti-CD123 scFv comprises an amino acid sequence:
(SEQ ID NO: 159, 15A12HC2_LC1)
QVQLQQPGAELVRPGASVKMSCKASGYTLTTYLMDWVKQRLGQGFEWIGNINPNSGS
SNYNEKFKGKAKLTVDKSSSTAYMQLSSLTSEDSAVYYCAIRHYGGSLFDYWGQGTTL
TVSSGGGGSGGGGSGGGGSDIKMTQSPSSMYASLGERVTITCKASQDINSYLSWFQQ
KPGKSPKTLIYRANRLVDGVPSRFSGSGSGQDYSLTISSLEYEDMGIYYCLQYDELLTF
GAGTKLELK.
In some embodiments, the anti-CD123 scFv comprises an amino acid sequence:
(SEQ ID NO: 160, 15A12HC2_LC2)
QVQLQQPGAELVRPGASVKMSCKASGYTLTTYLMDWVKQRLGQGFEWIGNINPNSGS
SNYNEKFKGKAKLTVDKSSSTAYMQLSSLTSEDSAVYYCAIRHYGGSLFDYWGQGTTL
TVSSGGGGSGGGGSGGGGSDIVMSQSPSSLAVSAGERVTMSCRSSQSLLNSRTRKN
YLAWYQQKPGQSPKLLIYWASTRESGVPDRFSGSGSGTDFTLTISSVQAEDLAVYYCE
QSYNLFTFGSGTKLEIK.

### CD99

For example, an scFv that selectively binds CD99 is described in US20200397882, which is incorporated by reference in its entirety for the description of these antibodies and their sequences.

In some embodiments, the anti-CD99 region of the disclosed antibody or CAR is derived from hybridoma 1H3, 4C5, 9G12, 3C7, 2F11, 4D5, 4F4, 6A10, or combinations thereof. In some embodiments, the anti-CD99 region (e.g. scFv) can comprise a variable heavy (V_{H}) domain having CDR1, CDR2 and CDR3 sequences and a variable light (V_{L}) domain having CDR1, CDR2 and CDR3 sequences.

In some embodiments, the CDR1 sequence of the V_{H} domain comprises the amino acid sequence GFDIKDTY (SEQ ID NO:161), TYAMY (SEQ ID NO:162), TFWM (SEQ ID NO:163), or TFWMQ (SEQ ID NO:164); the CDR2 sequence of the V_{H} domain comprises the amino acid sequence IDPANGDT (SEQ ID NO:165), RIRSKVNNYATYYADSVKDRFT (SEQ ID NO:166), or TIYPGDDDTRYTQKFKGRAT (SEQ ID NO:167); the CDR3 sequence of the V_{H} domain comprises the amino acid sequence ARRGGLS (SEQ ID NO:168), DPMDY (SEQ ID NO:169), or SGYERGPYYFDS (SEQ ID NO:170), or SGYERGPYYF (SEQ ID NO:171); the CDR1 sequence of the V_{L} comprises the amino acid sequence GNIHNY (SEQ ID NO:172), GSSKSLLHSNGNTYLY (SEQ ID NO:173), KSSQSLLCRSNQKNYLA (SEQ ID NO:174), or KSSQSLLYRSNQKNYLA (SEQ ID NO:175); the CDR2 sequence of the V_{L} domain comprises the amino acid sequence NAK, RVSNLAS (SEQ ID NO:177), or WASTRES (SEQ ID NO:178); and the CDR3 sequence of the V_{L} domain comprises the amino acid sequence QHFWSTPWT (SEQ ID NO:179), MQHLEYPYT (SEQ ID NO:180), or QQYYSYPLT (SEQ ID NO:181).

Therefore, in some embodiments, the anti-CD99 V_{H} domain comprises the amino
acid sequence
(SEQ ID NO: 182, 1H3H7)
EVQLQQSGAELVKPGASVKLSCTASGFDIKDTYIHWVKQRPEQGLEWIGRIDPANGDT
RYDPEFQGKASLTADTSSNTAYLQFSNLTSEDTAVYYCARRGGLSWGQGTTLTVSS.
Therefore, in some embodiments, the anti-CD99 V_{H} domain comprises the amino
acid sequence
(SEQ ID NO: 183, 4C5E2)
EVQLEESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVSS.
Therefore, in some embodiments, the anti-CD99 V_{H} domain comprises the amino
acid sequence
(SEQ ID NO: 184, 4C5H10)
EVQLVESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVSS.
Therefore, in some embodiments, the anti-CD99 V_{H} domain comprises the amino
acid sequence
(SEQ ID NO: 185, 9G12C9)
QVQLQQSGAELARPGASVKLSCKASGYTFTTFWMQWVKQRPGQGLEWIGTIYPGDD
DTRYTQKFKGRATLTADKSSTTAYMQLSNLSSEDSAVYYCARSGYERGPYYFDSWGQ
GTTLTVSS.
Therefore, in some embodiments, the anti-CD99 V_{H} domain comprises the amino
acid sequence
(SEQ ID NO: 186, 9G12G6 HB1)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSS.
In some embodiments, the anti-CD99 V_{H} domain comprises the amino acid
sequence
(SEQ ID NO: 187, 9G12G6 HB3)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSS.
In some embodiments, the anti-CD99 V_{L} domain comprises the amino acid
sequence
(SEQ ID NO: 188, 1H3H9)
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIK.
In some embodiments, the anti-CD99 V_{L} domain comprises the amino acid
sequence
(SEQ ID NO: 189, 1H3H7 LC1)
GNSWSHSLRSLSVTIGQPASISCKSSQSLLDGNGKTYLNWLLQRPGQSPKRLLYLVSK
LDSGVPDRFTGSGSGTDFTLKISRVEAEDLGIYYCWQGTHFPRTFGGGTKLEIK.
In some embodiments, the anti-CD99 V_{L} domain comprises the amino acid
sequence
(SEQ ID NO: 190, 1H3H7 LC2)
GNSWRHSPRSLSVTIGQPASISCKSSQSLLDGNGKTYLNWLLQRPGQSPKRLLYLVSK
LDSGVPDRFTGSGSGTDFTLKISRVEAEDLGIYYCWQGTHFPRTFGGGTKLEIK.
In some embodiments, the anti-CD99 V_{L} domain comprises the amino acid
sequence
(SEQ ID NO: 191, 4C5E2)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIK.
In some embodiments, the anti-CD99 V_{L} domain comprises the amino acid
sequence
(SEQ ID NO: 192, 4C5H10)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIK.
In some embodiments, the anti-CD99 V_{L} domain comprises the amino acid
sequence
(SEQ ID NO: 193, 9G12C9)
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELK.
In some embodiments, the anti-CD99 V_{L} domain comprises the amino acid
sequence
(SEQ ID NO: 194, 9G12G6)
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELK
In some embodiments, the anti-CD99 scFv comprises an amino acid sequence:
(SEQ ID NO: 195)
EVQLQQSGAELVKPGASVKLSCTASGFDIKDTYIHWVKQRPEQGLEWIGRIDPANGDT
RYDPEFQGKASLTADTSSNTAYLQFSNLTSEDTAVYYCARRGGLSWGQGTTLTVSSG
GGGSGGGGSGGGGSDIQMTQSPASLSASVGETVTITCRASGNIHNYLAWYQQKQGK
SPQLLVYNAKTLADGVPSRFSGSGSGTQYSLKINSLQPEDFGSYYCQHFWSTPWTFG
GGTKLEIK.
In some embodiments, the anti-CD99 scFv comprises an amino acid sequence:
(SEQ ID NO: 196)
EVQLQQSGAELVKPGASVKLSCTASGFDIKDTYIHWVKQRPEQGLEWIGRIDPANGDT
RYDPEFQGKASLTADTSSNTAYLQFSNLTSEDTAVYYCARRGGLSWGQGTTLTVSSG
GGGSGGGGSGGGGSGNSWSHSLRSLSVTIGQPASISCKSSQSLLDGNGKTYLNWLL
QRPGQSPKRLLYLVSKLDSGVPDRFTGSGSGTDFTLKISRVEAEDLGIYYCWQGTHFP
RTFGGGTKLEIK.
In some embodiments, the anti-CD99 scFv comprises an amino acid sequence:
(SEQ ID NO: 197)
EVQLQQSGAELVKPGASVKLSCTASGFDIKDTYIHWVKQRPEQGLEWIGRIDPANGDT
RYDPEFQGKASLTADTSSNTAYLQFSNLTSEDTAVYYCARRGGLSWGQGTTLTVSSG
GGGSGGGGSGGGGSGNSWRHSPRSLSVTIGQPASISCKSSQSLLDGNGKTYLNWLL
QRPGQSPKRLLYLVSKLDSGVPDRFTGSGSGTDFTLKISRVEAEDLGIYYCWQGTHFP
RTFGGGTKLEIK.
In some embodiments, the anti-CD99 scFv comprises an amino acid sequence:
(SEQ ID NO: 198)
EVQLQQSGAELVKPGASVKLSCTASGFDIKDTYIHWVKQRPEQGLEWIGRIDPANGDT
RYDPEFQGKASLTADTSSNTAYLQFSNLTSEDTAVYYCARRGGLSWGQGTTLTVSSG
GGGSGGGGSGGGGSDIVMTQAAPSVPVTPGESVSISCGSSKSLLHSNGNTYLYWFLQ
RPGQSPQLLIYRVSNLASGVPDRFSGSGSGTAFTLRISRVEAEDVGVYYCMQHLEYPY
TFGGGTRLEIK.
In some embodiments, the anti-CD99 scFv comprises an amino acid sequence:
(SEQ ID NO: 199)
EVQLQQSGAELVKPGASVKLSCTASGFDIKDTYIHWVKQRPEQGLEWIGRIDPANGDT
RYDPEFQGKASLTADTSSNTAYLQFSNLTSEDTAVYYCARRGGLSWGQGTTLTVSSG
GGGSGGGGSGGGGSDTVMSQSPSSLAVSVGEKITMSCKSSQSLLCRSNQKNYLAWY
QQKPGQSPKQLIYWASTRESGVPDRFTGSGSGTDFTLTISSVKAEDLAVYYCQQYYSY
PLTFGAGTKLELK.
In some embodiments, the anti-CD99 scFv comprises an amino acid sequence:
(SEQ ID NO: 200)
EVQLQQSGAELVKPGASVKLSCTASGFDIKDTYIHWVKQRPEQGLEWIGRIDPANGDT
RYDPEFQGKASLTADTSSNTAYLQFSNLTSEDTAVYYCARRGGLSWGQGTTLTVSSG
GGGSGGGGSGGGGSDTVMSQSPSSLAVSVGEKITMSCKSSQSLLYRSNQKNYLAWY
QQKPGQSPKQLIYWASTRESGVPDRFTGSGSGTDFTLTISSVKAEDLAVYYCQQYYSY
PLTFGAGTKLELK.
In some embodiments, the anti-CD99 scFv comprises an amino acid sequence:
(SEQ ID NO: 201)
EVQLEESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDIQMTQSPASLSASVGETVTITCRASGNIHNYLAWYQQKQ
GKSPQLLVYNAKTLADGVPSRFSGSGSGTQYSLKINSLQPEDFGSYYCQHFWSTPWT
FGGGTKLEIK.
In some embodiments, the anti-CD99 scFv comprises an amino acid sequence:
(SEQ ID NO: 202)
EVQLEESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSGNSWSHSLRSLSVTIGQPASISCKSSQSLLDGNGKTYLNW
LLQRPGQSPKRLLYLVSKLDSGVPDRFTGSGSGTDFTLKISRVEAEDLGIYYCWQGTHF
PRTFGGGTKLEIK.
In some embodiments, the anti-CD99 scFv comprises an amino acid sequence:
(SEQ ID NO: 203)
EVQLEESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSGNSWRHSPRSLSVTIGQPASISCKSSQSLLDGNGKTYLNW
LLQRPGQSPKRLLYLVSKLDSGVPDRFTGSGSGTDFTLKISRVEAEDLGIYYCWQGTHF
PRTFGGGTKLEIK.
In some embodiments, the anti-CD99 scFv comprises an amino acid sequence:
(SEQ ID NO: 204)
EVQLEESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDIVMTQAAPSVPVTPGESVSISCGSSKSLLHSNGNTYLYWF
LQRPGQSPQLLIYRVSNLASGVPDRFSGSGSGTAFTLRISRVEAEDVGVYYCMQHLEY
PYTFGGGTRLEIK.
In some embodiments, the anti-CD99 scFv comprises an amino acid sequence:
(SEQ ID NO: 205)
EVQLEESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDTVMSQSPSSLAVSVGEKITMSCKSSQSLLCRSNQKNYLA
WYQQKPGQSPKQLIYWASTRESGVPDRFTGSGSGTDFTLTISSVKAEDLAVYYCQQY
YSYPLTFGAGTKLELK.
In some embodiments, the anti-CD99 scFv comprises an amino acid sequence:
(SEQ ID NO: 206)
EVQLEESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDTVMSQSPSSLAVSVGEKITMSCKSSQSLLYRSNQKNYLA
WYQQKPGQSPKQLIYWASTRESGVPDRFTGSGSGTDFTLTISSVKAEDLAVYYCQQY
YSYPLTFGAGTKLELK.
In some embodiments, the anti-CD99 scFv comprises an amino acid sequence:
(SEQ ID NO: 207)
EVQLVESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDIQMTQSPASLSASVGETVTITCRASGNIHNYLAWYQQKQ
GKSPQLLVYNAKTLADGVPSRFSGSGSGTQYSLKINSLQPEDFGSYYCQHFWSTPWT
FGGGTKLEIK.
In some embodiments, the anti-CD99 scFv comprises an amino acid sequence:
(SEQ ID NO: 208)
EVQLVESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSGNSWSHSLRSLSVTIGQPASISCKSSQSLLDGNGKTYLNW
LLQRPGQSPKRLLYLVSKLDSGVPDRFTGSGSGTDFTLKISRVEAEDLGIYYCWQGTHF
PRTFGGGTKLEIK.
In some embodiments, the anti-CD99 scFv comprises an amino acid sequence:
(SEQ ID NO: 209)
EVQLVESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSGNSWRHSPRSLSVTIGQPASISCKSSQSLLDGNGKTYLNW
LLQRPGQSPKRLLYLVSKLDSGVPDRFTGSGSGTDFTLKISRVEAEDLGIYYCWQGTHF
PRTFGGGTKLEIK.
In some embodiments, the anti-CD99 scFv comprises an amino acid sequence:
(SEQ ID NO: 210)
EVQLVESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDIVMTQAAPSVPVTPGESVSISCGSSKSLLHSNGNTYLYWF
LQRPGQSPQLLIYRVSNLASGVPDRFSGSGSGTAFTLRISRVEAEDVGVYYCMQHLEY
PYTFGGGTRLEIK.
In some embodiments, the anti-CD99 scFv comprises an amino acid sequence:
(SEQ ID NO: 211)
EVQLVESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDTVMSQSPSSLAVSVGEKITMSCKSSQSLLCRSNQKNYLA
WYQQKPGQSPKQLIYWASTRESGVPDRFTGSGSGTDFTLTISSVKAEDLAVYYCQQY
YSYPLTFGAGTKLELK.
In some embodiments, the anti-CD99 scFv comprises an amino acid sequence:
(SEQ ID NO: 212)
EVQLVESGGGLVQPKGSLKLSCAASGFTFNTYAMYWVCQAPGKGLKWVARIRSKVNN
YATYYADSVKDRFTISRDDSQNMLFLHMNNLKTEDTAIYFCVRDPMDYWGQGISVTVS
SGGGGSGGGGSGGGGSDTVMSQSPSSLAVSVGEKITMSCKSSQSLLYRSNQKNYLA
WYQQKPGQSPKQLIYWASTRESGVPDRFTGSGSGTDFTLTISSVKAEDLAVYYCQQY
YSYPLTFGAGTKLELK.
In some embodiments, the anti-CD99 scFv comprises an amino acid sequence:
(SEQ ID NO: 213)
QVQLQQSGAELARPGASVKLSCKASGYTFTTFWMQWVKQRPGQGLEWIGTIYPGDD
DTRYTQKFKGRATLTADKSSTTAYMQLSNLSSEDSAVYYCARSGYERGPYYFDSWGQ
GTTLTVSSGGGGSGGGGSGGGGSDIQMTQSPASLSASVGETVTITCRASGNIHNYLA
WYQQKQGKSPQLLVYNAKTLADGVPSRFSGSGSGTQYSLKINSLQPEDFGSYYCQHF
WSTPWTFGGGTKLEIK.
In some embodiments, the anti-CD99 scFv comprises an amino acid sequence:
(SEQ ID NO: 214)
QVQLQQSGAELARPGASVKLSCKASGYTFTTFWMQWVKQRPGQGLEWIGTIYPGDD
DTRYTQKFKGRATLTADKSSTTAYMQLSNLSSEDSAVYYCARSGYERGPYYFDSWGQ
GTTLTVSSGGGGSGGGGSGGGGSGNSWSHSLRSLSVTIGQPASISCKSSQSLLDGNG
KTYLNWLLQRPGQSPKRLLYLVSKLDSGVPDRFTGSGSGTDFTLKISRVEAEDLGIYYC
WQGTHFPRTFGGGTKLEIK.
In some embodiments, the anti-CD99 scFv comprises an amino acid sequence:
(SEQ ID NO: 215)
QVQLQQSGAELARPGASVKLSCKASGYTFTTFWMQWVKQRPGQGLEWIGTIYPGDD
DTRYTQKFKGRATLTADKSSTTAYMQLSNLSSEDSAVYYCARSGYERGPYYFDSWGQ
GTTLTVSSGGGGSGGGGSGGGGSGNSWRHSPRSLSVTIGQPASISCKSSQSLLDGN
GKTYLNWLLQRPGQSPKRLLYLVSKLDSGVPDRFTGSGSGTDFTLKISRVEAEDLGIYY
CWQGTHFPRTFGGGTKLEIK.
In some embodiments, the anti-CD99 scFv comprises an amino acid sequence:
(SEQ ID NO: 216)
QVQLQQSGAELARPGASVKLSCKASGYTFTTFWMQWVKQRPGQGLEWIGTIYPGDD
DTRYTQKFKGRATLTADKSSTTAYMQLSNLSSEDSAVYYCARSGYERGPYYFDSWGQ
GTTLTVSSGGGGSGGGGSGGGGSDIVMTQAAPSVPVTPGESVSISCGSSKSLLHSNG
NTYLYWFLQRPGQSPQLLIYRVSNLASGVPDRFSGSGSGTAFTLRISRVEAEDVGVYY
CMQHLEYPYTFGGGTRLEIK.
In some embodiments, the anti-CD99 scFv comprises an amino acid sequence:
(SEQ ID NO: 217)
QVQLQQSGAELARPGASVKLSCKASGYTFTTFWMQWVKQRPGQGLEWIGTIYPGDD
DTRYTQKFKGRATLTADKSSTTAYMQLSNLSSEDSAVYYCARSGYERGPYYFDSWGQ
GTTLTVSSGGGGSGGGGSGGGGSDTVMSQSPSSLAVSVGEKITMSCKSSQSLLCRS
NQKNYLAWYQQKPGQSPKQLIYWASTRESGVPDRFTGSGSGTDFTLTISSVKAEDLAV
YYCQQYYSYPLTFGAGTKLELK.
In some embodiments, the anti-CD99 scFv comprises an amino acid sequence:
(SEQ ID NO: 218)
QVQLQQSGAELARPGASVKLSCKASGYTFTTFWMQWVKQRPGQGLEWIGTIYPGDD
DTRYTQKFKGRATLTADKSSTTAYMQLSNLSSEDSAVYYCARSGYERGPYYFDSWGQ
GTTLTVSSGGGGSGGGGSGGGGSDTVMSQSPSSLAVSVGEKITMSCKSSQSLLYRSN
QKNYLAWYQQKPGQSPKQLIYWASTRESGVPDRFTGSGSGTDFTLTISSVKAEDLAVY
YCQQYYSYPLTFGAGTKLELK.
In some embodiments, the anti-CD99 scFv comprises an amino acid sequence:
(SEQ ID NO: 219)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDIQMTQSPASLSASVGETVTITCRASGNIHNYLAW
YQQKQGKSPQLLVYNAKTLADGVPSRFSGSGSGTQYSLKINSLQPEDFGSYYCQHFW
STPWTFGGGTKLEIK.
In some embodiments, the anti-CD99 scFv comprises an amino acid sequence:
(SEQ ID NO: 220)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSGNSWSHSLRSLSVTIGQPASISCKSSQSLLDGNGK
TYLNWLLQRPGQSPKRLLYLVSKLDSGVPDRFTGSGSGTDFTLKISRVEAEDLGIYYCW
QGTHFPRTFGGGTKLEIK.
In some embodiments, the anti-CD99 scFv comprises an amino acid sequence:
(SEQ ID NO: 221)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSGNSWRHSPRSLSVTIGQPASISCKSSQSLLDGNGK
TYLNWLLQRPGQSPKRLLYLVSKLDSGVPDRFTGSGSGTDFTLKISRVEAEDLGIYYCW
QGTHFPRTFGGGTKLEIK.
In some embodiments, the anti-CD99 scFv comprises an amino acid sequence:
(SEQ ID NO: 222)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDIVMTQAAPSVPVTPGESVSISCGSSKSLLHSNGN
TYLYWFLQRPGQSPQLLIYRVSNLASGVPDRFSGSGSGTAFTLRISRVEAEDVGVYYC
MQHLEYPYTFGGGTRLEIK.
In some embodiments, the anti-CD99 scFv comprises an amino acid sequence:
(SEQ ID NO: 223)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDIVMTQAAPSVPVTPGESVSISCGSSKSLLHSNGN
TYLYWFLQRPGQSPQLLIYRVSNLASGVPDRFSGSGSGTAFTLRISRVEAEDVGVYYC
MQHLEYPYTFGGGTRLEIK.
In some embodiments, the anti-CD99 scFv comprises an amino acid sequence:
(SEQ ID NO: 224)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDTVMSQSPSSLAVSVGEKITMSCKSSQSLLCRSN
QKNYLAWYQQKPGQSPKQLIYWASTRESGVPDRFTGSGSGTDFTLTISSVKAEDLAVY
YCQQYYSYPLTFGAGTKLELK.
In some embodiments, the anti-CD99 scFv comprises an amino acid sequence:
(SEQ ID NO: 225)
DVKLQESGAELARPGASVKLSCKASGYTFTTFWMQRVKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDTVMSQSPSSLAVSVGEKITMSCKSSQSLLYRSNQ
KNYLAWYQQKPGQSPKQLIYWASTRESGVPDRFTGSGSGTDFTLTISSVKAEDLAVYY
CQQYYSYPLTFGAGTKLELK.
In some embodiments, the anti-CD99 scFv comprises an amino acid sequence:
(SEQ ID NO: 226)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDIQMTQSPASLSASVGETVTITCRASGNIHNYLAW
YQQKQGKSPQLLVYNAKTLADGVPSRFSGSGSGTQYSLKINSLQPEDFGSYYCQHFW
STPWTFGGGTKLEIK.
In some embodiments, the anti-CD99 scFv comprises an amino acid sequence:
(SEQ ID NO: 227)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSGNSWSHSLRSLSVTIGQPASISCKSSQSLLDGNGK
TYLNWLLQRPGQSPKRLLYLVSKLDSGVPDRFTGSGSGTDFTLKISRVEAEDLGIYYCW
QGTHFPRTFGGGTKLEIK.
In some embodiments, the anti-CD99 scFv comprises an amino acid sequence:
(SEQ ID NO: 228)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSGNSWRHSPRSLSVTIGQPASISCKSSQSLLDGNGK
TYLNWLLQRPGQSPKRLLYLVSKLDSGVPDRFTGSGSGTDFTLKISRVEAEDLGIYYCW
QGTHFPRTFGGGTKLEIK.
In some embodiments, the anti-CD99 scFv comprises an amino acid sequence:
(SEQ ID NO: 229)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDIVMTQAAPSVPVTPGESVSISCGSSKSLLHSNGN
TYLYWFLQRPGQSPQLLIYRVSNLASGVPDRFSGSGSGTAFTLRISRVEAEDVGVYYC
MQHLEYPYTFGGGTRLEIK.
In some embodiments, the anti-CD99 scFv comprises an amino acid sequence:
(SEQ ID NO: 230)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDIVMTQAAPSVPVTPGESVSISCGSSKSLLHSNGN
TYLYWFLQRPGQSPQLLIYRVSNLASGVPDRFSGSGSGTAFTLRISRVEAEDVGVYYC
MQHLEYPYTFGGGTRLEIK.
In some embodiments, the anti-CD99 scFv comprises an amino acid sequence:
(SEQ ID NO: 231)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDTVMSQSPSSLAVSVGEKITMSCKSSQSLLCRSN
QKNYLAWYQQKPGQSPKQLIYWASTRESGVPDRFTGSGSGTDFTLTISSVKAEDLAVY
YCQQYYSYPLTFGAGTKLELK.
In some embodiments, the anti-CD99 scFv comprises an amino acid sequence:
(SEQ ID NO: 232)
QVQLKESGAELARPGASVKLSCKASGYTFTTFWMQWAKQRPGQGLEWIGTIYPGDDD
TRYTQKFKGRATLTADKSSTTAYMQLSNLSSEDSAVYYCARSGYERGPYYFDSWGQG
TTLTVSSGGGGSGGGGSGGGGSDTVMSQSPSSLAVSVGEKITMSCKSSQSLLYRSNQ
KNYLAWYQQKPGQSPKQLIYWASTRESGVPDRFTGSGSGTDFTLTISSVKAEDLAVYY
CQQYYSYPLTFGAGTKLELK.
In some embodiments, the anti-CD99 scFv comprises an amino acid sequence:
(SEQ ID NO: 233)
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIKGGGGSGGG
GSGGGGSEVQLQQSGAELVKPGASVKLSCTASGFDIKDTYIHWVKQRPEQGLEWIGRI
DPANGDTRYDPEFQGKASLTADTSSNTAYLQFSNLTSEDTAVYYCARRGGLSWGQGT
TLTVSS.
In some embodiments, the anti-CD99 scFv comprises an amino acid sequence:
(SEQ ID NO: 234)
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIKGGGGSGGG
GSGGGGSEVQLEESGGGLVQPKGSLKLSCAASGFTFNTYAMYWVCQAPGKGLKWVA
RIRSKVNNYATYYADSVKDRFTISRDDSQNMLFLHMNNLKTEDTAIYFCVRDPMDYWG
QGISVTVSS.
In some embodiments, the anti-CD99 scFv comprises an amino acid sequence:
(SEQ ID NO: 235)
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIKGGGGSGGG
GSGGGGSEVQLVESGGGLVQPKGSLKLSCAASGFTFNTYAMYWVCQAPGKGLKWVA
RIRSKVNNYATYYADSVKDRFTISRDDSQNMLFLHMNNLKTEDTAIYFCVRDPMDYWG
QGISVTVSS.
In some embodiments, the anti-CD99 scFv comprises an amino acid sequence:
(SEQ ID NO: 236)
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIKGGGGSGGG
GSGGGGSQVQLQQSGAELARPGASVKLSCKASGYTFTTFWMQWVKQRPGQGLEWI
GTIYPGDDDTRYTQKFKGRATLTADKSSTTAYMQLSNLSSEDSAVYYCARSGYERGPY
YFDSWGQGTTLTVSS.
In some embodiments, the anti-CD99 scFv comprises an amino acid sequence:
(SEQ ID NO: 237)
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIKGGGGSGGG
GSGGGGSDVKLQESGAELARPGASVKLSCKASGYTFTTFWMQRVKQRPGQGLEWIG
TIYPGDDDTRYTQKFKGRATLTADKSSTTAYMQLSNLSSEDSAVYYCARSGYERGPYY
FDSWGQGTTLTVSS.
In some embodiments, the anti-CD99 scFv comprises an amino acid sequence:
(SEQ ID NO: 238)
DIQMTQSPASLSASVGETVTITCRASGNIHNYLAWYQQKQGKSPQLLVYNAKTLADGV
PSRFSGSGSGTQYSLKINSLQPEDFGSYYCQHFWSTPWTFGGGTKLEIKGGGGSGGG
GSGGGGSQVQLKESGAELARPGASVKLSCKASGYTFTTFWMQWAKQRPGQGLEWIG
TIYPGDDDTRYTQKFKGRATLTADKSSTTAYMQLSNLSSEDSAVYYCARSGYERGPYY
FDSWGQGTTLTVSS.
In some embodiments, the anti-CD99 scFv comprises an amino acid sequence:
(SEQ ID NO: 239)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIKGGGGS
GGGGSGGGGSEVQLQQSGAELVKPGASVKLSCTASGFDIKDTYIHWVKQRPEQGLE
WIGRIDPANGDTRYDPEFQGKASLTADTSSNTAYLQFSNLTSEDTAVYYCARRGGLSW
GQGTTLTVSS.
In some embodiments, the anti-CD99 scFv comprises an amino acid sequence:
(SEQ ID NO: 240)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIKGGGGS
GGGGSGGGGSEVQLEESGGGLVQPKGSLKLSCAASGFTFNTYAMYWVCQAPGKGLK
WVARIRSKVNNYATYYADSVKDRFTISRDDSQNMLFLHMNNLKTEDTAIYFCVRDPMD
YWGQGISVTVSS.
In some embodiments, the anti-CD99 scFv comprises an amino acid sequence:
(SEQ ID NO: 241)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIKGGGGS
GGGGSGGGGSEVQLVESGGGLVQPKGSLKLSCAASGFTFNTYAMYWVCQAPGKGLK
WVARIRSKVNNYATYYADSVKDRFTISRDDSQNMLFLHMNNLKTEDTAIYFCVRDPMD
YWGQGISVTVSS.
In some embodiments, the anti-CD99 scFv comprises an amino acid sequence:
(SEQ ID NO: 242)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIKGGGGS
GGGGSGGGGSQVQLQQSGAELARPGASVKLSCKASGYTFTTFWMQWVKQRPGQGL
EWIGTIYPGDDDTRYTQKFKGRATLTADKSSTTAYMQLSNLSSEDSAVYYCARSGYER
GPYYFDSWGQGTTLTVSS.
In some embodiments, the anti-CD99 scFv comprises an amino acid sequence:
(SEQ ID NO: 243)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIKGGGGS
GGGGSGGGGSDVKLQESGAELARPGASVKLSCKASGYTFTTFWMQRVKQRPGQGLE
WIGTIYPGDDDTRYTQKFKGRATLTADKSSTTAYMQLSNLSSEDSAVYYCARSGYERG
PYYFDSWGQGTTLTVSS.
In some embodiments, the anti-CD99 scFv comprises an amino acid sequence:
(SEQ ID NO: 244)
DIVMTQAAPSVPVTPGESVSISCGSSKSLLHSNGNTYLYWFLQRPGQSPQLLIYRVSNL
ASGVPDRFSGSGSGTAFTLRISRVEAEDVGVYYCMQHLEYPYTFGGGTRLEIKGGGGS
GGGGSGGGGSQVQLKESGAELARPGASVKLSCKASGYTFTTFWMQWAKQRPGQGL
EWIGTIYPGDDDTRYTQKFKGRATLTADKSSTTAYMQLSNLSSEDSAVYYCARSGYER
GPYYFDSWGQGTTLTVSS.
In some embodiments, the anti-CD99 scFv comprises an amino acid sequence:
(SEQ ID NO: 245)
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSEVQLQQSGAELVKPGASVKLSCTASGFDIKDTYIHWVKQRPEQ
GLEWIGRIDPANGDTRYDPEFQGKASLTADTSSNTAYLQFSNLTSEDTAVYYCARRGG
LSWGQGTTLTVSS.
In some embodiments, the anti-CD99 scFv comprises an amino acid sequence:
(SEQ ID NO: 246)
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSEVQLEESGGGLVQPKGSLKLSCAASGFTFNTYAMYWVCQAPG
KGLKWVARIRSKVNNYATYYADSVKDRFTISRDDSQNMLFLHMNNLKTEDTAIYFCVRD
PMDYWGQGISVTVSS.
In some embodiments, the anti-CD99 scFv comprises an amino acid sequence:
(SEQ ID NO: 247)
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSEVQLVESGGGLVQPKGSLKLSCAASGFTFNTYAMYWVCQAPG
KGLKWVARIRSKVNNYATYYADSVKDRFTISRDDSQNMLFLHMNNLKTEDTAIYFCVRD
PMDYWGQGISVTVSS.
In some embodiments, the anti-CD99 scFv comprises an amino acid sequence:
(SEQ ID NO: 248)
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSQVQLQQSGAELARPGASVKLSCKASGYTFTTFWMQWVKQRPG
QGLEWIGTIYPGDDDTRYTQKFKGRATLTADKSSTTAYMQLSNLSSEDSAVYYCARSG
YERGPYYFDSWGQGTTLTVSS.
In some embodiments, the anti-CD99 scFv comprises an amino acid sequence:
(SEQ ID NO: 249)
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSDVKLQESGAELARPGASVKLSCKASGYTFTTFWMQRVKQRPG
QGLEWIGTIYPGDDDTRYTQKFKGRATLTADKSSTTAYMQLSNLSSEDSAVYYCARSG
YERGPYYFDSWGQGTTLTVSS.
In some embodiments, the anti-CD99 scFv comprises an amino acid sequence:
(SEQ ID NO: 250)
DTVMSQSPSSLAVSVGEKITMSCKSSQSLLCRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSQVQLKESGAELARPGASVKLSCKASGYTFTTFWMQWAKQRPG
QGLEWIGTIYPGDDDTRYTQKFKGRATLTADKSSTTAYMQLSNLSSEDSAVYYCARSG
YERGPYYFDSWGQGTTLTVSS.
In some embodiments, the anti-CD99 scFv comprises an amino acid sequence:
(SEQ ID NO: 251)
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSEVQLQQSGAELVKPGASVKLSCTASGFDIKDTYIHWVKQRPEQ
GLEWIGRIDPANGDTRYDPEFQGKASLTADTSSNTAYLQFSNLTSEDTAVYYCARRGG
LSWGQGTTLTVSS.
In some embodiments, the anti-CD99 scFv comprises an amino acid sequence:
(SEQ ID NO: 252)
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSEVQLEESGGGLVQPKGSLKLSCAASGFTFNTYAMYWVCQAPG
KGLKWVARIRSKVNNYATYYADSVKDRFTISRDDSQNMLFLHMNNLKTEDTAIYFCVRD
PMDYWGQGISVTVSS.
In some embodiments, the anti-CD99 scFv comprises an amino acid sequence:
(SEQ ID NO: 253)
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSEVQLVESGGGLVQPKGSLKLSCAASGFTFNTYAMYWVCQAPG
KGLKWVARIRSKVNNYATYYADSVKDRFTISRDDSQNMLFLHMNNLKTEDTAIYFCVRD
PMDYWGQGISVTVSS.
In some embodiments, the anti-CD99 scFv comprises an amino acid sequence:
(SEQ ID NO: 254)
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSQVQLQQSGAELARPGASVKLSCKASGYTFTTFWMQWVKQRPG
QGLEWIGTIYPGDDDTRYTQKFKGRATLTADKSSTTAYMQLSNLSSEDSAVYYCARSG
YERGPYYFDSWGQGTTLTVSS.
In some embodiments, the anti-CD99 scFv comprises an amino acid sequence:
(SEQ ID NO: 255)
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSDVKLQESGAELARPGASVKLSCKASGYTFTTFWMQRVKQRPG
QGLEWIGTIYPGDDDTRYTQKFKGRATLTADKSSTTAYMQLSNLSSEDSAVYYCARSG
YERGPYYFDSWGQGTTLTVSS.
In some embodiments, the anti-CD99 scFv comprises an amino acid sequence:
(SEQ ID NO: 256)
DTVMSQSPSSLAVSVGEKITMSCKSSQSLLYRSNQKNYLAWYQQKPGQSPKQLIYWA
STRESGVPDRFTGSGSGTDFTLTISSVKAEDLAVYYCQQYYSYPLTFGAGTKLELKGG
GGSGGGGSGGGGSQVQLKESGAELARPGASVKLSCKASGYTFTTFWMQWAKQRPG
QGLEWIGTIYPGDDDTRYTQKFKGRATLTADKSSTTAYMQLSNLSSEDSAVYYCARSG
YERGPYYFDSWGQGTTLTVSS.

### CLEC12A

For example, an scFv that selectively binds CLEC12A is described in US20200345779, which is incorporated by reference in its entirety for the description of these antibodies and their sequences. For example, in some embodiments, the anti-CLEC12A region of the disclosed antibody or CAR is derived from hybridoma 1F3, 1F8, 1G3, 2A10, 3F12, 4E3, 4E10, 5B2, 5F10, 6C7, 9A2, 11C7, 11H1, 12D6, or combinations thereof. In some embodiments, the anti-CLEC12A region (e.g. scFv) can comprise a variable heavy (V_{H}) domain having CDR1, CDR2 and CDR3 sequences and a variable light (V_{L}) domain having CDR1, CDR2 and CDR3 sequences.

In some embodiments, the CDR1 sequence of the V_{H} domain comprises the amino acid sequence GFTFSSFA (SEQ ID NO:257) SFAVS (SEQ ID NO:258), or SHDMS (SEQ ID NO:259); the CDR2 sequence of the V_{H} domain comprises the amino acid sequence ISSGGAYT (SEQ ID NO:260) orTISSGGAYTFYKDSVKGRFT (SEQ ID NO:261), or YISGGGTNIYYSDTVKGRFT (SEQ ID NO:262); the CDR3 sequence of the V_{H} domain comprises the amino acid sequence ARHSGYDGYYLYAMDY (SEQ ID NO:263), HSGYDGYYLYAMDY (SEQ ID NO:264), or PNYNYGGSWFAY (SEQ ID NO:265); the CDR1 sequence of the V_{L} comprises the amino acid sequence SSVHY (SEQ ID NO:266), ASSSVHYMH (SEQ ID NO:267), or SASSSVHYMH (SEQ ID NO:268); the CDR2 sequence of the V_{L} domain comprises the amino acid sequence DTS or DTSKLAS (SEQ ID NO:270); and the CDR3 sequence of the V_{L} domain comprises the amino acid sequence QQWTSNPPT (SEQ ID NO:271).

In some embodiments, the anti-CLEC12A V_{H} domain comprises the amino acid
sequence
(SEQ ID NO: 272, 1F3H8)
ELILVESGGGLVKPGGSLKLSCAVSGFTFSSFAMSWVRQTPEKRLEWVATISSGG
AYTFYKDSVKGRFTISRDNAKNTLYLQMSSLRSEDSAMYYCARHSGYDGYYLYAM
DYWGQGTSVTVSS.
Therefore, in some embodiments, the anti-CLEC12A V_{H} domain is encoded by
the nucleic acid sequence
(SEQ ID NO: 273, 1F3H8)
GAACTAATACTGGTGGAGTCTGGGGGAGGCTTAGTGAAGCCTGGAGGGTCCCTGA
AACTCTCCTGTGCAGTCTCTGGATTCACTTTCAGTTCCTTTGCCATGTCTTGGGT
TCGCCAGACTCCGGAGAAGAGGCTGGAGTGGGTCGCAACCATTAGTAGTGGTGGA
GCTTACACCTTCTATAAAGACAGTGTGAAGGGGCGATTCACCATCTCCAGAGACA
ATGCCAAGAATACCCTGTACCTGCAAATGAGCAGTCTGAGGTCTGAGGACTCGGC
CATGTATTACTGTGCAAGACATAGCGGCTATGATGGTTACTACCTCTATGCTATG
GACTACTGGGGTCAAGGAACCTCAGTCACCGTCTCCTCA.
In some embodiments, the anti-CLEC12A V_{H} domain comprises the amino acid
sequence
(SEQ ID NO: 274, 1F3A10)
GVQCELILVESGGGLVKPGGSLKLSCAVSGFTFSSFAVSWVRQTPEKRLEWVATI
SSGGAYTFYKDSVKGRFTISRDNAKNTLYLQMSSLRSEDSAMYYCARHSGYDGYY
LYAMDYWGQGTSVTVSS.
Therefore, in some embodiments, the anti-CLEC12A V_{H} domain is encoded by
the nucleic acid sequence
(SEQ ID NO: 275, 1F3A10)
GGTGTCCAGTGTGAACTAATACTGGTGGAGTCTGGGGGAGGCTTAGTGAAGCCTG
GAGGGTCCCTGAAACTCTCCTGTGCAGTCTCTGGATTCACTTTCAGTTCCTTTGC
CGTGTCCTGGGTTCGCCAGACTCCGGAGAAGAGGCTGGAGTGGGTCGCAACCATT
AGTAGTGGTGGAGCTTACACCTTCTATAAAGACAGTGTGAAGGGGCGATTCACCA
TCTCCAGAGACAATGCCAAGAATACCCTGTACCTGCAAATGAGCAGTCTGAGGTC
TGAGGACTCGGCCATGTATTACTGTGCAAGACATAGCGGCTATGATGGTTACTAC
CTCTATGCTATGGACTACTGGGGTCAAGGAACCTCAGTCACCGTCTCCTCA.
In some embodiments, the anti-CLEC12A V_{H} domain comprises the amino acid
sequence
(SEQ ID NO: 276, 1F3F3)
EVQLEESGGGLVQPGGSLKVSCAVSGLAFSSHDMSWVRQTPEKRLEWVAYISGGG
TNIYYSDTVKGRFTISRDNAKNTLYLQMSSLKSEDTAIYYCARPNYNYGGSWFAY
WGQGTLVTVSA.
Therefore, in some embodiments, the anti-CLEC12A V_{H} domain is encoded by
the nucleic acid sequence
(SEQ ID NO: 277, 1F3F3)
GAGGTGCAGCTGGAGGAGTCTGGGGGAGGCTTAGTGCAGCCGGGAGGGTCCCTGA
AAGTCTCCTGTGCAGTTTCCGGACTCGCTTTCAGCAGCCATGACATGTCTTGGGT
TCGCCAGACTCCGGAGAAGCGGCTGGAGTGGGTCGCATACATTAGTGGAGGTGGT
ACTAATATCTATTATTCAGACACTGTGAAGGGCCGATTCACCATCTCCAGAGACA
ATGCCAAGAACACCCTGTACCTGCAAATGAGCAGTCTGAAGTCTGAAGACACAGC
CATTTATTACTGTGCAAGACCCAATTATAATTACGGCGGTTCCTGGTTTGCTTAC
TGGGGCCAAGGGACTCTGGTCACTGTCTCTGCA.
In some embodiments, the anti-CLEC12A V_{L} domain comprises the amino acid
sequence
(SEQ ID NO: 278, 1F3H8, 1F3F3, 1F3A10)
QIVLTQSPEIMSASPGEKVTMTCSASSSVHYMHWYQQKSGTSPKRWIYDTSKLAS
GVPGRFSGSGSGTSYSLTISSMESEDAATYYCQQWTSNPPTFGGGTKLEIK.
Therefore, in some embodiments, the anti-CLEC12A V_{L} domain is encoded by
the nucleic acid sequence
(SEQ ID NO: 279, 1F3H8, 1F3F3, 1F3A10)
CAAATTGTTCTCACCCAGTCTCCAGAAATCATGTCTGCATCTCCAGGGGAGAAGG
TCACCATGACCTGCAGTGCCAGCTCAAGTGTACATTACATGCACTGGTACCAGCA
GAAGTCAGGCACCTCCCCCAAAAGATGGATTTATGACACATCCAAACTGGCTTCT
GGAGTCCCTGGTCGCTTCAGTGGCAGTGGGTCTGGGACCTCTTACTCTCTCACAA
TCAGCAGCATGGAGTCTGAAGATGCTGCCACTTATTACTGCCAGCAGTGGACTAG
TAACCCACCCACGTTCGGAGGGGGGACCAAGCTGGAAATTAAACG.
In some embodiments, the anti-CLEC12A scFv comprises an amino acid
sequence:
(SEQ ID NO: 280, 1F3H8)
ELILVESGGGLVKPGGSLKLSCAVSGFTFSSFAMSWVRQTPEKRLEWVATISSGG
AYTFYKDSVKGRFTISRDNAKNTLYLQMSSLRSEDSAMYYCARHSGYDGYYLYAM
DYWGQGTSVTVSSGGGGGGGGSGGGGSQIVLTQSPEIMSASPGEKVTMTCSASSS
VHYMHWYQQKSGTSPKRWIYDTSKLASGVPGRFSGSGSGTSYSLTISSMESEDAA
TYYCQQWTSNPPTFGGGTKLEIK.
In some embodiments, the anti-CLEC12A scFv comprises an amino acid
sequence:
(SEQ ID NO: 281, 1F3A10)
GVQCELILVESGGGLVKPGGSLKLSCAVSGFTFSSFAVSWVRQTPEKRLEWVATI
SSGGAYTFYKDSVKGRFTISRDNAKNTLYLQMSSLRSEDSAMYYCARHSGYDGYY
LYAMDYWGQGTSVTVSSGGGGSGGGGSGGGGSQIVLTQSPEIMSASPGEKVTMTC
SASSSVHYMHWYQQKSGTSPKRWIYDTSKLASGVPGRFSGSGSGTSYSLTISSME
SEDAATYYCQQWTSNPPTFGGGTKLEIK.
In some embodiments, the anti-CLEC12A scFv comprises an amino acid
sequence:
(SEQ ID NO: 282, 1F3F3)
EVQLEESGGGLVQPGGSLKVSCAVSGLAFSSHDMSWVRQTPEKRLEWVAYISGGG
TNIYYSDTVKGRFTISRDNAKNTLYLQMSSLKSEDTAIYYCARPNYNYGGSWFAY
WGQGTLVTVSAGGGGSGGGGSGGGGSQIVLTQSPEIMSASPGEKVTMTCSASSSV
HYMHWYQQKSGTSPKRWIYDTSKLASGVPGRFSGSGSGTSYSLTISSMESEDAAT
YYCQQWTSNPPTFGGGTKLEIK.
In some embodiments, the anti-CLEC12A scFv comprises an amino acid
sequence:
(SEQ ID NO: 283)
QIVLTQSPEIMSASPGEKVTMTCSASSSVHYMHWYQQKSGTSPKRWIYDTSKLAS
GVPGRFSGSGSGTSYSLTISSMESEDAATYYCQQWTSNPPTFGGGTKLEIKGGGG
SGGGGSGGGGSELILVESGGGLVKPGGSLKLSCAVSGFTFSSFAMSWVRQTPEKR
LEWVATISSGGAYTFYKDSVKGRFTISRDNAKNTLYLQMSSLRSEDSAMYYCARH
SGYDGYYLYAMDYWGQGTSVTVSS.
In some embodiments, the anti-CLEC12A scFv comprises an amino acid
sequence:
(SEQ ID NO: 284)
QIVLTQSPEIMSASPGEKVTMTCSASSSVHYMHWYQQKSGTSPKRWIYDTSKLAS
GVPGRFSGSGSGTSYSLTISSMESEDAATYYCQQWTSNPPTFGGGTKLEIKGGGG
SGGGGSGGGGSGVQCELILVESGGGLVKPGGSLKLSCAVSGFTFSSFAVSWVRQT
PEKRLEWVATISSGGAYTFYKDSVKGRFTISRDNAKNTLYLQMSSLRSEDSAMYY
CARHSGYDGYYLYAMDYWGQGTSVTVSS.
In some embodiments, the anti-CLEC12A scFv comprises an amino acid
sequence:
(SEQ ID NO: 285)
QIVLTQSPEIMSASPGEKVTMTCSASSSVHYMHWYQQKSGTSPKRWIYDTSKLAS
GVPGRFSGSGSGTSYSLTISSMESEDAATYYCQQWTSNPPTFGGGTKLEIKGGGG
SGGGGSGGGGSEVQLEESGGGLVQPGGSLKVSCAVSGLAFSSHDMSWVRQTPEKR
LEWVAYISGGGTNIYYSDTVKGRFTISRDNAKNTLYLQMSSLKSEDTAIYYCARP
NYNYGGSWFAYWGQGTLVTVSA.

### CD83

For example, an scFv that selectively binds CD83 is described in US2020/0108098, which is incorporated by reference in its entirety for the description of these antibodies and their sequences.

For example, in some embodiments, the CDR1 sequence of the V_{H} domain comprises the amino acid sequence GFSITTGGYWWT (SEQ ID NO:286), SDGIS (SEQ ID NO:287), or SNAMI (SEQ ID NO:288); CDR2 sequence of the V_{H} domain comprises the amino acid sequence GYIFSSGNTNYNPSIKS (SEQ ID NO:289), IISSGGNTYYASWAKG (SEQ ID NO:290), orAMDSNSRTYYATWAKG (SEQ ID NO:291); CDR3 sequence of the V_{H} domain comprises the amino acid sequence CARAYGKLGFDY (SEQ ID NO:292), WGGTYSI (SEQ ID NO:293), or GDGGSSDYTEM (SEQ ID NO:294); CDR1 sequence of the V_{L} comprises the amino acid sequence TLSSQHSTYTIG (SEQ ID NO:295), QSSQSVYNNDFLS (SEQ ID NO:296), or QSSQSVYGNNELS (SEQ ID NO:297); CDR2 sequence of the V_{L} domain comprises the amino acid sequence VNSDGSHSKGD (SEQ ID NO:298), YASTLAS (SEQ ID NO:299), or QASSLAS (SEQ ID NO:300), and CDR3 sequence of the V_{L}domain comprises the amino acid sequence GSSDSSGYV (SEQ ID NO:301), TGTYGNSAWYEDA (SEQ ID NO:302), or LGEYSISADNH (SEQ ID NO:303).

For example, in some embodiments, the CDR1 sequence of the V_{H} domain comprises the amino acid sequence GFSITTGGYWWT (SEQ ID NO:304), CDR2 sequence of the V_{H} domain comprises the amino acid sequence GYIFSSGNTNYNPSIKS (SEQ ID NO:305), CDR3 sequence of the V_{H} domain comprises the amino acid sequence CARAYGKLGFDY (SEQ ID NO:306), CDR1 sequence of the V_{L} comprises the amino acid sequence TLSSQHSTYTIG (SEQ ID NO:307), CDR2 sequence of the V_{L} domain comprises the amino acid sequence VNSDGSHSKGD (SEQ ID NO:308), and CDR3 sequence of the V_{L} domain comprises the amino acid sequence GSSDSSGYV (SEQ ID NO:309).

For example, in some embodiments, the CDR1 sequence of the V_{H} domain comprises the amino acid sequence SDGIS (SEQ ID NO:310), CDR2 sequence of the V_{H} domain comprises the amino acid sequence IISSGGNTYYASWAKG (SEQ ID NO:311), CDR3 sequence of the V_{H} domain comprises the amino acid sequence WGGTYSI (SEQ ID NO:312), CDR1 sequence of the V_{L} comprises the amino acid sequence QSSQSVYNNDFLS (SEQ ID NO:313), CDR2 sequence of the V_{L} domain comprises the amino acid sequence YASTLAS (SEQ ID NO:314), and CDR3 sequence of the V_{L} domain comprises the amino acid sequence TGTYGNSAWYEDA (SEQ ID NO:315).

For example, in some embodiments, the CDR1 sequence of the V_{H} domain comprises the amino acid sequence SNAMI (SEQ ID NO:316), CDR2 sequence of the V_{H} domain comprises the amino acid sequence AMDSNSRTYYATWAKG (SEQ ID NO:317), CDR3 sequence of the V_{H} domain comprises the amino acid sequence GDGGSSDYTEM (SEQ ID NO:318), CDR1 sequence of the V_{L} comprises the amino acid sequence QSSQSVYGNNELS (SEQ ID NO:319), CDR2 sequence of the V_{L}domain comprises the amino acid sequence QASSLAS (SEQ ID NO:320), and CDR3 sequence of the V_{L} domain comprises the amino acid sequence LGEYSISADNH (SEQ ID NO:321).

In some embodiments, the anti-CD83 scFv V_{H} domain comprises the amino acid
sequence:
(SEQ ID NO: 322, VH-GBM00)
QVQLKESGPGLVKPSQSLSLTCSVTGFSITTGGYWWTWIRQFPGQKLEWMGYIFS
SGNTNYNPSIKSRISITRDTSKNQFFLQLNSVTTEGDTARYYCARAYGKLGFDYW
GQGTLVTVSS.
In some embodiments, the anti-CD83 scFv V_{L} domain comprises the amino acid
sequence:
(SEQ ID NO: 323, VL-GBM00)
QPVLTQSPSASASLGNSVKITCTLSSQHSTYTIGWYQQHPDKAPKYVMYVNSDGS
HSKGDGIPDRFSGSSSGAHRYLSISNIQPEDEADYFCGSSDSSGYVFGSGTQLTV
L.
In some embodiments, the anti-CD83 scFv V_{H} domain comprises the amino acid
sequence:
(SEQ ID NO: 324, 20D04)
METGLRWLLLVAVLKGVQCQSVEESGGRLVTPGTPLTLTCTVSGFSLSNNAINWV
RQAPGKGLEWIGYIWSGGLTYYANWAEGRFTISKTSTTVDLKMTSPTIEDTATYF
CARGINNSALWGPGTLVTVSSGQPKAPSVFPLAPCCGDTPSSTVTLGCLVKGYLP
EPVTVTWNSGTLTNGVRTFPSVRQSSGLYSLSSVVSVTSSSQPVTCNVAHPATNT
KVDKTVAPSTCSKPTCPPPELLGGPSVFIFPPKPKDTLMISRTPEVTCVVVDVSQ
DDPEVQFTWYINNEQVRTARPPLREQQFNSTIRVVSTLPIAHQDWLRGKEFKCKV
HNKALPAPIEKTISKARGQPLEPKVYTMGPPREELSSRSVSLTCMINGFYPSDIS
VEWEKNGKAEDNYKTTPAVLDSDGSYFLYNKLSVPTSEWQRGDVFTCSVMHEALH
NHYTQKSISRSPGK.
In some embodiments, the anti-CD83 scFv V_{L} domain comprises the amino acid
sequence:
(SEQ ID NO: 325, 20D04)
MDMRAPTQLLGLLLLWLPGARCADVVMTQTPASVSAAVGGTVTINCQASESISNY
LSWYQQKPGQPPKLLIYRTSTLASGVSSRFKGSGSGTEYTLTISGVQCDDVATYY
CQCTSGGKFISDGAAFGGGTEVVVKGDPVAPTVLLFPPSSDEVATGTVTIVCVAN
KYFPDVTVTWEVDGTTQTTGIENSKTPQNSADCTYNLSSTLTLTSTQYNSHKEYT
CKVTQGTTSVVQSFSRKNC.
In some embodiments, the anti-CD83 scFv V_{H} domain comprises the amino acid
sequence:
(SEQ ID NO: 326, 11G05)
METGLRWLLLVAVLKGVQCQSVEESGGRLVTPGTPLTLTCTVSGFTISDYDLSWV
RQAPGEGLKYIGFIAIDGNPYYATWAKGRFTISKTSTTVDLKITAPTTEDTATYF
CARGAGDLWGPGTLVTVSSGQPKAPSVFPLAPCCGDTPSSTVTLGCLVKGYLPEP
VTVTWNSGTLTNGVRTFPSVRQSSGLYSLSSVVSVTSSSQPVTCNVAHPATNTKV
DKTVAPSTCSKPTCPPPELLGGPSVFIFPPKPKDTLMISRTPEVTCVVVDVSQDD
PEVQFTWYINNEQVRTARPPLREQQFNSTIRVVSTLPIAHQDWLRGKEFKCKVHN
KALPAPIEKTISKARGQPLEPKVYTMGPPREELSSRSVSLTCMINGFYPSDISVE
WEKNGKAEDNYKTTPAVLDSDGSYFLYNKLSVPTSEWQRGDVFTCSVMHEALHNH
YTQKSISRSPGK.
In some embodiments, the anti-CD83 scFv V_{L} domain comprises the amino acid
sequence:
SEQ ID NO: 327, 11G05)
MDTREPTQLLGLLLLWLPGARCADVVMTQTPASVSAAVGGTVTINCQSSKNVYNN
NWLSWFQQKPGQPPKLLIYYASTLASGVPSRFRGSGSGTQFTLTISDVQCDDAAT
YYCAGDYSSSSDNGFGGGTEVVVKGDPVAPTVLLFPPSSDEVATGTVTIVCVANK
YFPDVTVTWEVDGTTQTTGIENSKTPQNSADCTYNLSSTLTLTSTQYNSHKEYTC
KVTQGTTSVVQSFSRKNC
In some embodiments, the anti-CD83 scFv V_{H} domain comprises the amino acid
sequence:
(SEQ ID NO: 328, 14C12)
METGLRWLLLVAVLKGVHCQSVEESGGRLVTPGTPLTLTCTASGFSRSSYDMSWV
RQAPGKGLEWVGVISTAYNSHYASWAKGRFTISRTSTTVDLKMTSLTTEDTATYF
CARGGSWLDLWGQGTLVTVSSGQPKAPSVFPLAPCCGDTPSSTVTLGCLVKGYLP
EPVTVTWNSGTLTNGVRTFPSVRQSSGLYSLSSVVSVTSSSQPVTCNVAHPATNT
KVDKTVAPSTCSKPTCPPPELLGGPSVFIFPPKPKDTLMISRTPEVTCVVVDVSQ
DDPEVQFTWYINNEQVRTARPPLREQQFNSTIRVVSTLPIAHQDWLRGKEFKCKV
HNKALPAPIEKTISKARGQPLEPKVYTMGPPREELSSRSVSLTCMINGFYPSDIS
VEWEKNGKAEDNYKTTPAVLDSDGSYFLYNKLSVPTSEWQRGDVFTCSVMHEALH
NHYTQKSISRSPGK.
In some embodiments, the anti-CD83 scFv V_{L} domain comprises the amino acid
sequence:
(SEQ ID NO: 329, 14C12)
MDXRAPTQLLGLLLLWLPGARCALVMTQTPASVSAAVGGTVTINCQSSQSVYDND
ELSWYQQKPGQPPKLLIYALASKLASGVPSRFKGSGSGTQFALTISGVQCDDAAT
YYCQATHYSSDWYLTFGGGTEVVVKGFPVAPTVLLFPPSSDEVATGTVTIVCVAN
KYFPDVTVTWEVDGTTQTTGTENSKTPQNSADCTYNLSSTLTLTSTQYNSHKEYT
CKVTQGTTSVVQSFSRKNC.
In some embodiments, the anti-CD83 scFv V_{H} domain comprises the amino acid
sequence:
(SEQ ID NO: 330, 020B08)
METGLRWLLLVAVLKGVQCQSVEESGGRLVTPGTPLTLTCTVSGFSLSSYDMTWV
RQAPGKGLEWIGIIYASGTTYYANWAKGRFTISKTSTTVDLKVTSPTIGDTATYF
CAREGAGVSMTLWGPGTLVTVSSGQPKAPSVFPLAPCCGDTPSSTVTLGCLVKGY
LPEPVTVTWNSGTLTNGVRTFPSVRQSSGLYSLSSVVSVTSSSQPVTCNVAHPAT
NTKVDKTVAPSTCSKPTCPPPELLGGPSVFIFPPKPKDTLMISRTPEVTCVVVDV
SQDDPEVQFTWYINNEQVRTARPPLREQQFNSTIRVVSTLPIAHQDWLRGKEFKC
KVHNKALPAPIEKTISKARGQPLEPKVYTMGPPREELSSRSVSLTCMINGFYPSD
ISVEWEKNGKAEDNYKTTPAVLDSDGSYFLYNKLSVPTSEWQRGDVFTCSVMHEA
LHNHYTQKSISRSPGK.
In some embodiments, the anti-CD83 scFv V_{L} domain comprises the amino acid
sequence:
(SEQ ID NO: 331, 020B08)
MDMRAPTQLLGLLLLWLPGARCAYDMTQTPASVEVAVGGTVTIKCQASQSISTYL
DWYQQKPGQPPKLLIYDASDLASGVPSRFKGSGSGTQFTLTISDLECADAATYYC
QQGYTHSNVDNVFGGGTEVVVKGDPVAPTVLLFPPSSDEVATGTVTIVCVANKYF
PDVTVTWEVDGTTQTTGIENSKTPQNSADCTYNLSSTLTLTSTQYNSHKEYTCKV
TQGTTSVVQSFSRKNC
In some embodiments, the anti-CD83 scFv V_{H} domain comprises the amino acid
sequence:
(SEQ ID NO: 332, 006G05)
METGLRWLLLVAVLKGVQCQSVEESGGRLVSPGTPLTLTCTASGFSLSSYDMSWV
RQAPGKGLEYIGIISSSGSTYYASWAKGRFTISKTSTTVDLEVTSLTTEDTATYF
CSREHAGYSGDTGHLWGPGTLVTVSSGQPKAPSVFPLAPCCGDTPSSTVTLGCLV
KGYLPEPVTVTWNSGTLTNGVRTFPSVRQSSGLYSLSSVVSVTSSSQPVTCNVAH
PATNTKVDKTVAPSTCSKPTCPPPELLGGPSVGIGPPKPKDTLMISRTPEVTCVV
VDVSQDDPEVQFTWYINNEQVRTARPPLREQQFNSTIRVVSTLPIAHQDWLRGKE
FKCKVHNKALPAPIEKTISKARGQPLEPKVYTMGPPREELSSRSVSLTCMINGFY
PSDISVEWEKNGKAEDNYKTTPAVLDSDGSYFLYNKLSVPTSEWQRGDVFTCSVM
HEALHNHYTQKSISRSPGK.
In some embodiments, the anti-CD83 scFv V_{L} domain comprises the amino acid
sequence:
(SEQ ID NO: 333, 006G05)
MDMRAPTQLLGLLLLWLPGARCAYDMTQTPASVEVAVGGTVAIKCQASQSVSSYL
AWYQQKPGQPPKPLIYEASMLAAGVSSRFKGSGSGTDFTLTISDLECDDAATYYC
QQGYSISDIDNAFGGGTEVVVKGDPVAPTVLLFPPSSDEVATGTVTIVCVANKYF
PDVTVTWEVDGTTQTTGIENSKTPQNSADCTYNLSSTLTLTSTQYNSHKEYTCKV
TQGTTSVVQSFSRKNC
In some embodiments, the anti-CD83 scFv V_{H} domain comprises the amino acid
sequence:
(SEQ ID NO: 334, 96G08)
METGLRWLLLVAVLKGVQCQSVEESGGRLVTPGTPLTLTCTVSGIDLSSDGISWV
RQAPGKGLEWIGIISSGGNTYYASWAKGRFTISRTSTTVDLKMTSLTTEDTATYF
CARVVGGTYSIWGQGTLVTVSSASTKGPSVYPLAPGSAAQTNSMVTLGCLVKGYF
PEPVTVTWNSGSLSSGVHTFPAVLQSDLYTLSSSVTVPSSTWPSETVTCNVAHPA
SSTKVDKKIVPRDCGCKPCICTVPEVSSVFIFPPKPDVLTITLTPKVTCVVVDIS
KDDPEVQFSWFVDDVEVHTAQTQPREEQFNSTFRSVSELPIMHQDWLNGKEFKCR
VNSAAFPAPIEKTISKTKGRPKAPQVYTIPPPKEQMAKDKVSLTCMITDFFPEDI
TVEWQWNGQPAENYKNTQPIMDTDGSYFVYSKLNVQKSNWEAGNTFTCSVLHEGL
HNHHTEKSLSHSPGK.
In some embodiments, the anti-CD83 scFv V_{L} domain comprises the amino acid
sequence:
(SEQ ID NO: 335, 96G08)
MDTRAPTQLLGLLLLWLPGATFAQVLTQTASPVSAPVGGTVTINCQSSQSVYNND
FLSWYQQKPGQPPKLLIYYASTLASGVPSRFKGSGSGTQFTLTISDLECDDAATY
YCTGTYGNSAWYEDAFGGGTEVVVKRTPVAPTVLLFPPSSAELATGTATIVCVAN
KYFPDGTVTWKVDGITQSSGINNSRTPQNSADCTYNLSSTLTLSSDEYNSHDEYT
CQVAQDSGSPVVQSFSRKSC
In some embodiments, the anti-CD83 scFv V_{H} domain comprises the amino acid
sequence:
(SEQ ID NO: 336, 95F04)
METGLRWLLLVAVLKGVQCQSVEESGGRLVTPGTPLTLTCTVSGIDLSSNAMIWV
RQAPREGLEWIGAMDSNSRTYYATWAKGRFTISRTSSITVDLKITSPTTEDTATY
FCARGDGGSSDYTEMWGPGTLVTVSSASTKGPSVYPLAPGSAAQTNSMVTLGCLV
KGYFPEPVTVTWNSGSLSSGVHTFPAVLQSDLYTLSSSVTVPSSTWPSETVTCNV
AHPASSTKVDKKIVPRDCGCKPCICTVPEVSSVFIFPPKPKDVLTITLTPKVTCV
VVDISKDDPEVQFSWFVDDVEVHTAQTQPREEQFNSTFRSVSELPIMHQDWLNGK
EFKCRVNSAAFPAPIEKTISKTKGRPKAPQVYTIPPPKEQMAKDKVSLTCMITDF
FPEDITVEWQWNGQPAENYKNTQPIMDTDGSYFVYSKLNVQKSNWEAGNTFTCSV
LHEGLHNHHTEKSLSHSPGK
In some embodiments, the anti-CD83 scFv V_{L} domain comprises the amino acid
sequence:
(SEQ ID NO: 337, 95F04)
MDTRAPTQLLGLLLLWLPGATFAQAVVTQTTSPVSAPVGGTVTINCQSSQSVYGN
NELSWYQQKPGQPPKLLIYQASSLASGVPSRFKGSGSGTQFTLTISDLECDDAAT
YYCLGEYSISADNHFGGGTEVVVKRTPVAPTVLLFPPSSAELATGTATIVCVANK
YFPDGTVTWKVDGITQSSGINNSRTPQNSADCTYNLSSTLTLSSDEYNSHDEYTC
QVAQDSGSPVVQSFSRKSC
In some embodiments, the anti-CD83 scFv V_{H} domain comprises the amino acid
sequence:
(SEQ ID NO: 338)
QVQLVQSGGAVVQPGRSLRLSCAASGFTFSTYGMHWVRQAPGKGLEWVAAVSYDG
SNKYYADFVKGRFTISRDNPKNTLYLQMNSLRADDTAVYYCARRGGLDIWGQGTT
VTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCAA
A.
In some embodiments, the anti-CD83 scFv V_{L} domain comprises the amino acid
sequence:
(SEQ ID NO: 339)
LTQPPPASGTPGQQRVTISCSGSSSNIGSNTVNWYQQLPGTAPKLLIYYGNDQRP
SGVPDRFSASKSGTSASLAISGLQSEDEAHYYCAAWDGSLNGGVIFGGGTKVTLG.
In some embodiments, the anti-CD83 scFv V_{L} domain comprises the amino acid
sequence:
(SEQ ID NO: 340)
VTQPPSASGTPGQRVTISCSGSSSNIGTNPVNWYQQLPGTAPKLLIYTTDQRPSG
VPDRFSGSKSGTSASLAISGLQSEDEADYYCAAWDDSLSGLYVFGTGTKVTVLG.
In some embodiments, the anti-CD83 scFv VL domain comprises the amino acid
sequence:
(SEQ ID NO: 341)
MTHTPLSLSVTPGQPASISCKSSQSLLHSDGKTYLYWYLQRPGQSPQPLIYEVSN
RFSGVPDRFSGSGSGTDFTLKISRVQAEDVGVYYCMQSLQLWTFGQGTKVEIKR.
In some embodiments, the anti-CD83 scFv V_{L} domain comprises the amino acid
sequence:
(SEQ ID NO: 342)
MTQSPLSLPVTLGQPASISCRSSQSLIHSDGNTYLDWFQQRPGQSPRRLIYKVSN
RDSGVPDRFSGSGSGTDFTLRISRVEAEDIGVYYCMQATHWPRTFGQGTKVEIKR.
In some embodiments, the anti-CD83 scFv V_{L} domain comprises the amino acid
sequence:
(SEQ ID NO: 343)
MTQSPLSLPVTLGQPASISCRSSQSLVDSAGNTFLHWFHQRPGQSPRRLIYKVSNR
DSGVPDRFSGSGSGTDFTLKISRVEAEDVGVYYCMQGTHWPRTFGQGTKVEIKR.
In some embodiments, the anti-CD83 scFv V_{L} domain comprises the amino acid
sequence:
(SEQ ID NO: 344)
LTQSPLSLPVTLGQPASISCKSSQSLVDSDGNTYLNWFQQRPGQSPRRLIYKVSN
RDSGVPDRFSGSGSGTDFTLKISRVEAEDVGVYYCMQGTHWPRTFGQGTKVEIKR.
In some embodiments, the anti-CD83 scFv V_{L} domain comprises the amino acid
sequence:
(SEQ ID NO: 345)
MTQSPLSLPVTLGQPASISCRSSQSLVHSDGNMYLNWFQQRPGQSPRRLIYKVSN
RDSGVPDRFSGSGSGTDFTLKISRVEAEDVGVYYCMQATQPTWTFGQGTKLEIKR.
In some embodiments, the anti-CD83 scFv V_{L} domain comprises the amino acid
sequence:
(SEQ ID NO: 346)
MTQSPSSLSASVGDRVTITCQASQDISNYLNWYQQKPGKAPKLLIYDASNLETGV
PSRFSGSGSGTDFTFTISSATYYCQQTYQGTKLEIKR.
In some embodiments, the anti-CD83 scFv V_{L} domain comprises the amino acid
sequence:
(SEQ ID NO: 347)
MTQSPSSLSASVGHPVTITCRASQSLISYLNWYHQKPGKAPKLLIYAASILQSGV
PSRFSGSGSGTDFTLTISSLQPENFASYYCQHTDSFPRTFGHGTKVEIKR.
In some embodiments, the anti-CD83 scFv V_{L} domain comprises the amino acid
sequence:
(SEQ ID NO: 348)
LTQPPSASGTPGQGVTISCRGSTSNIGNNVVNWYQHVPGSAPKLLIWSNIQRPSG
IPDRFSGSKSGTSASLAISGLQSEDQAVYYCAVWDDGLAGWVFGGGTTVTVLS.
In some embodiments, the anti-CD83 scFv V_{L} domain comprises the amino acid
sequence:
(SEQ ID NO: 349)
MTQAPVVSVALEQTVRITCQGDSLAIYYDFWYQHKPGQAPVLVIYGKNNRPSGIP
HRFSGSSSNTDSLTITGAQAEDEADYYCNSRDSSGNHWVFGGGTNLTVLG.
In some embodiments, the anti-CD83 scFv V_{L} domain comprises the amino acid
sequence:
(SSEQ ID NO: 350)
LTQSPLSLPVTLGQPASISCKSNQSLVHSDGNTYLNWFQQRPGQSPRRLIYKVSN
RDSGVPDRFSGSGSGTDFTLKINRVEAEDVGVYYCMQGTQWPRTFGGQGTKLDIK
R.
In some embodiments, the anti-CD83 scFv V_{H} domain has been humanized and
comprises the amino acid sequence:
(SEQ ID NO: 351, VH-GBM01)
QVQLQESGPGLVKPSETLSLTCTVSGFSITTGGYWWTWIRQPPGKGLEWIGYIFS
SGNTNYNPSIKSRVTISVDTSKNQFSLKLSSVTAADTAVYYCARAYGKLGFDYWG
QGTLVTVSS.
In some embodiments, the anti-CD83 scFv V_{H} domain has been humanized and
comprises the amino acid sequence:
(SEQ ID NO: 352, VH-GBM02)
QVQLQESGPGLVKPSQTLSLTCTVSGFSITTGGYWWTWIRQHPGKGLEWIGYIFS
SGNTNYNPSIKSLVTISVDTSKNQFSLKLSSVTAADTAVYYCARAYGKLGFDYWG
QGTLVTVSS.
In some embodiments, the anti-CD83 scFv V_{H} domain has been humanized and
comprises the amino acid sequence:
(SEQ ID NO: 353, VH-GBM03)
QVQLQESGPGLVKPSQTLSLTCTVSGFSITTGGYWWTWIRQPPGKGLEWIGYIFS
SGNTNYNPSIKSRVTISVDTSKNQFSLKLSSVTAADTAVYYCARAYGKLGFDYWG
QGTLVTVSS.
In some embodiments, the anti-CD83 scFv V_{H} domain has been humanized and
comprises the amino acid sequence:
(SEQ ID NO: 354, VH-GBM04)
QVQLQESGPGLVKPSETLSLTCTVSGFSITTGGYWWTWIRQPPGKGLEWIGYIFS
SGNTNYNPSIKSRVTISRDTSKNQFSLKLSSVTAADTAVYYCARAYGKLGFDYWG
QGTLVTVSS.
In some embodiments, the anti-CD83 scFv V_{H} domain has been humanized and
comprises the amino acid sequence:
(SEQ ID NO: 355, VH-GBM05)
QVQLQESGPGLVKPSETLSLTCTVSGFSITTGGYWWTWIRQPPGKGLEWIGYIFS
SGNTNYNPSIKSRVTISVDTSKNQFSLKLSSVTAADTARYYCARAYGKLGFDYWG
QGTLVTVSS.
In some embodiments, the anti-CD83 scFv V_{H} domain has been humanized and
comprises the amino acid sequence:
(SEQ ID NO: 356, VH-GBM06)
QVQLQESGPGLVKPSETLSLTCTVSGFSITTGGYWWTWIRQPPGKGLEWIGYIFS
SGNTNYNPSIKSRISITRDTSKNQFFLQLNSVTTEGDTARYYCARAYGKLGFDYW
GQGTLVTVSS.
In some embodiments, the anti-CD83 scFv V_{L} domain has been humanized and
comprises the amino acid sequence:
(SEQ ID NO: 357, VL-GBM01)
QLVLTQSPSASASLGASVKLTCTLSSQHSTYTIGWHQQQPEKGPRYLMKVNSDGS
HSKGDGIPDRFSGSSSGAERYLTISSLQSEDEADYYCGSSDSSGYVFGSGTKVTV
L.
In some embodiments, the anti-CD83 scFv V_{L} domain has been humanized and
comprises the amino acid sequence:
(SEQ ID NO: 358, VL-GBM02)
LPVLTQPPSASALLGASIKLTCTLSSQHSTYTIGWYQQRPGRSPQYIMKVNSDGS
HSKGDGIPDRFMGSSSGADRYLTFSNLQSDDEAEYHCGSSDSSGYVFGSGTKVTV
L.
In some embodiments, the anti-CD83 scFv comprises an amino acid sequence:
(SEQ ID NO: 359)
QPVLTQSPSASASLGNSVKITCTLSSQHSTYTIGWYQQHPDKAPKYVMYVNSDGS
HSKGDGIPDRFSGSSSGAHRYLSISNIQPEDEADYFCGSSDSSGYVFGSGTQLTV
LRAAASSGGGGSGGGGSGGGGSQPVLTQSPSASASLGNSVKITCTLSSQHSTYTI
GWYQQHPDKAPKYVMYVNSDGSHSKGDGIPDRFSGSSSGAHRYLSISNIQPEDEA
DYFCGSSDSSGYVFGSGTQLTVLRAAA.
In some embodiments, the anti-CD83 scFv comprises an amino acid sequence:
(SEQ ID NO: 360)
QVQLKESGPGLVKPSQSLSLTCSVTGFSITTGGYWWTWIRQFPGQKLEWMGYIFS
SGNTNYNPSIKSRISITRDTSKNQFFLQLNSVTTEGDTARYYCARAYGKLGFDYW
GQGTLVTVSSGGGGSGGGGSGGGGSQVQLKESGPGLVKPSQSLSLTCSVTGFSIT
TGGYWWTWIRQFPGQKLEWMGYIFSSGNTNYNPSIKSRISITRDTSKNQFFLQLN
SVTTEGDTARYYCARAYGKLGFDYWGQGTLVTV.
In some embodiments, the anti-CD83 scFv comprises an amino acid sequence:
(SEQ ID NO: 361)
QVQLQESGPGLVKPSETLSLTCTVSGFSITTGGYWWTWIRQPPGKGLEWIGYIFS
SGNTNYNPSIKSRVTISVDTSKNQFSLKLSSVTAADTAVYYCARAYGKLGFDYWG
QGTLVTVSSGGGGSGGGGSGGGGSQLVLTQSPSASASLGASVKLTCTLSSQHSTY
TIGWHQQQPEKGPRYLMKVNSDGSHSKGDGIPDRFSGSSSGAERYLTISSLQSED
EADYYCGSSDSSGYVFGSGTKVTVL.
In some embodiments, the anti-CD83 scFv comprises an amino acid sequence:
(SEQ ID NO: 362)
QVQLQESGPGLVKPSQTLSLTCTVSGFSITTGGYWWTWIRQHPGKGLEWIGYIFS
SGNTNYNPSIKSLVTISVDTSKNQFSLKLSSVTAADTAVYYCARAYGKLGFDYWG
QGTLVTVSSGGGGSGGGGSGGGGSQLVLTQSPSASASLGASVKLTCTLSSQHSTY
TIGWHQQQPEKGPRYLMKVNSDGSHSKGDGIPDRFSGSSSGAERYLTISSLQSED
EADYYCGSSDSSGYVFGSGTKVTVL.
In some embodiments, the anti-CD83 scFv comprises an amino acid sequence:
(SEQ ID NO: 363)
QVQLQESGPGLVKPSQTLSLTCTVSGFSITTGGYWWTWIRQPPGKGLEWIGYIFS
SGNTNYNPSIKSRVTISVDTSKNQFSLKLSSVTAADTAVYYCARAYGKLGFDYWG
QGTLVTVSSGGGGSGGGGSGGGGSQLVLTQSPSASASLGASVKLTCTLSSQHSTY
TIGWHQQQPEKGPRYLMKVNSDGSHSKGDGIPDRFSGSSSGAERYLTISSLQSED
EADYYCGSSDSSGYVFGSGTKVTVL.
In some embodiments, the anti-CD83 scFv comprises an amino acid sequence:
QVQLQESGPGLVKPSETLSLTCTVSGFSITTGGYWWTWIRQPPGKGLEWIGYIFSSGN
(SEQ ID NO: 364)
TNYNPSIKSRVTISRDTSKNQFSLKLSSVTAADTAVYYCARAYGKLGFDYWGQGT
LVTVSSGGGGSGGGGSGGGGSQLVLTQSPSASASLGASVKLTCTLSSQHSTYTIG
WHQQQPEKGPRYLMKVNSDGSHSKGDGIPDRFSGSSSGAERYLTISSLQSEDEAD
YYCGSSDSSGYVFGSGTKVTVL.
In some embodiments, the anti-CD83 scFv comprises an amino acid sequence:
(SEQ ID NO: 365)
QVQLQESGPGLVKPSETLSLTCTVSGFSITTGGYWWTWIRQPPGKGLEWIGYIFS
SGNTNYNPSIKSRVTISVDTSKNQFSLKLSSVTAADTARYYCARAYGKLGFDYWG
QGTLVTVSSGGGGSGGGGSGGGGSQLVLTQSPSASASLGASVKLTCTLSSQHSTY
TIGWHQQQPEKGPRYLMKVNSDGSHSKGDGIPDRFSGSSSGAERYLTISSLQSED
EADYYCGSSDSSGYVFGSGTKVTVL.
In some embodiments, the anti-CD83 scFv comprises an amino acid sequence:
(SEQ ID NO: 366)
QVQLQESGPGLVKPSETLSLTCTVSGFSITTGGYWWTWIRQPPGKGLEWIGYIFS
SGNTNYNPSIKSRISITRDTSKNQFFLQLNSVTTEGDTARYYCARAYGKLGFDYW
GQGTLVTVSSGGGGSGGGGSGGGGSQLVLTQSPSASASLGASVKLTCTLSSQHST
YTIGWHQQQPEKGPRYLMKVNSDGSHSKGDGIPDRFSGSSSGAERYLTISSLQSE
DEADYYCGSSDSSGYVFGSGTKVTVL.
In some embodiments, the anti-CD83 scFv comprises an amino acid sequence:
(SEQ ID NO: 367)
QVQLQESGPGLVKPSETLSLTCTVSGFSITTGGYWWTWIRQPPGKGLEWIGYIFS
SGNTNYNPSIKSRVTISVDTSKNQFSLKLSSVTAADTAVYYCARAYGKLGFDYWG
QGTLVTVSSGGGGSGGGGSGGGGSLPVLTQPPSASALLGASIKLTCTLSSQHSTY
TIGWYQQRPGRSPQYIMKVNSDGSHSKGDGIPDRFMGSSSGADRYLTFSNLQSDD
EAEYHCGSSDSSGYVFGSGTKVTVL.
In some embodiments, the anti-CD83 scFv comprises an amino acid sequence:
(SEQ ID NO: 368)
QVQLQESGPGLVKPSQTLSLTCTVSGFSITTGGYWWTWIRQHPGKGLEWIGYIFS
SGNTNYNPSIKSLVTISVDTSKNQFSLKLSSVTAADTAVYYCARAYGKLGFDYWG
QGTLVTVSSGGGGSGGGGSGGGGSLPVLTQPPSASALLGASIKLTCTLSSQHSTY
TIGWYQQRPGRSPQYIMKVNSDGSHSKGDGIPDRFMGSSSGADRYLTFSNLQSDD
EAEYHCGSSDSSGYVFGSGTKVTVL.
In some embodiments, the anti-CD83 scFv comprises an amino acid sequence:
(SEQ ID NO: 369)
QVQLQESGPGLVKPSQTLSLTCTVSGFSITTGGYWWTWIRQPPGKGLEWIGYIFS
SGNTNYNPSIKSRVTISVDTSKNQFSLKLSSVTAADTAVYYCARAYGKLGFDYWG
QGTLVTVSSGGGGSGGGGSGGGGSLPVLTQPPSASALLGASIKLTCTLSSQHSTY
TIGWYQQRPGRSPQYIMKVNSDGSHSKGDGIPDRFMGSSSGADRYLTFSNLQSDD
EAEYHCGSSDSSGYVFGSGTKVTVL.
In some embodiments, the anti-CD83 scFv comprises an amino acid sequence:
(SEQ ID NO: 370)
QVQLQESGPGLVKPSETLSLTCTVSGFSITTGGYWWTWIRQPPGKGLEWIGYIFS
SGNTNYNPSIKSRVTISRDTSKNQFSLKLSSVTAADTAVYYCARAYGKLGFDYWG
QGTLVTVSSGGGGSGGGGSGGGGSLPVLTQPPSASALLGASIKLTCTLSSQHSTY
TIGWYQQRPGRSPQYIMKVNSDGSHSKGDGIPDRFMGSSSGADRYLTFSNLQSDD
EAEYHCGSSDSSGYVFGSGTKVTVL.
In some embodiments, the anti-CD83 scFv comprises an amino acid sequence:
(SEQ ID NO: 371)
QVQLQESGPGLVKPSETLSLTCTVSGFSITTGGYWWTWIRQPPGKGLEWIGYIFS
SGNTNYNPSIKSRVTISVDTSKNQFSLKLSSVTAADTARYYCARAYGKLGFDYWG
QGTLVTVSSGGGGSGGGGSGGGGSLPVLTQPPSASALLGASIKLTCTLSSQHSTY
TIGWYQQRPGRSPQYIMKVNSDGSHSKGDGIPDRFMGSSSGADRYLTFSNLQSDD
EAEYHCGSSDSSGYVFGSGTKVTVL.
In some embodiments, the anti-CD83 scFv comprises an amino acid sequence:
(SEQ ID NO: 372)
QVQLQESGPGLVKPSETLSLTCTVSGFSITTGGYWWTWIRQPPGKGLEWIGYIFS
SGNTNYNPSIKSRISITRDTSKNQFFLQLNSVTTEGDTARYYCARAYGKLGFDYW
GQGTLVTVSSGGGGSGGGGSGGGGSLPVLTQPPSASALLGASIKLTCTLSSQHST
YTIGWYQQRPGRSPQYIMKVNSDGSHSKGDGIPDRFMGSSSGADRYLTFSNLQSD
DEAEYHCGSSDSSGYVFGSGTKVTVL.
In some embodiments, the anti-CD83 scFv comprises an amino acid sequence:
(SEQ ID NO: 373)
QVQLKESGPGLVKPSQSLSLTCSVTGFSITTGGYWWTWIRQFPGQKLEWMGYIFS
SGNTNYNPSIKSRISITRDTSKNQFFLQLNSVTTEGDTARYYCARAYGKLGFDYW
GQGTLVTVSSGGGGSGGGGSGGGGSQPVLTQSPSASASLGNSVKITCTLSSQHST
YTIGWYQQHPDKAPKYVMYVNSDGSHSKGDGIPDRFSGSSSGAHRYLSISNIQPE
DEADYFCGSSDSSGYVFGSGTQLTVL.

### PSCA

For example, an scFv that selectively binds PSCA is described in US2021/0379108, which is incorporated by reference in its entirety for the description of these antibodies and their sequences. For example, in some embodiments, the anti-PSCA scFv has the amino acid sequence:

(SEQ ID NO: 374)
MVLLVTSLLLCELPHPAFLLIPQVQLQESGPGLVKPSQTLSLTCT
VSGGSISSGGYYWIWIRQHPGKGLEWIGYIYYNGNTYYNPSLKSR
VTMSVDTSKNQFSLKLSSVTAADTAVYYCARDGITMIRGYYYGMD
VWGQGTTVTVSSGGGGSGGGGSGGGGSDIQLTQSPSSVSASVGDR
VTITCRASRGISSWLAWYQQKPGKAPKLLIYTASSLQSGVPSRFS
GSGSGTDFTLTISSLOPEDFATYYCQQAYSFPRTFGQGTKVEIKA
AAFV.

### OR2H1

For example, an scFv that selectively binds OR2H1 is described in WO2021257905, which is incorporated by reference in its entirety for the description of these antibodies and their sequences.

In some embodiments of the anti-OR2H1 scFv, the CDR1 sequence of the V_{H} domain comprises the amino acid sequence GYIFTGYY (SEQ ID NO:375); CDR2 sequence of the V_{H} domain comprises the amino acid sequence NAKSGE (SEQ ID NO:376); CDR3 sequence of the V_{H} domain comprises the amino acid sequence GPLL (SEQ ID NO:377); CDR1 sequence of the V_{L} comprises the amino acid sequence SSGSSNIGSNFVS (SEQ ID NO:378); CDR2 sequence of the V_{L} domain comprises the amino acid sequence RNNQRPS (SEQ ID NO:379); and CDR3 sequence of the V_{L}domain comprises the amino acid sequence AAWDDSVRGPV (SEQ ID NO:380). In some embodiments, the anti-OR2H1 scFv V_{H} domain has been humanized and comprises the amino acid sequence: QVQLQQSGAEVKKPGESLKISCKGSGYIFTGYYMHWVRQAPGQRPEWLGRMNAKSG EADSAQRFQGRVTMTRDTSINTAYMELRDLRSDDTAVYYCTRGPLLWGQGTLVTVS (SEQ ID NO:381). In some embodiments, the anti-OR2H1 V_{L} domain has been humanized and comprises the amino acid sequence:

(SEQ ID NO: 382)
QPVLTQPPSASGTPGQRVTISCSGSSSNIGSNFVSWYQQLPGTAP
KLLIYRNNQRPSGVPDRFSGSKTGTSASLAISGLRSEDEADYYCA
AWDDSVRGPVFGGGTELTVLAAA.

In some embodiments of the anti-OR2H1 scFv, the CDR1 sequence of the V_{H} domain comprises the amino acid sequence GYIFTGYY (SEQ ID NO:383); CDR2 sequence of the V_{H} domain comprises the amino acid sequence MNAKSGEA (SEQ ID NO:384); CDR3 sequence of the V_{H} domain comprises the amino acid sequence TRGPLL (SEQ ID NO:385); CDR1 sequence of the V_{L} comprises the amino acid sequence SSNIGSNF (SEQ ID NO:386); CDR2 sequence of the V_{L} domain comprises the amino acid sequence RNN; and CDR3 sequence of the V_{L} domain comprises the amino acid sequence AAWDDSVRGPV (SEQ ID NO:74). In some embodiments, the anti-OR2H1 scFv V_{H} domain has been humanized and comprises the amino acid sequence: MAQVQLQQSGAEVKKPGESLKISCKGSGYIFTGYYMHWVRQAPGQRPEWLGRMNAK SGEADSAQRFQGRVTMTRDTSINTAYMELRDLRSDDTAVYYCTRGPLLWGQGTLVTV SS (SEQ ID NO:75). In some embodiments, the anti-OR2H1 V_{L} domain has been humanized and comprises the amino acid sequence:

(SEQ ID NO: 75)
QPVLTQPPSASGTPGQRVTISCSGSSSNIGSNFVSWYQQLPGTAP
KLLIYRNNQRPSGVPDRFSGSKTGTSASLAISGLRSEDEADYYCA
AWDDSVRGPVFGGGTELTVL.

In some embodiments of the anti-OR2H1 scFv, the CDR1 sequence of the V_{H} domain comprises the amino acid sequence SSNIGSNF (SEQ ID NO:77); CDR2 sequence of the V_{H} domain comprises the amino acid sequence RNN; CDR3 sequence of the V_{H} domain comprises the amino acid sequence AAWDDSVRGPV (SEQ ID NO:125); CDR1 sequence of the V_{L} comprises the amino acid sequence GYTFTSNY (SEQ ID NO:139); CDR2 sequence of the V_{L} domain comprises the amino acid sequence INPSGGRT (SEQ ID NO:176); and CDR3 sequence of the V_{L} domain comprises the amino acid sequence ARSHCSGGSCYSIDY (SEQ ID NO:269). In some embodiments, the anti-OR2H1 scFv V_{H} domain has been humanized and comprises the amino acid sequence: EVQLVQSGAEVKKPGASVKVSCKASGYTFTSNYMHWVRQAPGQGLEWMGIINPSGG RTSYAQKFQGRVTMTRDTSTGTVYMELSSLRSEDTAVYYCARSHCSGGSCYSIDYWG QGTLVTVSS (SEQ ID NO:387). In some embodiments, the anti-OR2H1 V_{L} domain has been humanized and comprises the amino acid sequence:

(SEQ ID NO: 124)
QPVLTQSSSASASLGSSVKLTCTLSSGHSGYIIAWHQQQPGKAPR
YLMKVEGSGSYNKGSGIPERFSGSSSGADRYLTISNLQSEDEADY
YCETWDSNTHVFGTGTKVTVL.

Additional scFv sequences are disclosed in U.S. Pat. Nos. 7,575,742, 8,298,525, 8,636,997, 9,394,368, 9,447,194, 9,624,306, 9,765,342, 10,767,184, 10,815,487, 10,738,312, 10,738,313, 10,844,387, 10,829,767, 10,900,042, 10,876,123, 10,815,488, 10,829,768, 10,837,019, and 10,829,769, US2013/0071414, US2014/0023647, US2014/0271635, US2014/0286987, US2015/0283178, US2015/0344844, US2016/0046724, US2016/0046700, US2016/0152723, US2016,0297884, US2016,0303230, US2016,0376375, US2017,0183418, US2017,0218337, US2017,0226216, US2017/0283504, US2017/0368101, WO2016/130598, and WO2019/195017 which are incorporated by reference in their entireties for the teaching of these scFv sequences.

In some embodiments, the scFv is the anti-BCMA02 scFv (ABECMA; idecabtagene vicleucel) disclose din 9,765,342. In some embodiments, the scFv is the anti-CD19 scFv (BREYANZI; lisocabtagene maraleucel) disclosed in 9,701,758. In some embodiments, the scFv is the anti-CD19 scFv (KYMRIAH; tisagenlecleucel) disclosed in US2014/0271635. In some embodiments, the scFv is the anti-CD19 scFv scFv (TECARTUS; brexucabtagene autoleucel) disclosed in US2015/0344844. In some embodiments, the scFv is the anti-CD19 scFv (YESCARTA; axicabtagene ciloleucel) disclosed in US2015/0344844.

Also disclosed are isolated nucleic acid sequences encoding the disclosed CAR polypeptides, vectors comprising these isolated nucleic acids, and cells containing these vectors. For example, the cell can be an immune effector cell selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell.

In some embodiments, the cell exhibits an anti-tumor immunity when the antigen binding domain of the CAR binds to a TAA on a tumor.

Also disclosed is a method of providing an anti-tumor immunity in a subject with a TAA-expressing cancer that involves administering to the subject an effective amount of an immune effector cell genetically modified with a disclosed TAA-specific CAR.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show Hela (FIG. 1A) and BT-549 (FIG. 1B) cell killing (%) for OR5V1 and OR5V1-NKG2D CARs.

FIG. 2 shows Hela tumor volume after treatment with OR5V1 or OR5V1-NKG2D CARs.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “protein domain” refers to a portion of a protein, portions of a protein, or an entire protein showing structural integrity; this determination may be based on amino acid composition of a portion of a protein, portions of a protein, or the entire protein.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid subsitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

Chimeric Antigen Receptors (CAR) with NKG2D Intracellular Domain

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein is a chimeric antigen receptor (CAR) that can be that can be expressed in immune effector cells to enhance antitumor activity against cancers.

The disclosed CAR is generally made up of three domains: an ectodomain, a transmembrane domain, and an endodomain. The ectodomain comprises the binding region and is responsible for antigen recognition. It also optionally contains a signal peptide (SP) so that the CAR can be glycosylated and anchored in the cell membrane of the immune effector cell. The transmembrane (TM) domain, is as its name suggests, connects the ectodomain to the endodomain and resides within the cell membrane when expressed by a cell. The endodomain is the business end of the CAR that transmits an activation signal to the immune effector cell after antigen recognition. The disclosed CARs contain in the endodomain the intracellular domain of NKG2D.

In some embodiments, the disclosed CAR is defined by the formula:

SP-BD-HG-TM-NKRIC;

wherein “SP” represents an optional signal peptide,
wherein “BD” represents a target binding domain,
wherein “HG” represents an optional hinge domain,
wherein “TM” represents a transmembrane domain,
wherein “NKRIC” represents an NK cell receptor intracellular domain, and
wherein “-” represents a peptide bond or linker.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The binding domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. The disclosed CARs contain in the endodomain the intracellular domain of NKG2D. However, in some embodiments, the endodomain may further comprise the “intracellular signaling domain” of a T cell receptor (TCR) and optional co-receptors. For example, the endodomain of the CAR can be designed to further comprise the CD3ζ signaling domain by itself or combined with any other desired cytoplasmic domain(s) useful in the context of the CAR. For example, the cytoplasmic domain of the CAR can further comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1 BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety and the transmembrane domain. The hinge sequence can be any suitable sequence derived or obtained from any suitable molecule. In some embodiments, for example, the hinge sequence is derived from a CD8a molecule or a CD28 molecule. In some embodiments, the hinge sequence is derived from an NK receptor, preferably the same NK receptor from which the intracellular domain is derived.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction.

In some embodiments, the binding domain is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some cases, the binding domain is an affinity maturated scFv. In some cases, the binding domain has a dissociation constant (K_{D}) for the TAA that is less than 50 nM, 40 nM, 30 nM, 25 nM, 20 nM, 15 nM, or 10 nM.

In some embodiments, the binding domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Tumor antigens are proteins that are produced by tumor cells that elicit an immune response, particularly T-cell mediated immune responses. The binding domain can be an antibody or a natural ligand of the tumor antigen. The selection of the antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, CD33, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, NB/70K, NY-CO-1, RCASI, SDCCAG1 6, TA-90Mac-2 binding proteincyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed CARs that allow expression of the CARs in the disclosed immune effector cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodimens, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells

Also disclosed are immune effector cells that are engineered to express the disclosed CARs (also referred to herein as “CAR-T cells”). These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8+ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-1-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

### Therapeutic Methods

Immune effector cells expressing the disclosed CARs can elicit an anti-tumor immune response against TAA-expressing cancer cells. The anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to TAA.

Adoptive transfer of immune effector cells expressing chimeric antigen receptors is a promising anti-cancer therapeutic. Following the collection of a patient's immune effector cells, the cells may be genetically engineered to express the disclosed CARs, then infused back into the patient.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodimetns formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any TAA-expressing cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed CARs can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CARs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec ST1571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a CARs for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with an CARs for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CARs is administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CARs is administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

A tandem CAR contains two sequential antigen binding domains facing the extracellular environment connected to the intracellular costimulatory and stimulatory domains.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC tag, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional-CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

NKG2D is a transmembrane stimulatory receptor expressed on the surfaces of natural killer (NK) cells, NKT cells, human CD8 T cells, and mouse CD8 T cells (Raulet DH. Nat Rev Immunol 2003 3:781-90). In CD8 T cells, NKG2D is expressed in association with its adaptor molecule DAP10 (Wu J, et al. Science 1999 285:730-2). The ligands for NKG2D receptors are ULBP1-4, MICA, MICB in humans, and Rae1, MULT1, and H60 in mouse (Garrity D, et al. Proc Natl Acad Sci 2005 102:7641-6). In CD8 T cells, ligation of NKG2D receptor to its ligands enhances the activation, proliferation, and production of inflammatory cytokines and effector molecules 1. NKG2D can mediate the survival of memory CD8 T cells and is sufficient to maintain the memory formation in CD8 T cells in the absence of CD4 T cell ‘help’ (Zloza A, et al. Nat Med 2012 18:422-8).

In CD8 T cells, DAP10 expresses the YxNM domain, which recruits p85 to induce PI3K signaling, and the YxNX domain is associated with Grb2 recruitment and signaling (Okkenhaug K, et al. Nat Rev Immunol 2003 3:317-30; Upshaw J L, et al. Nat Immunol 2006 7:524-32).

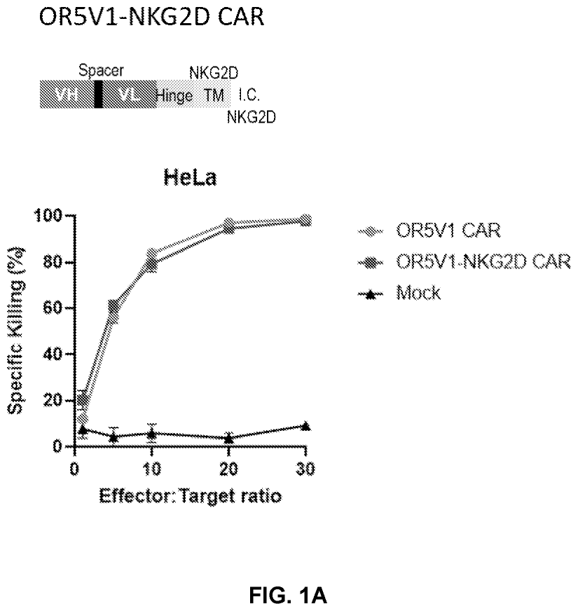

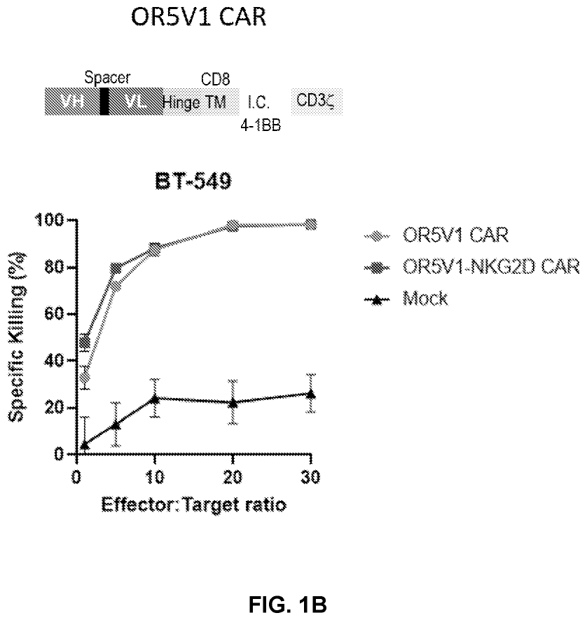

The goal of this study was to engineer scFv CAR T cells with Pro-T cell properties (killing, survival, and migratory properties) by utilizing the NKG2D/DAP10 signaling pathways. To achieve this goal, an scFv against OR5V1 was linked to the transmembrane domain (Tm) and the intracellular domain (IC) of NKG2D (FIG. 1A). In this design, the scFv will recognize the antigen resulting in the activation of the NKG2D/DAP10 signaling that in turn will initiate the killing and memory phase in the carrying T cells.

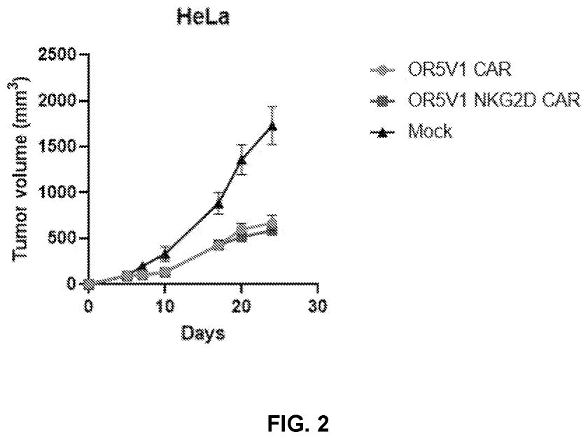

The designed construct was expressed in human T cells and tested side by side with 2nd generation CAR-T cell for their ability to recognize tumor cells expressing the scFv target. The capacity of ORV1-NKG2D-CAR to kill target cells (HeLa, cervical cancer) and BT-549 cells (papillary, invasive ductal tumor) was indistinguishable from regular CAR-T cells (FIGS. 2A and 2B). These data show that the NKG2D (TM-IC) can substitute the regular CAR-T cell signaling that utilizes CD3 domains.

Next, it was determined if ORV1-NKG2D-CAR can kill tumors in vivo compared to regular CAR-T cells. FIG. 3 shows that ORV1-NKG2D-CAR controls tumor growth similar to the regular CAR-T. In this experiment, immune-deficient mice (NSG mice) were tumor-challenged with HeLa cells in the flank and treated with mock engineered T cells, regular CAR-T, and CAR-T NKG2D. Tumor growth was manually measured and plotted. These data show that the scFv-NKG2D (TM-IC) can substitute the regular CAR-T cell signaling that utilizes CD3 domains and mediate in vivo anti-tumor responses against a human tumor.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A chimeric antigen receptor (CAR) polypeptide, comprising a ligand binding domain, a hinge domain, a transmembrane domain, and an endodomain that comprises an NK cell receptor intracellular domain, or fragment thereof capable of activating the target killing pathway.

2. The polypeptide of claim 1, wherein the NK cell receptor is hNKG2D, hCD16, hNKp30, hNKG2C, h2B4, hDNAM-1, hCD137, hOX-40, hCD27, KIR2DS5, KIR3DS1, or NKp80/KLRF1.

3. The polypeptide of claim 2, wherein the intracellular domain comprises the amino acid sequence SEQ ID NO:1, SEQ ID NO:4, SEQ ID NO:7, SEQ ID NO:10, SEQ ID NO:13, SEQ ID NO:16, SEQ ID NO:19, SEQ ID NO:22, SEQ ID NO:25, SEQ ID NO:28, SEQ ID NO:31, or SEQ ID NO:34.

4. The polypeptide of claim 3, wherein the transmembrane domain comprises the amino acid sequence SEQ ID NO:2, SEQ ID NO:5, SEQ ID NO:8, SEQ ID NO:11, SEQ ID NO:14, SEQ ID NO:17, SEQ ID NO:20, SEQ ID NO:23, SEQ ID NO:26, SEQ ID NO:29, SEQ ID NO:32, or SEQ ID NO:35.

5. The polypeptide of claim 2, comprising the amino acid sequence SEQ ID NO:3, SEQ ID NO:6, SEQ ID NO:9, SEQ ID NO:12, SEQ ID NO:15, SEQ ID NO:18, SEQ ID NO:21, SEQ ID NO:24, SEQ ID NO:27, SEQ ID NO:30, SEQ ID NO:33, or SEQ ID NO:36.

6. The polypeptide of any one of claims 1 to 5, wherein the endodomain does not contain a CD3 zeta (CD3ζ) signaling domain.

7. The polypeptide of claim 1, wherein the CAR polypeptide is defined by the formula:
SP-BD-HG-TM-NKRIC;
wherein “SP” represents an optional signal peptide,
wherein “BD” represents a target binding domain,
wherein “HG” represents an optional hinge domain,
wherein “TM” represents a transmembrane domain,
wherein “NKRIC” represents an NK cell receptor intracellular domain, and
wherein “-” represents a peptide bond or linker.

8. An isolated nucleic acid sequence encoding the recombinant polypeptide of claim 1.

9. A vector comprising the isolated nucleic acid sequence of claim 8.

10. A cell comprising the vector of claim 9.

11. The cell of claim 10, wherein the cell is selected from the group consisting of an αβT cell, γδT cell, a Natural Killer (NK) cells, a Natural Killer T (NKT) cell, an innate lymphoid cell (ILC), a cytokine induced killer (ClK) cell, a cytotoxic T lymphocyte (CTL), a lymphokine activated killer (LAK) cell, a regulatory T (T_{reg}) cell, or any combination thereof.

12. The cell of claim 11, wherein the cell exhibits an anti-tumor immunity when the antigen binding domain of the CAR binds to TAA.

13. A method of providing an anti-tumor immunity in a subject with a TAA-expressing cancer, the method comprising administering to the subject an effective amount of an immune effector cell genetically modified to express the CAR polypeptide of claim 1, thereby providing an anti-tumor immunity in the mammal.

14. The method of claim 13, wherein the immune effector cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T (T_{reg}) cell, or any combination thereof.

15. The method of claim 13, further comprising administering to the subject a checkpoint inhibitor.

16. The method of claim 15, wherein the checkpoint inhibitor comprises an anti-PD-1 antibody, anti-PD-L1 antibody, anti-CTLA-4 antibody, or a combination thereof.


SEQUENCE LISTING (387 sequences)

SEQ ID NO: 1 - protein, 51 aa, synthetic construct
MGWIRGRRSRHSWEMSEFHNYNLDLKKSDFSTRWQKQRCPVVKSKCRENAS

SEQ ID NO: 2 - protein, 23 aa, synthetic construct
PFFFCCFIAVAMGIRFIIMVTIW

SEQ ID NO: 3 - protein, 74 aa, synthetic construct
PFFFCCFIAVAMGIRFIIMVTIWMGWIRGRRSRHSWEMSEFHNYNLDLKKSDFSTRWQKQRCPVVKSKCRENAS

SEQ ID NO: 4 - protein, 25 aa, synthetic construct
KTNIRSSTRDWKDHKFKWRKDPQDK

SEQ ID NO: 5 - protein, 21 aa, synthetic construct
VSFCLVMVLLFAVDTGLYFSV

SEQ ID NO: 6 - protein, 46 aa, synthetic construct
VSFCLVMVLLFAVDTGLYFSVKTNIRSSTRDWKDHKFKWRKDPQDK

SEQ ID NO: 7 - protein, 44 aa, synthetic construct
GSTVYYQGKCLTWKGPRRQLPAVVPAPLPPPCGSSAHLLPPVPG

SEQ ID NO: 8 - protein, 21 aa, synthetic construct
AGTVLLLRAGFYAVSFLSVAV

SEQ ID NO: 9 - protein, 65 aa, synthetic construct
AGTVLLLRAGFYAVSFLSVAVGSTVYYQGKCLTWKGPRRQLPAVVPAPLPPPCGSSAHLLPPVPG

SEQ ID NO: 10 - protein, 138 aa, synthetic construct
IPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 11 - protein, 23 aa, synthetic construct
LTAEVLGIICIVLMATVLKTIVL

SEQ ID NO: 12 - protein, 161 aa, synthetic construct
LTAEVLGIICIVLMATVLKTIVLIPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 13 - protein, 119 aa, synthetic construct
WRRKRKEKQSETSPKEFLTIYEDVKDLKTRRNHEQEQTFPGGGSTIYSMIQSQSSAPTSQEPAYTLYSLIPSRKSGSRKRNHSPSFNSTIYEVIGKSQPKAQNPARLSRKELENFDVYS

SEQ ID NO: 14 - protein, 21 aa, synthetic construct
FLVIIVILSALFLGTLACFCV

SEQ ID NO: 15 - protein, 140 aa, synthetic construct
FLVIIVILSALFLGTLACFCVWRRKRKEKQSETSPKEFLTIYEDVKDLKTRRNHEQEQTFPGGGSTIYSMIQSQSSAPTSQEPAYTLYSLIPSRKSGSRKRNHSPSFNSTIYEVIGKSQPKAQNPARLSRKELENFDVYS

SEQ ID NO: 16 - protein, 61 aa, synthetic construct
NRRRRRERRDLFTESWDTQKAPNNYRSPISTSQPTNQSMDDTREDIYVNYPTFSRRPKTRV

SEQ ID NO: 17 - protein, 21 aa, synthetic construct
GGTVLLLLFVISITTIIVIFL

SEQ ID NO: 18 - protein, 82 aa, synthetic construct
GGTVLLLLFVISITTIIVIFLNRRRRRERRDLFTESWDTQKAPNNYRSPISTSQPTNQSMDDTREDIYVNYPTFSRRPKTRV

SEQ ID NO: 19 - protein, 42 aa, synthetic construct
KRGRKKLLYIFKQPFMRPVQTTQEEDGCSCRFPEEEEGGCEL

SEQ ID NO: 20 - protein, 27 aa, synthetic construct
IISFFLALTSTALLFLLFFLTLRFSVV

SEQ ID NO: 21 - protein, 69 aa, synthetic construct
IISFFLALTSTALLFLLFFLTLRFSVVKRGRKKLLYIFKQPFMRPVQTTQEEDGCSCRFPEEEEGGCEL

SEQ ID NO: 22 - protein, 132 aa, synthetic construct
VSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 23 - protein, 27 aa, synthetic construct
LLLVASVIQGLGLLLCFTYICLHFSAL

SEQ ID NO: 24 - protein, 159 aa, synthetic construct
LLLVASVIQGLGLLLCFTYICLHFSALVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 25 - protein, 48 aa, synthetic construct
QRRKYRSNKGESPVEPAEPCHYSCPREEEGSTIPIQEDYRKPEPACSP

SEQ ID NO: 26 - protein, 21 aa, synthetic construct
ILVIFSGMFLVFTLAGALFLH

SEQ ID NO: 27 - protein, 69 aa, unidentified
ILVIFSGMFLVFTLAGALFLHQRRKYRSNKGESPVEPAEPCHYSCPREEEGSTIPIQEDYRKPEPACSP

SEQ ID NO: 28 - protein, 41 aa, synthetic construct
LLHRWCSNKKNASVMDQGPAGNRTVNREDSDEQDHQEVSYA

SEQ ID NO: 29 - protein, 19 aa, synthetic construct
VLIGTSVVKLPFTILLFFL

SEQ ID NO: 30 - protein, 60 aa, synthetic construct
VLIGTSVVKLPFTILLFFLLLHRWCSNKKNASVMDQGPAGNRTVNREDSDEQDHQEVSYA

SEQ ID NO: 31 - protein, 22 aa, synthetic construct
HRWCSNKKKCCCNGPRACREQK

SEQ ID NO: 32 - protein, 20 aa, synthetic construct
ILIGTSVVKIPFTILLFFLL

SEQ ID NO: 33 - protein, 42 aa, synthetic construct
ILIGTSVVKIPFTILLFFLLHRWCSNKKKCCCNGPRACREQK

SEQ ID NO: 34 - protein, 38 aa, synthetic construct
MQDEERYMTLNVQSKKRSSAQTSQLTFKDYSVTLHWYK

SEQ ID NO: 35 - protein, 21 aa, synthetic construct
ILLGISGTVNGILTLTLISLI

SEQ ID NO: 36 - protein, 59 aa, synthetic construct
MQDEERYMTLNVQSKKRSSAQTSQLTFKDYSVTLHWYKILLGISGTVNGILTLTLISLI

SEQ ID NO: 37 - protein, 51 aa, synthetic construct; identical to SEQ ID NO: 1

SEQ ID NO: 38 - protein, 216 aa, synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 39 - protein, 208 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 40 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 41 - protein, 135 aa, synthetic construct
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLG

SEQ ID NO: 42 - protein, 201 aa, synthetic construct
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 43 - protein, 70 aa, synthetic construct
MSKQRGTFSEVSLAQDPKRQQRKPKGNKSSISGTEQEIFQVELNLQNPSLNHQGIDKIYDCQGLLPPPEK

SEQ ID NO: 44 - protein, 231 aa, synthetic construct
MSKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 45 - protein, 229 aa, synthetic construct
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWP

SEQ ID NO: 46 - protein, 370 aa, synthetic construct
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 47 - protein, 99 aa, synthetic construct
MTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVA

SEQ ID NO: 48 - protein, 181 aa, synthetic construct
MTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 49 - protein, 186 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 50 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 51 - protein, 23 aa, synthetic construct
MERVQPLEENVGNAARPRFERNK

SEQ ID NO: 52 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 53 - protein, 145 aa, synthetic construct
MNAVIISPSSLQFQKLRPVYTRIAGFKVAPLNKCSLARHFLEPGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 54 - protein, 214 aa, synthetic construct
MNAVIISPSSLQFQKLRPVYTRIAGFKVAPLNKCSLARHFLEPGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 55 - protein, 245 aa, synthetic construct
MSLMVISMACVAFFLLQGAWPHEGFRRKPSLLAHPGPLVKSEETVILQCWSDVMFEHFLLHREGTFNHTLRLIGEHIDGVSKGNFSIGRMTQDLAGTYRCYGSVTHSPYQLSAPSDPLDIVITGLYEKPSLSAQPGPTVLAGESVTLSCSSRSSYDMYHLSREGEAHERRLPAGPKVNRTFQADFPLDPATHGGTYRCFGSFRDSPYEWSKSSDPLLVSVTGNSSNSWPSPTEPSSETGNPRHLH

SEQ ID NO: 56 - protein, 304 aa, synthetic construct
MSLMVISMACVAFFLLQGAWPHEGFRRKPSLLAHPGPLVKSEETVILQCWSDVMFEHFLLHREGTFNHTLRLIGEHIDGVSKGNFSIGRMTQDLAGTYRCYGSVTHSPYQLSAPSDPLDIVITGLYEKPSLSAQPGPTVLAGESVTLSCSSRSSYDMYHLSREGEAHERRLPAGPKVNRTFQADFPLDPATHGGTYRCFGSFRDSPYEWSKSSDPLLVSVTGNSSNSWPSPTEPSSETGNPRHLHVLIGTSVVKLPFTILLFFLLHRWCSNKKNASVMDQGPAGNRTVNREDSDEQDHQEVSYA

SEQ ID NO: 57 - protein, 340 aa, synthetic construct
MLLMVVSMACVGLFLVQRAGPHMGGQDKPFLSAWPSAVVPRGGHVTLRCHYRHRFNNFMLYKEDRIHVPIFHGRIFQEGFNMSPVTTAHAGNYTCRGSHPHSPTGWSAPSNPMVIMVTGNHRKPSLLAHPGPLVKSGERVILQCWSDIMFEHFFLHREWISKDPSRLVGQIHDGVSKANFSIGSMMRALAGTYRCYGSVTHTPYQLSAPSDPLDIVVTGLYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNLRHLH

SEQ ID NO: 58 - protein, 382 aa, synthetic construct
MLLMVVSMACVGLFLVQRAGPHMGGQDKPFLSAWPSAVVPRGGHVTLRCHYRHRFNNFMLYKEDRIHVPIFHGRIFQEGFNMSPVTTAHAGNYTCRGSHPHSPTGWSAPSNPMVIMVTGNHRKPSLLAHPGPLVKSGERVILQCWSDIMFEHFFLHREWISKDPSRLVGQIHDGVSKANFSIGSMMRALAGTYRCYGSVTHTPYQLSAPSDPLDIVVTGLYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNLRHLHILIGTSVVKIPFTILLFFLLHRWCSNKKKCCCNGPRACREQK

SEQ ID NO: 59 - protein, 172 aa, synthetic construct
LLVSQGVLLKCQKGSCSNATQYEDTGDLKVNNGTRRNISNKDLCASRSADQTVLCQSEWLKYQGKCYWFSNEMKSWSDSYVYCLERKSHLLIIHDQLEMAFIQKNLRQLNYVWIGLNFTSLKMTWTWVDGSPIDSKIFFIKGPAKENSCAAIKESKIFSETCSSVFKWICQY

SEQ ID NO: 60 - protein, 231 aa, synthetic construct
MQDEERYMTLNVQSKKRSSAQTSQLTFKDYSVTLHWYKILLGISGTVNGILTLTLISLILLVSQGVLLKCQKGSCSNATQYEDTGDLKVNNGTRRNISNKDLCASRSADQTVLCQSEWLKYQGKCYWFSNEMKSWSDSYVYCLERKSHLLIIHDQLEMAFIQKNLRQLNYVWIGLNFTSLKMTWTWVDGSPIDSKIFFIKGPAKENSCAAIKESKIFSETCSSVFKWICQY

SEQ ID NO: 61 - protein, 8 aa, synthetic construct
GFTFSNYG

SEQ ID NO: 62 - protein, 8 aa, synthetic construct
GYTFTSYW

SEQ ID NO: 63 - protein, 8 aa, synthetic construct
GFSLSRYS

SEQ ID NO: 64 - protein, 8 aa, unidentified
ISSGGGDT

SEQ ID NO: 65 - protein, 8 aa, synthetic construct
IHPSDSET

SEQ ID NO: 66 - protein, 7 aa, synthetic construct
IWGGGYT

SEQ ID NO: 67 - protein, 13 aa, synthetic construct
ARDYGGTWDYFDY

SEQ ID NO: 68 - protein, 15 aa, synthetic construct
AREEGQLGHGGAMDY

SEQ ID NO: 69 - protein, 11 aa, synthetic construct
ARYIDSSGYDY

SEQ ID NO: 70 - protein, 6 aa, synthetic construct
QDISKY

SEQ ID NO: 71 - protein, 6 aa, synthetic construct
QTVNDD

SEQ ID NO: 72 - protein, 5 aa, synthetic construct
SSVSY

SEQ ID NO: 73 - protein, 6 aa, synthetic construct
ENIYSY

SEQ ID NO: 74 - protein, 11 aa, synthetic construct
AAWDDSVRGPV

SEQ ID NO: 75 - protein, 115 aa, synthetic construct
MAQVQLQQSGAEVKKPGESLKISCKGSGYIFTGYYMHWVRQAPGQRPEWLGRMNAKSGEADSAQRFQGRVTMTRDTSINTAYMELRDLRSDDTAVYYCTRGPLLWGQGTLVTVSS

SEQ ID NO: 76 - protein, 110 aa, synthetic construct
QPVLTQPPSASGTPGQRVTISCSGSSSNIGSNFVSWYQQLPGTAPKLLIYRNNQRPSGVPDRFSGSKTGTSASLAISGLRSEDEADYYCAAWDDSVRGPVFGGGTELTVL

SEQ ID NO: 77 - protein, 8 aa, synthetic construct
SSNIGSNF

SEQ ID NO: 78 - protein, 9 aa, synthetic construct
QQGDTFPWT

SEQ ID NO: 79 - protein, 9 aa, synthetic construct
QQDYSSPYT

SEQ ID NO: 80 - protein, 9 aa, synthetic construct
QQWSSNPLT

SEQ ID NO: 81 - protein, 9 aa, synthetic construct
QHHYGTPYT

SEQ ID NO: 82 - protein, 120 aa, synthetic construct
EVKLVESGGGLVKPGASLKLSCAASGFTFSNYGMSWVRQTSDKRLEWVASISSGGGDTYYPDNVKGRFTISRENAKNTLYLQMSSLNSEDTALYYCARDYGGTWDYFDYWGQGTTLTVSS

SEQ ID NO: 83 - protein, 122 aa, synthetic construct
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSS

SEQ ID NO: 84 - protein, 117 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSS

SEQ ID NO: 85 - protein, 107 aa, synthetic construct
DIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 86 - protein, 107 aa, synthetic construct
SIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 87 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 88 - protein, 107 aa, synthetic construct
DIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 89 - protein, 15 aa, synthetic construct
GGGGSGGGGSGGGGS

SEQ ID NO: 90 - protein, 242 aa, synthetic construct
EVKLVESGGGLVKPGASLKLSCAASGFTFSNYGMSWVRQTSDKRLEWVASISSGGGDTYYPDNVKGRFTISRENAKNTLYLQMSSLNSEDTALYYCARDYGGTWDYFDYWGQGTTLTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 91 - protein, 244 aa, synthetic construct
QVQLQQPGAELVRPGVSVKLSCKASGYTFTSYWMNWVKQRPGQGLEWIGMIHPSDSETRLNQKFKDKAILTVDKSSSTAYMQLSSPTSEDSAVYYCAREEGQLGHGGAMDYWGQGTSVTVSSGGGGSGGGGSGGGGSDIQMTQTTSSLSASLGDRVTISCRASQDISKYLNWYQQKPDGTVKLLIYYTSRLHSGVPSRFSGSGSGTDYSLTISNLEQEDIATYFCQQGDTFPWTFGGGTKLEIK

SEQ ID NO: 92 - protein, 239 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSSIVMTQTPKFLLVSAGDRVTITCKASQTVNDDVAWYQQKPGQSPKLLIYYVSNRHTGVPDRFTGSGYGTDFTFTISTVQAEDLAVYFCQQDYSSPYTFGGGTKLEIK

SEQ ID NO: 93 - protein, 238 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSQIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 94 - protein, 239 aa, synthetic construct
QVQLKESGPGLVAPSQSLSITCTVSGFSLSRYSVHWVRQPPGKGLEWLGMIWGGGYTDYNSALKSRLSISKDNSKSQVFLKMNSLQTDDTAMYYCARYIDSSGYDYWGQGTTLTVSSGGGGSGGGGSGGGGSDIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGGGTKLEIK

SEQ ID NO: 95 - protein, 8 aa, synthetic construct
GYTFTDYN

SEQ ID NO: 96 - protein, 8 aa, synthetic construct
INPNNGGT

SEQ ID NO: 97 - protein, 14 aa, synthetic construct
ARKGYGGNYDYFDY

SEQ ID NO: 98 - protein, 6 aa, synthetic construct
QSIGTS

SEQ ID NO: 99 - skipped

SEQ ID NO: 100 - protein, 9 aa, synthetic construct
QQSNSWPYT

SEQ ID NO: 101 - protein, 8 aa, synthetic construct
GFNIKDTY

SEQ ID NO: 102 - protein, 10 aa, synthetic construct
GFSLSTYGMG

SEQ ID NO: 103 - protein, 8 aa, synthetic construct
IDPANGNT

SEQ ID NO: 104 - protein, 7 aa, synthetic construct
IYWDDDK

SEQ ID NO: 105 - protein, 11 aa, synthetic construct
ALYYYGGSLDY

SEQ ID NO: 106 - protein, 14 aa, synthetic construct
AQSLIYDGYYGFAY

SEQ ID NO: 107 - protein, 12 aa, synthetic construct
QSLLYSGNQKNY

SEQ ID NO: 108 - skipped

SEQ ID NO: 109 - protein, 9 aa, synthetic construct
QQYYSYPRT

SEQ ID NO: 110 - protein, 8 aa, unidentified
GYTFTYYG

SEQ ID NO: 111 - protein, 8 aa, synthetic construct
INTYSGVP

SEQ ID NO: 112 - protein, 14 aa, synthetic construct
ARWIYYSDLYGMDY

SEQ ID NO: 113 - protein, 11 aa, synthetic construct
QSIVHSNGDTY

SEQ ID NO: 114 - skipped

SEQ ID NO: 115 - protein, 9 aa, synthetic construct
FQGSHVPWT

SEQ ID NO: 116 - protein, 8 aa, synthetic construct
GYTFSSYW

SEQ ID NO: 117 - protein, 8 aa, synthetic construct
GYTLTTYL

SEQ ID NO: 118 - protein, 8 aa, synthetic construct
INPSSGYT

SEQ ID NO: 119 - protein, 8 aa, synthetic construct
INPNSGSS

SEQ ID NO: 120 - protein, 13 aa, synthetic construct
ARDGNYDHWYFDV

SEQ ID NO: 121 - protein, 12 aa, synthetic construct
AIRHYGGSLFDY

SEQ ID NO: 122 - protein, 6 aa, synthetic construct
QDINSY

SEQ ID NO: 123 - protein, 12 aa, synthetic construct
QSLLNSRTRKNY

SEQ ID NO: 124 - protein, 111 aa, synthetic construct
QPVLTQSSSASASLGSSVKLTCTLSSGHSGYIIAWHQQQPGKAPRYLMKVEGSGSYNKGSGIPERFSGSSSGADRYLTISNLQSEDEADYYCETWDSNTHVFGTGTKVTVL

SEQ ID NO: 125 - protein, 8 aa, synthetic construct
LQYDELLT

SEQ ID NO: 126 - protein, 11 aa, synthetic construct
AAWDDSVRGPV

SEQ ID NO: 127 - protein, 8 aa, synthetic construct
EQSYNLFT

SEQ ID NO: 128 - protein, 8 aa, synthetic construct
GYTFTDYN

SEQ ID NO: 129 - protein, 8 aa, synthetic construct
GFNIKDTY

SEQ ID NO: 130 - protein, 10 aa, synthetic construct
GFSLSTYGMG

SEQ ID NO: 131 - protein, 8 aa, synthetic construct
GYTFTYYG

SEQ ID NO: 132 - protein, 8 aa, synthetic construct
GYTFSSYW

SEQ ID NO: 133 - protein, 8 aa, synthetic construct
GYTLTTYL

SEQ ID NO: 134 - protein, 8 aa, synthetic construct
INPSSGYT

SEQ ID NO: 135 - protein, 8 aa, synthetic construct
INPNSGSS

SEQ ID NO: 136 - protein, 13 aa, synthetic construct
ARDGNYDHWYFDV

SEQ ID NO: 137 - protein, 12 aa, synthetic construct
AIRHYGGSLFDY

SEQ ID NO: 138 - protein, 12 aa, synthetic construct
QSLLNSRTRKNY

SEQ ID NO: 139 - protein, 8 aa, synthetic construct
GYTFTSNY

SEQ ID NO: 140 - protein, 8 aa, synthetic construct
LQYDELLT

SEQ ID NO: 141 - protein, 8 aa, synthetic construct
EQSYNLFT

SEQ ID NO: 142 - protein, 121 aa, synthetic construct
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSS

SEQ ID NO: 143 - protein, 118 aa, synthetic construct
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSS

SEQ ID NO: 144 - protein, 122 aa, synthetic construct
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSA

SEQ ID NO: 145 - protein, 121 aa, synthetic construct
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSS

SEQ ID NO: 146 - protein, 120 aa, synthetic construct
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSS

SEQ ID NO: 147 - protein, 119 aa, synthetic construct
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSS

SEQ ID NO: 148 - protein, 107 aa, synthetic construct
DILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 149 - protein, 113 aa, synthetic construct
DIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 150 - protein, 112 aa, synthetic construct
DVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 151 - protein, 106 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 152 - protein, 112 aa, synthetic construct
DIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 153 - protein, 243 aa, synthetic construct
EVQLQQSGPELVKPGSSVKISCKASGYTFTDYNMDWVKQSHGKSLEWIGTINPNNGGTSYNQKFKGKATLTVDKSSSTAYMELRSLTSEDSAVYYCARKGYGGNYDYFDYWGQGTTLTVSSGGGGSGGGGSGGGGSDILLTQSPAILSVSPGERVSFSCRASQSIGTSIHWYQQRTNGSPRLLIKYASESISGIPSRFSGSGSGTDFTLSINSVESEDIADYYCQQSNSWPYTFGGGTKLEIK

SEQ ID NO: 154 - protein, 246 aa, synthetic construct
EVQLQQSGAELVKPGASVKLSCTASGFNIKDTYMHWVKQRPEQGLEWIGRIDPANGNTIYASKFQGKATITADTSSNTAYMQLSSLTSGDTAVYYCALYYYGGSLDYWGQGTTLTVSSGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 155 - protein, 250 aa, synthetic construct
QVTLKESGPGILQPSQTLSLTCSFSGFSLSTYGMGVSWIRQPSGKGLEWLAHIYWDDDKRYNPSLKSRLTISKDTSNNQVFLKITSVDTADTATYYCAQSLIYDGYYGFAYWGQGTLVTVSAGGGGSGGGGSGGGGSDIVMSQSPSSLAVSVGERVTMSCKSSQSLLYSGNQKNYLAWYQQKPGQSPKLLIYWASTRESGVPDRFTGSGSGTDFTLTISSVKAEDLAVYYCQQYYSYPRTFGGGTKLEIK

SEQ ID NO: 156 - protein, 248 aa, synthetic construct
QIQLVQSGPELKKPGETVKISCKASGYTFTYYGMNWVKQAPGKGLEWMGWINTYSGVPTYADDFKGRFAFSLETSVSTAYLQINNLKNEDTATYFCARWIYYSDLYGMDYWGQGTSVTVSSGGGGSGGGGSGGGGSDVLMTQSPLSLPVSLGDQASISCRSSQSIVHSNGDTYLEWYLQKPGQSPKLLIYKVSNRFSGVPDRFSGSGSGTDFTLKISRVEAEDLGVYHCFQGSHVPWTFGGGTKLEIK

SEQ ID NO: 157 - protein, 241 aa, synthetic construct
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 158 - protein, 247 aa, synthetic construct
QVQLQQSGAELAKPGASVKMSCKASGYTFSSYWMHWLKQRPGQGLEWIGYINPSSGYTNYNQKFKDKATLTADKSSSTAYMQLSSLTSEDSAVYYCARDGNYDHWYFDVWGTGTTVTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 159 - protein, 240 aa, synthetic construct
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDELLTFGAGTKLELK

SEQ ID NO: 160 - protein, 246 aa, synthetic construct
QVQLQQPGAELVRPGASVKMSCKASGYTLTTYLMDWVKQRLGQGFEWIGNINPNSGSSNYNEKFKGKAKLTVDKSSSTAYMQLSSLTSEDSAVYYCAIRHYGGSLFDYWGQGTTLTVSSGGGGSGGGGSGGGGSDIVMSQSPSSLAVSAGERVTMSCRSSQSLLNSRTRKNYLAWYQQKPGQSPKLLIYWASTRESGVPDRFSGSGSGTDFTLTISSVQAEDLAVYYCEQSYNLFTFGSGTKLEIK

SEQ ID NO: 161 - protein, 8 aa, synthetic construct
GFDIKDTY

SEQ ID NO: 162 - protein, 5 aa, synthetic construct
TYAMY

SEQ ID NO: 163 - protein, 4 aa, synthetic construct
TFWM

SEQ ID NO: 164 - protein, 5 aa, synthetic construct
TFWMQ

SEQ ID NO: 165 - protein, 8 aa, synthetic construct
IDPANGDT

SEQ ID NO: 166 - protein, 22 aa, synthetic construct
RIRSKVNNYATYYADSVKDRFT

SEQ ID NO: 167 - protein, 20 aa, synthetic construct
TIYPGDDDTRYTQKFKGRAT

SEQ ID NO: 168 - protein, 7 aa, synthetic construct
ARRGGLS

SEQ ID NO: 169 - protein, 5 aa, synthetic construct
DPMDY

SEQ ID NO: 170 - protein, 12 aa, synthetic construct
SGYERGPYYFDS

SEQ ID NO: 171 - protein, 10 aa, synthetic construct
SGYERGPYYF

SEQ ID NO: 172 - protein, 6 aa, synthetic construct
GNIHNY

SEQ ID NO: 173 - protein, 16 aa, synthetic construct
GSSKSLLHSNGNTYLY

SEQ ID NO: 174 - protein, 17 aa, synthetic construct
KSSQSLLCRSNQKNYLA

SEQ ID NO: 175 - protein, 17 aa, synthetic construct
KSSQSLLYRSNQKNYLA

SEQ ID NO: 176 - protein, 8 aa, synthetic construct
INPSGGRT

SEQ ID NO: 177 - protein, 7 aa, synthetic construct
RVSNLAS

SEQ ID NO: 178 - protein, 7 aa, synthetic construct
WASTRES

SEQ ID NO: 179 - protein, 9 aa, synthetic construct
QHFWSTPWT

SEQ ID NO: 180 - protein, 9 aa, synthetic construct
MQHLEYPYT

SEQ ID NO: 181 - protein, 9 aa, synthetic construct
QQYYSYPLT

SEQ ID NO: 182 - protein, 114 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 183 - protein, 116 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 184 - protein, 116 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 185 - protein, 121 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 186 - protein, 121 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 187 - protein, 121 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 188 - protein, 107 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 189 - protein, 112 aa, synthetic construct
GNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 190 - protein, 112 aa, synthetic construct
GNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 191 - protein, 112 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 192 - protein, 112 aa, synthetic construct; identical to SEQ ID NO: 191

SEQ ID NO: 193 - protein, 113 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 194 - protein, 113 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 195 - protein, 236 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 196 - protein, 241 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 197 - protein, 241 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 198 - protein, 241 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 199 - protein, 242 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 200 - protein, 242 aa, synthetic construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 201 - protein, 238 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 202 - protein, 243 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 203 - protein, 243 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 204 - protein, 243 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 205 - protein, 244 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 206 - protein, 244 aa, synthetic construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 207 - protein, 238 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 208 - protein, 243 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 209 - protein, 243 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 210 - protein, 243 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 211 - protein, 244 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 212 - protein, 244 aa, synthetic construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 213 - protein, 243 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 214 - protein, 248 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 215 - protein, 248 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 216 - protein, 248 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 217 - protein, 249 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 218 - protein, 249 aa, synthetic construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 219 - protein, 243 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 220 - protein, 248 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 221 - protein, 248 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 222 - protein, 248 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 223 - protein, 248 aa, synthetic construct; identical to SEQ ID NO: 222

SEQ ID NO: 224 - protein, 249 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 225 - protein, 249 aa, synthetic construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 226 - protein, 243 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 227 - protein, 248 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 228 - protein, 248 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 229 - protein, 248 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 230 - protein, 248 aa, synthetic construct; identical to SEQ ID NO: 229

SEQ ID NO: 231 - protein, 249 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 232 - protein, 249 aa, synthetic construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 233 - protein, 236 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 234 - protein, 238 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 235 - protein, 238 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 236 - protein, 243 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 237 - protein, 243 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 238 - protein, 243 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 239 - protein, 241 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 240 - protein, 243 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 241 - protein, 243 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 242 - protein, 248 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 243 - protein, 248 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 244 - protein, 248 aa, synthetic construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 245 - protein, 242 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 246 - protein, 244 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 247 - protein, 244 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 248 - protein, 249 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 249 - protein, 249 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 250 - protein, 249 aa, synthetic construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 251 - protein, 242 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 252 - protein, 244 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 253 - protein, 244 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 254 - protein, 249 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 255 - protein, 249 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 256 - protein, 249 aa, synthetic construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 257 - protein, 8 aa, synthetic construct
GFTFSSFA

SEQ ID NO: 258 - protein, 5 aa, synthetic construct
SFAVS

SEQ ID NO: 259 - protein, 5 aa, synthetic construct
SHDMS

SEQ ID NO: 260 - protein, 8 aa, synthetic construct
ISSGGAYT

SEQ ID NO: 261 - protein, 20 aa, synthetic construct
TISSGGAYTFYKDSVKGRFT

SEQ ID NO: 262 - protein, 20 aa, synthetic construct
YISGGGTNIYYSDTVKGRFT

SEQ ID NO: 263 - protein, 16 aa, synthetic construct
ARHSGYDGYYLYAMDY

SEQ ID NO: 264 - protein, 14 aa, synthetic construct
HSGYDGYYLYAMDY

SEQ ID NO: 265 - protein, 12 aa, synthetic construct
PNYNYGGSWFAY

SEQ ID NO: 266 - protein, 5 aa, synthetic construct
SSVHY

SEQ ID NO: 267 - protein, 9 aa, synthetic construct
ASSSVHYMH

SEQ ID NO: 268 - protein, 10 aa, synthetic construct
SASSSVHYMH

SEQ ID NO: 269 - protein, 15 aa, synthetic construct
ARSHCSGGSCYSIDY

SEQ ID NO: 270 - protein, 7 aa, synthetic construct
DTSKLAS

SEQ ID NO: 271 - protein, 9 aa, synthetic construct
QQWTSNPPT

SEQ ID NO: 272 - protein, 123 aa, synthetic construct
ELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 273 - DNA, 369 bp, synthetic construct
  source 1..369: mol_type = other DNA
gaactaatactggtggagtctgggggaggcttagtgaagcctggagggtccctgaaactctcctgtgcagtctctggattcactttcagttcctttgccatgtcttgggttcgccagactccggagaagaggctggagtgggtcgcaaccattagtagtggtggagcttacaccttctataaagacagtgtgaaggggcgattcaccatctccagagacaatgccaagaataccctgtacctgcaaatgagcagtctgaggtctgaggactcggccatgtattactgtgcaagacatagcggctatgatggttactacctctatgctatggactactggggtcaaggaacctcagtcaccgtctcctca

SEQ ID NO: 274 - protein, 127 aa, synthetic construct
GVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 275 - DNA, 381 bp, synthetic construct
  source 1..381: mol_type = other DNA
ggtgtccagtgtgaactaatactggtggagtctgggggaggcttagtgaagcctggagggtccctgaaactctcctgtgcagtctctggattcactttcagttcctttgccgtgtcctgggttcgccagactccggagaagaggctggagtgggtcgcaaccattagtagtggtggagcttacaccttctataaagacagtgtgaaggggcgattcaccatctccagagacaatgccaagaataccctgtacctgcaaatgagcagtctgaggtctgaggactcggccatgtattactgtgcaagacatagcggctatgatggttactacctctatgctatggactactggggtcaaggaacctcagtcaccgtctcctca

SEQ ID NO: 276 - protein, 121 aa, synthetic construct
EVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 277 - DNA, 363 bp, synthetic construct
  source 1..363: mol_type = other DNA
gaggtgcagctggaggagtctgggggaggcttagtgcagccgggagggtccctgaaagtctcctgtgcagtttccggactcgctttcagcagccatgacatgtcttgggttcgccagactccggagaagcggctggagtgggtcgcatacattagtggaggtggtactaatatctattattcagacactgtgaagggccgattcaccatctccagagacaatgccaagaacaccctgtacctgcaaatgagcagtctgaagtctgaagacacagccatttattactgtgcaagacccaattataattacggcggttcctggtttgcttactggggccaagggactctggtcactgtctctgca

SEQ ID NO: 278 - protein, 106 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 279 - DNA, 320 bp, synthetic construct
  source 1..320: mol_type = other DNA
caaattgttctcacccagtctccagaaatcatgtctgcatctccaggggagaaggtcaccatgacctgcagtgccagctcaagtgtacattacatgcactggtaccagcagaagtcaggcacctcccccaaaagatggatttatgacacatccaaactggcttctggagtccctggtcgcttcagtggcagtgggtctgggacctcttactctctcacaatcagcagcatggagtctgaagatgctgccacttattactgccagcagtggactagtaacccacccacgttcggaggggggaccaagctggaaattaaacg

SEQ ID NO: 280 - protein, 244 aa, synthetic construct
ELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 281 - protein, 248 aa, synthetic construct
GVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 282 - protein, 242 aa, synthetic construct
EVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSAGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 283 - protein, 244 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 284 - protein, 248 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSGVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 285 - protein, 242 aa, synthetic construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSEVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 286 - protein, 12 aa, synthetic construct
GFSITTGGYWWT

SEQ ID NO: 287 - protein, 5 aa, synthetic construct
SDGIS

SEQ ID NO: 288 - protein, 5 aa, synthetic construct
SNAMI

SEQ ID NO: 289 - protein, 17 aa, synthetic construct
GYIFSSGNTNYNPSIKS

SEQ ID NO: 290 - protein, 16 aa, synthetic construct
IISSGGNTYYASWAKG

SEQ ID NO: 291 - protein, 16 aa, synthetic construct
AMDSNSRTYYATWAKG

SEQ ID NO: 292 - protein, 12 aa, synthetic construct
CARAYGKLGFDY

SEQ ID NO: 293 - protein, 8 aa, synthetic construct
VVGGTYSI

SEQ ID NO: 294 - protein, 11 aa, synthetic construct
GDGGSSDYTEM

SEQ ID NO: 295 - protein, 12 aa, synthetic construct
TLSSQHSTYTIG

SEQ ID NO: 296 - protein, 13 aa, synthetic construct
QSSQSVYNNDFLS

SEQ ID NO: 297 - protein, 13 aa, synthetic construct
QSSQSVYGNNELS

SEQ ID NO: 298 - protein, 11 aa, synthetic construct
VNSDGSHSKGD

SEQ ID NO: 299 - protein, 7 aa, synthetic construct
YASTLAS

SEQ ID NO: 300 - protein, 7 aa, synthetic construct
QASSLAS

SEQ ID NO: 301 - protein, 9 aa, synthetic construct
GSSDSSGYV

SEQ ID NO: 302 - protein, 13 aa, synthetic construct
TGTYGNSAWYEDA

SEQ ID NO: 303 - protein, 11 aa, synthetic construct
LGEYSISADNH

SEQ ID NO: 304 - protein, 12 aa, synthetic construct
GFSITTGGYWWT

SEQ ID NO: 305 - protein, 17 aa, synthetic construct
GYIFSSGNTNYNPSIKS

SEQ ID NO: 306 - protein, 12 aa, synthetic construct
CARAYGKLGFDY

SEQ ID NO: 307 - protein, 12 aa, synthetic construct
TLSSQHSTYTIG

SEQ ID NO: 308 - protein, 11 aa, synthetic construct
VNSDGSHSKGD

SEQ ID NO: 309 - protein, 9 aa, synthetic construct
GSSDSSGYV

SEQ ID NO: 310 - protein, 5 aa, synthetic construct
SDGIS

SEQ ID NO: 311 - protein, 16 aa, synthetic construct
IISSGGNTYYASWAKG

SEQ ID NO: 312 - protein, 8 aa, synthetic construct
VVGGTYSI

SEQ ID NO: 313 - protein, 13 aa, synthetic construct
QSSQSVYNNDFLS

SEQ ID NO: 314 - protein, 7 aa, synthetic construct
YASTLAS

SEQ ID NO: 315 - protein, 13 aa, synthetic construct
TGTYGNSAWYEDA

SEQ ID NO: 316 - protein, 5 aa, synthetic construct
SNAMI

SEQ ID NO: 317 - protein, 16 aa, synthetic construct
AMDSNSRTYYATWAKG

SEQ ID NO: 318 - protein, 11 aa, synthetic construct
GDGGSSDYTEM

SEQ ID NO: 319 - protein, 13 aa, synthetic construct
QSSQSVYGNNELS

SEQ ID NO: 320 - protein, 7 aa, synthetic construct
QASSLAS

SEQ ID NO: 321 - protein, 11 aa, synthetic construct
LGEYSISADNH

SEQ ID NO: 322 - protein, 120 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSS

SEQ ID NO: 323 - protein, 111 aa, synthetic construct
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 324 - protein, 454 aa, synthetic construct
METGLRWLLLVAVLKGVQCQSVEESGGRLVTPGTPLTLTCTVSGFSLSNNAINWVRQAPGKGLEWIGYIWSGGLTYYANWAEGRFTISKTSTTVDLKMTSPTIEDTATYFCARGINNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 325 - protein, 239 aa, synthetic construct
MDMRAPTQLLGLLLLWLPGARCADVVMTQTPASVSAAVGGTVTINCQASESISNYLSWYQQKPGQPPKLLIYRTSTLASGVSSRFKGSGSGTEYTLTISGVQCDDVATYYCQCTSGGKFISDGAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 326 - protein, 452 aa, synthetic construct
METGLRWLLLVAVLKGVQCQSVEESGGRLVTPGTPLTLTCTVSGFTISDYDLSWVRQAPGEGLKYIGFIAIDGNPYYATWAKGRFTISKTSTTVDLKITAPTTEDTATYFCARGAGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 327 - protein, 238 aa, synthetic construct
MDTREPTQLLGLLLLWLPGARCADVVMTQTPASVSAAVGGTVTINCQSSKNVYNNNWLSWFQQKPGQPPKLLIYYASTLASGVPSRFRGSGSGTQFTLTISDVQCDDAATYYCAGDYSSSSDNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 328 - protein, 454 aa, synthetic construct
METGLRWLLLVAVLKGVHCQSVEESGGRLVTPGTPLTLTCTASGFSRSSYDMSWVRQAPGKGLEWVGVISTAYNSHYASWAKGRFTISRTSTTVDLKMTSLTTEDTATYFCARGGSWLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 329 - protein, 239 aa, synthetic construct
MDXRAPTQLLGLLLLWLPGARCALVMTQTPASVSAAVGGTVTINCQSSQSVYDNDELSWYQQKPGQPPKLLIYALASKLASGVPSRFKGSGSGTQFALTISGVQCDDAATYYCQATHYSSDWYLTFGGGTEVVVKGFPVAPTVLLFPPSSDEVATGTVTIVCVANKYFPDVTVTWEVDGTTQTTGTENSKTPQNSADCTYNLSSTLTLTSTQYNSHKEYTCKVTQGTTSVVQSFSRKNC

SEQ ID NO: 330 - protein, 456 aa, synthetic construct
METGLRWLLLVAVLKGVQCQSVEESGGRLVTPGTPLTLTCTVSGFSLSSYDMTWVRQAPGKGLEWIGIIYASGTTYYANWAKGRFTISKTSTTVDLKVTSPTIGDTATYFCAREGAGVSMTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 331 - protein, 236 aa, synthetic construct
MDMRAPTQLLGLLLLWLPGARCAYDMTQTPASVEVAVGGTVTIKCQASQSISTYLDWYQQKPGQPPKLLIYDASDLASGVPSRFKGSGSGTQFTLTISDLECADAATYYCQQGYTHSNVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 332 - protein, 459 aa, synthetic construct
METGLRWLLLVAVLKGVQCQSVEESGGRLVSPGTPLTLTCTASGFSLSSYDMSWVRQAPGKGLEYIGIISSSGSTYYASWAKGRFTISKTSTTVDLEVTSLTTEDTATYFCSREHAGYSGDTGHLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVGIGPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 333 - protein, 236 aa, synthetic construct
MDMRAPTQLLGLLLLWLPGARCAYDMTQTPASVEVAVGGTVAIKCQASQSVSSYLAWYQQKPGQPPKPLIYEASMLAAGVSSRFKGSGSGTDFTLTISDLECDDAATYYCQQGYSISDIDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 334 - protein, 455 aa, synthetic construct
METGLRWLLLVAVLKGVQCQSVEESGGRLVTPGTPLTLTCTVSGIDLSSDGISWVRQAPGKGLEWIGIISSGGNTYYASWAKGRFTISRTSTTVDLKMTSLTTEDTATYFCARVVGGTYSIWGQGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 335 - protein, 240 aa, synthetic construct
MDTRAPTQLLGLLLLWLPGATFAQVLTQTASPVSAPVGGTVTINCQSSQSVYNNDFLSWYQQKPGQPPKLLIYYASTLASGVPSRFKGSGSGTQFTLTISDLECDDAATYYCTGTYGNSAWYEDAFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 336 - protein, 460 aa, synthetic construct
METGLRWLLLVAVLKGVQCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 337 - protein, 239 aa, synthetic construct
MDTRAPTQLLGLLLLWLPGATFAQAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 338 - protein, 221 aa, synthetic construct
QVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 339 - protein, 110 aa, synthetic construct
LTQPPPASGTPGQQRVTISCSGSSSNIGSNTVNWYQQLPGTAPKLLIYYGNDQRPSGVPDRFSASKSGTSASLAISGLQSEDEAHYYCAAWDGSLNGGVIFGGGTKVTLG

SEQ ID NO: 340 - protein, 109 aa, synthetic construct
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 341 - protein, 109 aa, synthetic construct
MTHTPLSLSVTPGQPASISCKSSQSLLHSDGKTYLYWYLQRPGQSPQPLIYEVSNRFSGVPDRFSGSGSGTDFTLKISRVQAEDVGVYYCMQSLQLWTFGQGTKVEIKR

SEQ ID NO: 342 - protein, 110 aa, synthetic construct
MTQSPLSLPVTLGQPASISCRSSQSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 343 - protein, 110 aa, synthetic construct
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 344 - protein, 110 aa, synthetic construct
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 345 - protein, 110 aa, synthetic construct
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 346 - protein, 92 aa, synthetic construct
MTQSPSSLSASVGDRVTITCQASQDISNYLNWYQQKPGKAPKLLIYDASNLETGVPSRFSGSGSGTDFTFTISSATYYCQQTYQGTKLEIKR

SEQ ID NO: 347 - protein, 105 aa, synthetic construct
MTQSPSSLSASVGHPVTITCRASQSLISYLNWYHQKPGKAPKLLIYAASILQSGVPSRFSGSGSGTDFTLTISSLQPENFASYYCQHTDSFPRTFGHGTKVEIKR

SEQ ID NO: 348 - protein, 108 aa, synthetic construct
LTQPPSASGTPGQGVTISCRGSTSNIGNNVVNWYQHVPGSAPKLLIWSNIQRPSGIPDRFSGSKSGTSASLAISGLQSEDQAVYYCAVWDDGLAGWVFGGGTTVTVLS

SEQ ID NO: 349 - protein, 105 aa, synthetic construct
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 350 - protein, 111 aa, synthetic construct
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGGQGTKLDIKR

SEQ ID NO: 351 - protein, 119 aa, synthetic construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 352 - protein, 119 aa, synthetic construct
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSS

SEQ ID NO: 353 - protein, 119 aa, synthetic construct
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 354 - protein, 119 aa, synthetic construct
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSS

SEQ ID NO: 355 - protein, 119 aa, synthetic construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSS

SEQ ID NO: 356 - protein, 120 aa, synthetic construct
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSS

SEQ ID NO: 357 - protein, 111 aa, synthetic construct
QLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 358 - protein, 111 aa, synthetic construct
LPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 359 - protein, 247 aa, synthetic construct
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAASSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAA

SEQ ID NO: 360 - protein, 253 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTV

SEQ ID NO: 361 - protein, 245 aa, synthetic construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 362 - protein, 245 aa, synthetic construct
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 363 - protein, 245 aa, synthetic construct
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 364 - protein, 245 aa, synthetic construct
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 365 - protein, 245 aa, synthetic construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 366 - protein, 246 aa, synthetic construct
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 367 - protein, 245 aa, synthetic construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 368 - protein, 245 aa, synthetic construct
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 369 - protein, 245 aa, synthetic construct
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 370 - protein, 245 aa, synthetic construct
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 371 - protein, 245 aa, synthetic construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 372 - protein, 246 aa, synthetic construct
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 373 - protein, 246 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 374 - protein, 274 aa, synthetic construct
MVLLVTSLLLCELPHPAFLLIPQVQLQESGPGLVKPSQTLSLTCTVSGGSISSGGYYWIWIRQHPGKGLEWIGYIYYNGNTYYNPSLKSRVTMSVDTSKNQFSLKLSSVTAADTAVYYCARDGITMIRGYYYGMDVWGQGTTVTVSSGGGGSGGGGSGGGGSDIQLTQSPSSVSASVGDRVTITCRASRGISSWLAWYQQKPGKAPKLLIYTASSLQSGVPSRFSGSGSGTDFTLTISSLQPEDFATYYCQQAYSFPRTFGQGTKVEIKAAAFV

SEQ ID NO: 375 - protein, 8 aa, synthetic construct
GYIFTGYY

SEQ ID NO: 376 - protein, 6 aa, synthetic construct
NAKSGE

SEQ ID NO: 377 - protein, 4 aa, synthetic construct
GPLL

SEQ ID NO: 378 - protein, 13 aa, synthetic construct
SSGSSNIGSNFVS

SEQ ID NO: 379 - protein, 7 aa, synthetic construct
RNNQRPS

SEQ ID NO: 380 - protein, 11 aa, synthetic construct
AAWDDSVRGPV

SEQ ID NO: 381 - protein, 112 aa, synthetic construct
QVQLQQSGAEVKKPGESLKISCKGSGYIFTGYYMHWVRQAPGQRPEWLGRMNAKSGEADSAQRFQGRVTMTRDTSINTAYMELRDLRSDDTAVYYCTRGPLLWGQGTLVTVS

SEQ ID NO: 382 - protein, 113 aa, synthetic construct
QPVLTQPPSASGTPGQRVTISCSGSSSNIGSNFVSWYQQLPGTAPKLLIYRNNQRPSGVPDRFSGSKTGTSASLAISGLRSEDEADYYCAAWDDSVRGPVFGGGTELTVLAAA

SEQ ID NO: 383 - protein, 8 aa, synthetic construct
GYIFTGYY

SEQ ID NO: 384 - protein, 8 aa, synthetic construct
MNAKSGEA

SEQ ID NO: 385 - protein, 6 aa, synthetic construct
TRGPLL

SEQ ID NO: 386 - protein, 8 aa, synthetic construct
SSNIGSNF

SEQ ID NO: 387 - protein, 122 aa, synthetic construct
EVQLVQSGAEVKKPGASVKVSCKASGYTFTSNYMHWVRQAPGQGLEWMGIINPSGGRTSYAQKFQGRVTMTRDTSTGTVYMELSSLRSEDTAVYYCARSHCSGGSCYSIDYWGQGTLVTVSS